(19) United States
(12) Patent Application Publication
(10) Pub. No.: US 2012/0206037 A1
(43) Pub. Date: Aug. 16, 2012

(54) NOVEL ORGANIC ELECTROLUMINESCENT COMPOUNDS AND ORGANIC ELECTROLUMINESCENT DEVICE USING THE SAME

(75) Inventors: Soo Young Lee, Namyangju-si (KR); Young Gil Kim, Anyang-si (KR); Youngg Jun Cho, Seongbuk-gu (KR); Hyuck Joo Kwon, Dongdaemun-gu (KR); Bong Ok Kim, Gangnam-gu (JP); Sung Min Kim, Yangcheon-gu (KR); Seung Soo Yoon, Suwon-si (KR)

(73) Assignee: Rohm and Haas Electronic Materials Korea Ltd, Chungeheongnam-do (KR)

(21) Appl. No.: 13/386,616

(22) PCT Filed: Jul. 19, 2010
(86) PCT No.: PCT/KR2010/004699

§ 371 Date: May 2, 2012

(30) Foreign Application Priority Data
Jul. 23, 2009 (KR) 1020090067370

(51) Int. Cl. H05B 33/14; C07D 403/04; C07F 7/10

(52) U.S. Cl. 313/504; 544/229; 544/212; 540/588

(57) ABSTRACT

Provided are a novel organic electroluminescent compound and an organic electroluminescent device using the same.

When used as a host material of an organic electroluminescent material of an OLED device, the organic electroluminescent compound disclosed herein exhibits good luminous efficiency and excellent life property as compared to the existing host material. Therefore, it may be used to manufacture OLEDs having very superior operation life.

### FIELD OF THE INVENTION

The present invention relates to novel organic electroluminescent compounds and an organic electroluminescent device using the same, more particularly, to a novel organic electroluminescent compound used as an electroluminescent material and an organic electroluminescent device using the same as host.

### BACKGROUND OF THE INVENTION

The most important factor that determines luminous efficiency of an OLED is the electroluminescent material. At present, fluorescent materials are widely used for the electroluminescent material. But, phosphorescent materials are better when considering the electroluminescence mechanism. Theoretically, phosphorescent materials can improve the luminous efficiency by 4-fold. Until now, iridium(III) complex-based phosphorescent materials are widely known. Such materials as (acac)Ir(btp)₂, Ir(ppy)₃ and Firpic are known for red, green and blue colors, respectively. Recently, a lot of researches on phosphorescent materials are underway, especially in Japan, Europe and US.

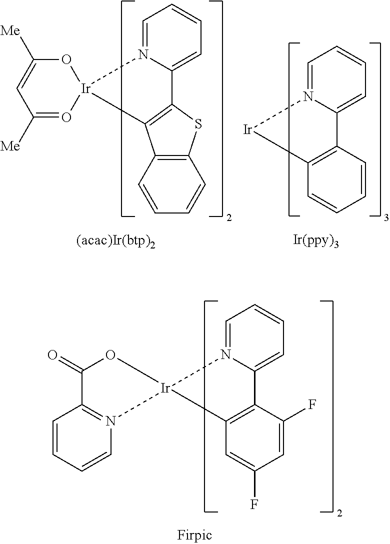

At present, CBP is the most widely known as a host material for a phosphorescent material. High-efficiency OLEDs using a hole blocking layer comprising BCP, BAlq, etc. are reported. High-performance OLEDs using BAlq derivatives as a host were reported by Pioneer (Japan) and others.

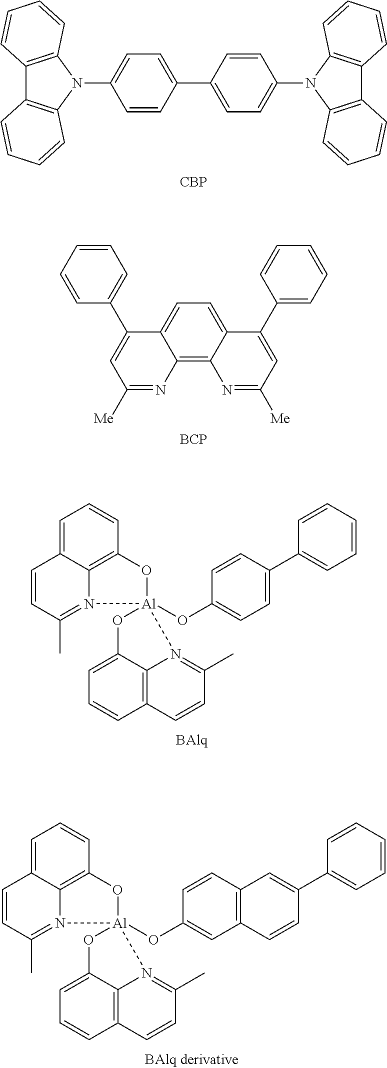

Although these materials provide good electroluminescence characteristics, they are disadvantageous in that degradation may occur during the high-temperature deposition process in vacuum because of low glass transition temperature and poor thermal stability. Since the power efficiency of an OLED is given by (π/voltage)×current efficiency, the power efficiency is inversely proportional to the voltage. High power efficiency is required to reduce the power consumption of an OLED. Actually, OLEDs using phosphorescent materials provide much better current efficiency (cd/A) than those using fluorescent materials. However, when the existing materials such as BAlq, CBP, etc. are used as a host of the phosphorescent material, there is no significant advantage in power efficiency (lm/W) over the OLEDs using fluorescent materials because of high driving voltage.

Further, the OLED devices do not have satisfactory operation life. Therefore, development of more stable, higher-performance host materials is required.

### DISCLOSURE

### Technical Problem

With intensive efforts to overcome the problems of conventional techniques as described above, the present inventors have invented novel electroluminescent compounds which can realize organic electroluminescent devices having excellent luminous efficiency and noticeably improved life property.

The object of the present invention is to provide organic electroluminescent compounds having the backbone to provide better luminous efficiency and device life with appropriate color coordinate as compared to conventional host or dopant material, while overcoming the problems described above.

### Technical Solution

Provided are a novel organic electroluminescent compound represented by Chemical Formulas 1 and 6 and an organic electroluminescent device using the same. Since the organic electroluminescent compound according to the present invention exhibits good luminous efficiency and excellent life property compared to the existing host material, it may be used to manufacture OLED devices having very superior operation life.

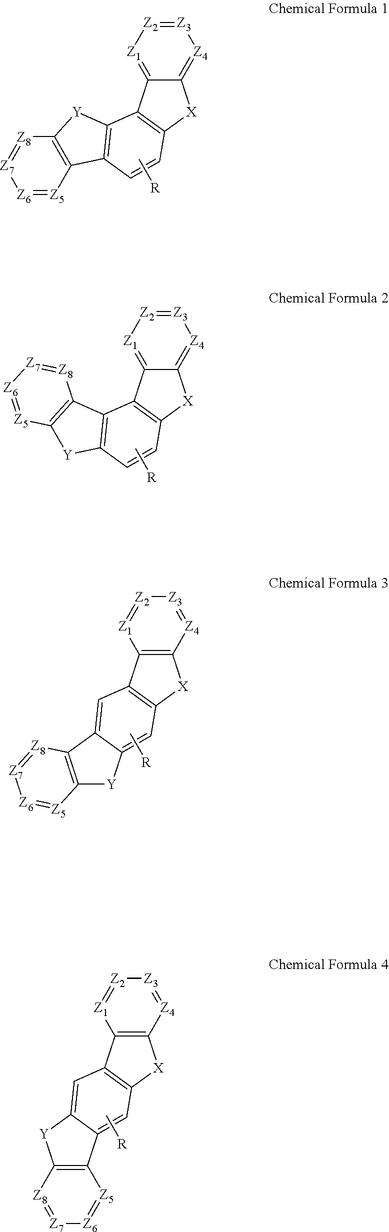

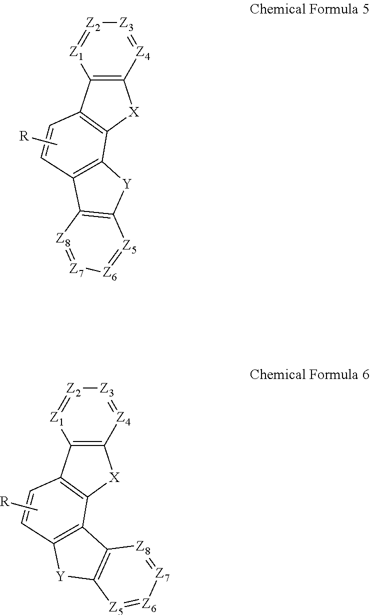

wherein,

X and Y independently represent N(R₁), C(R₂)(R₃) or Si (R₄)(R₅), with proviso that at least one of X and Y is (are) N(R₁) and the remaining is C(R₂)(R₃) or Si (R₄)(R₅);

Z₁ through Z₈ independently represent C(R₆) or N, wherein R₆ may be different from each other, and the neighboring R₆ may be linked to each other to form a ring;

R₁ through R₅ independently represent (C1-C30)alkyl, (C3-C30)cycloalkyl, 5- to 7-membered heterocycloalkyl, (C2-C30)alkenyl, (C2-C30)alkynyl, (C6-C30)aryl or (C3-C30)heteroaryl;

R and R₆ independently represent hydrogen, (C1-C30) alkyl, halogen, cyano, (C3-C30)cycloalkyl, 5- to 7-membered heterocycloalkyl, (C2-C30)alkenyl, (C2-C30)alkynyl, (C6-C30)aryl, (C3-C30)heteroaryl, mono- or di(C1-C30)alkylamino, mono- or di(C6-C30)arylamino, R^{a}R^{b}R^{c}Si— [wherein R^{a}, R^{b} and R^{c} independently represent (C1-C30)alkyl or (C6-C30)aryl], R^{d}Y— [wherein Y represents O or S, and R^{d} represents (C1-C30)alkyl or (C6-C30)aryl], mono- or di(C6-C30)arylboranyl, mono- or di(C1-C60)alkylboranyl, nitro or hydroxyl;

the alkyl, cycloalkyl, heterocycloalkyl, alkenyl, alkynyl, aryl, heteroaryl, alkylamino, arylamino, arylboranyl or alkylboranyl of R and R₁ through R₆ and the alkyl or aryl of R^{a}ᵣ R^{b}, R^{c} and R^{d} may be further substituted by one or more substituent(s) selected from a group consisting of (C1-C30)alkyl, halogen, cyano, (C3-C30)cycloalkyl, 5- to 7-membered heterocycloalkyl, (C2-C30)alkenyl, (C2-C30)alkynyl, (C6-C30)aryl, (C1-C30)alkoxy, (C6-C30)aryloxy, (C6-C30)aryl substituted by P(═O)R^{e}R^{f} [wherein R^{e} and R^{f} independently represent (C1-C30)alkyl or (C6-C30)aryl], (C3-C30)heteroaryl, (C3-C30)heteroaryl substituted by (C6-C30)aryl, (C3-C30)heteroaryl substituted by (C1-C30)alkyl, (C6-C30)ar(C1-C30)alkyl, (C6-C30)arylthio, (C1-C30)alkylthio, mono- or di(C1-C30)alkylamino, mono- or di(C6-C30)arylamino, tri(C1-C30)alkylsilyl, di(C1-C30)alkyl(C6-C30)arylsilyl, tri(C6-C30)arylsilyl, mono- or di(C6-C30)arylboranyl, mono- or di(C1-C30)alkylboranyl, nitro and hydroxyl; and

the heterocycloalkyl or heteroaryl may contain one or more heteroatom(s) selected from B, N, O, S, P(═O), Si and P.

In the present invention, the alkyl moiety of “(C1-C30)alkyl, tri(C1-C30)alkylsilyl, di(C1-C30)alkyl(C6-C30)arylsilyl, (C6-C30)ar(C1-C30)alkyl, (C1-C30)alkyloxy, (C1-C30)alkylthio, or the like may have 1 to 20 carbon atoms, more specifically 1 to 10 carbon atoms. The aryl moiety of “(C6-C30)aryl, di(C1-C30)alkyl(C6-C30)arylsilyl, tri(C6-C30)arylsilyl, (C6-C30)ar(C1-C30)alkyl, (C6-C30)aryloxy, (C6-C30)arylthio, or the like may have 6 to 20 carbon atoms, more specifically 6 to 12 carbon atoms. The heteroaryl of “(C3-C30)heteroaryl” may have 4 to 20 carbon atoms, more specifically 4 to 12 carbon atoms. The cycloalkyl of “(C3-C30)cycloalkyl” may have 3 to 20 carbon atoms, more specifically 3 to 7 carbon atoms. The alkenyl or alkynyl of “(C2-C30)alkenyl or alkynyl” may have 2 to 20 carbon atoms, more specifically 2 to 10 carbon atoms.

In the present invention, the alkyl includes linear or branched saturated monovalent hydrocarbon radical formed of only carbon atoms and hydrogen atoms or a combination thereof. The cycloalkyl includes hydrocarbon such as adamantyl or bicycloalkyl of a polycyclic ring as well as a monocyclic ring.

In the present invention, “aryl” means an organic radical derived from an aromatic hydrocarbon by the removal of one hydrogen atom, and may include a 4- to 7-membered, particularly 5- or 6-membered, single ring or fused ring, including a plurality of aryls linked by single bond(s). Specific examples include phenyl, naphthyl, biphenyl, anthryl, indenyl, fluorenyl, phenanthryl, triphenylenyl, pyrenyl, perylenyl, chrysenyl, naphthacenyl, fluoranthenyl, etc., but are not limited thereto. The naphthyl includes 1-naphthyl and 2-naphthyl, the anthryl includes 1-anthryl, 2-anthryl and 9-anthryl, and the fluorenyl includes 1-fluorenyl, 2-fluorenyl, 3-fluorenyl, 4-fluorenyl and 9-fluorenyl. In the present invention, “heteroaryl” means an aryl group containing 1 to 4 heteroatom(s) selected from B, N, O, S, P(═O), Si and P as aromatic ring backbone atom(s), other remaining aromatic ring backbone atoms being carbon. It may be 5- or 6-membered monocyclic heteroaryl or polycyclic heteroaryl resulting from condensation with a benzene ring, and may be partially saturated. Further, the heteroaryl includes more than one heteroaryls linked by single bond(s). The heteroaryl includes a divalent aryl group wherein the heteroatom(s) in the ring may be oxidized or quaternized to form, for example, an N-oxide or a quaternary salt. Specific examples include monocyclic heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., polycyclic heteroaryl such as benzofuranyl, benzothiophenyl, isobenzofuranyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenanthridinyl, benzodioxolyl, etc., an N-oxide thereof (e.g., pyridyl N-oxide, quinolyl N-oxide, etc.), a quaternary salt thereof, etc., but are not limited thereto.

Also, the organic electroluminescent compound of the present invention may be exemplified as compounds having following structures.

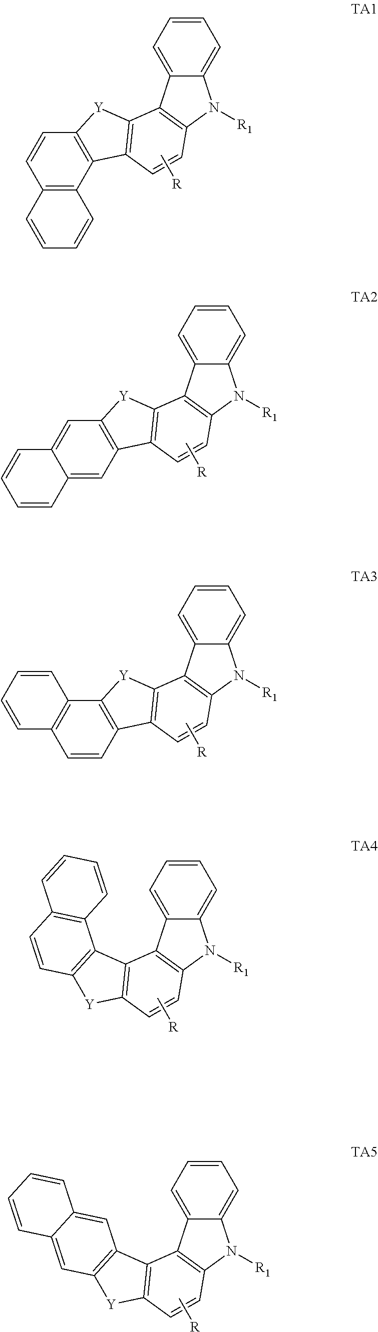

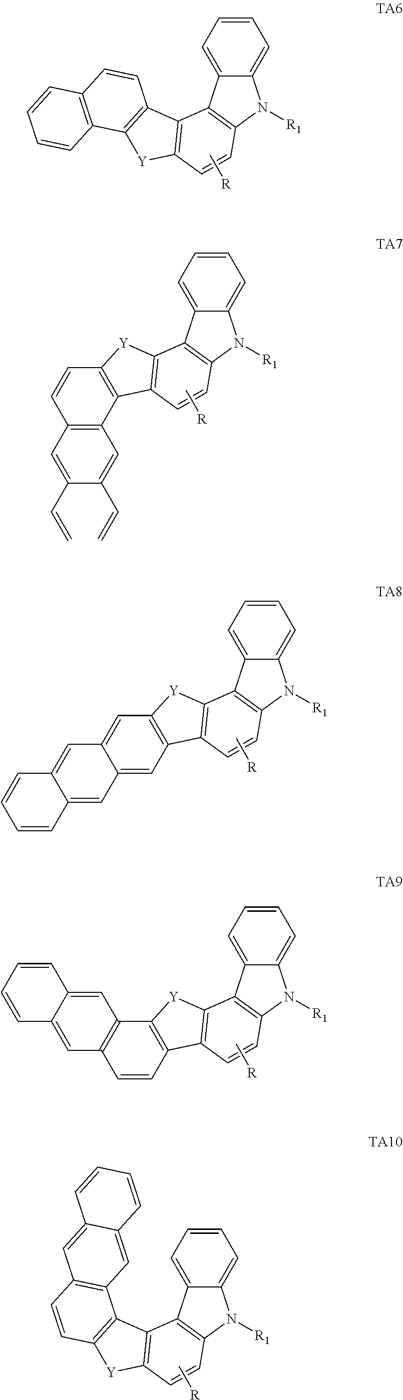

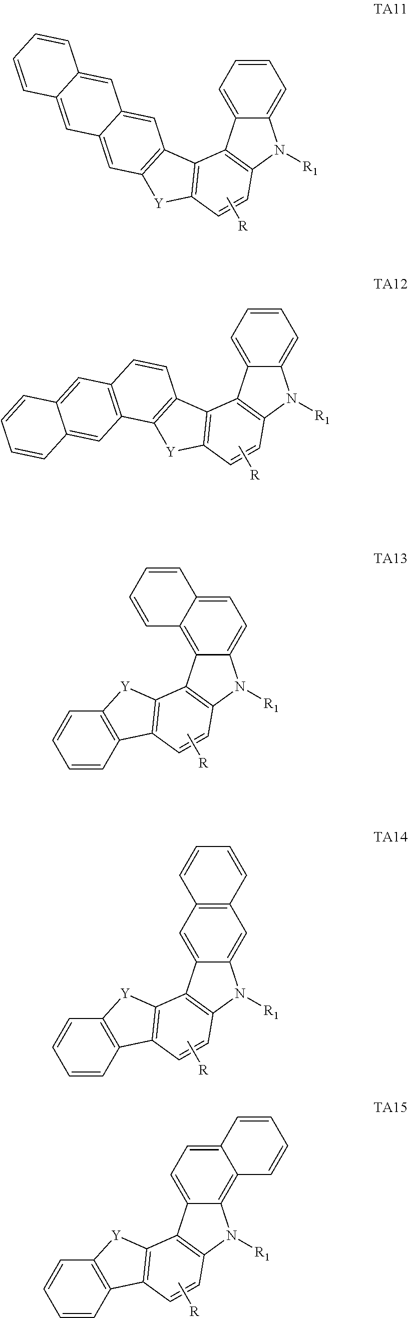

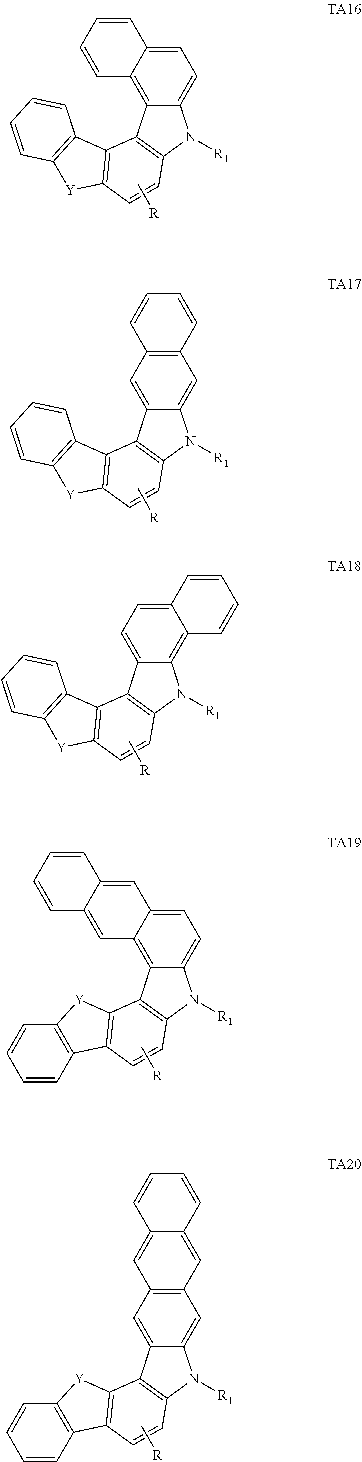

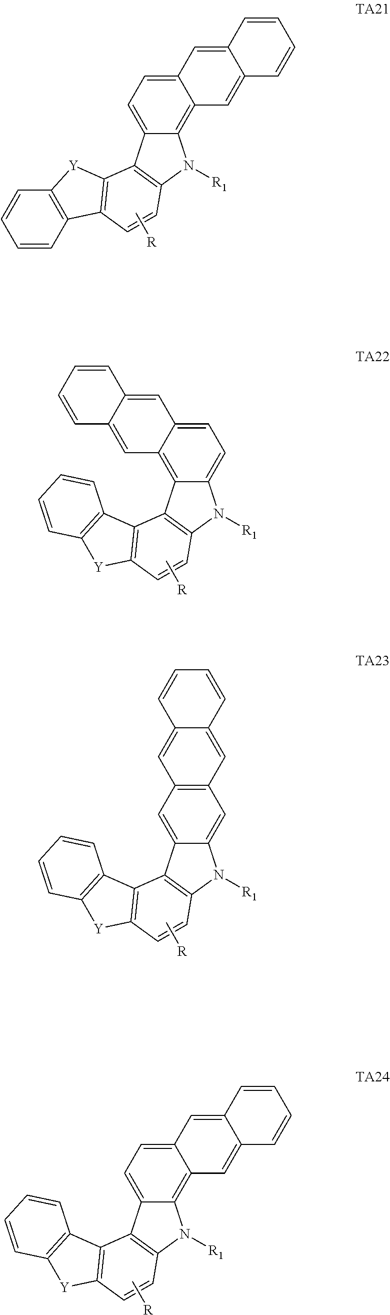

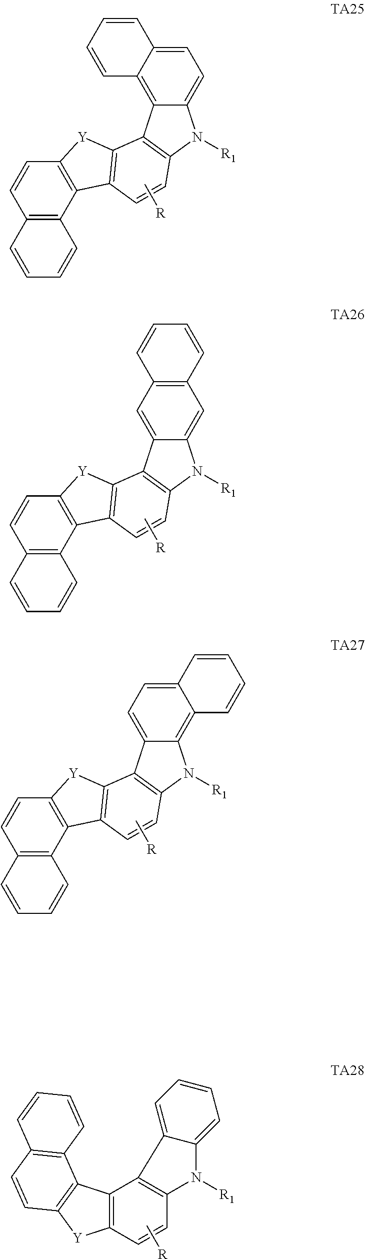

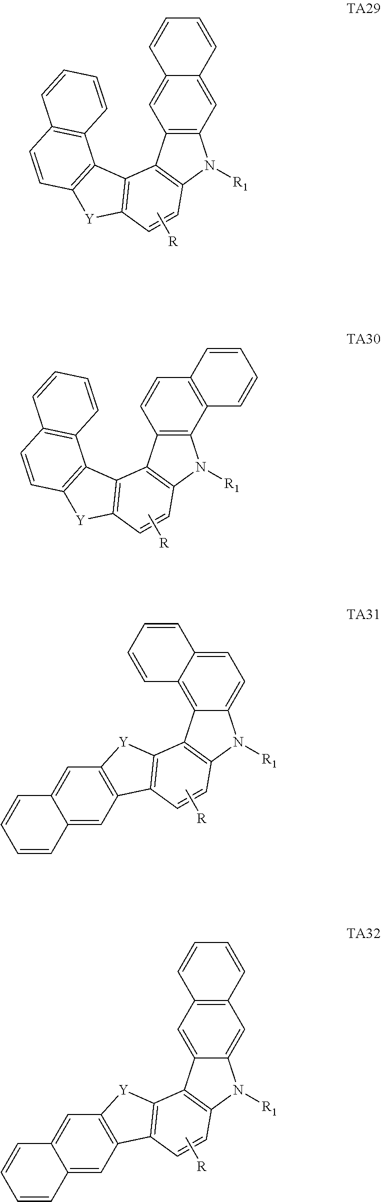

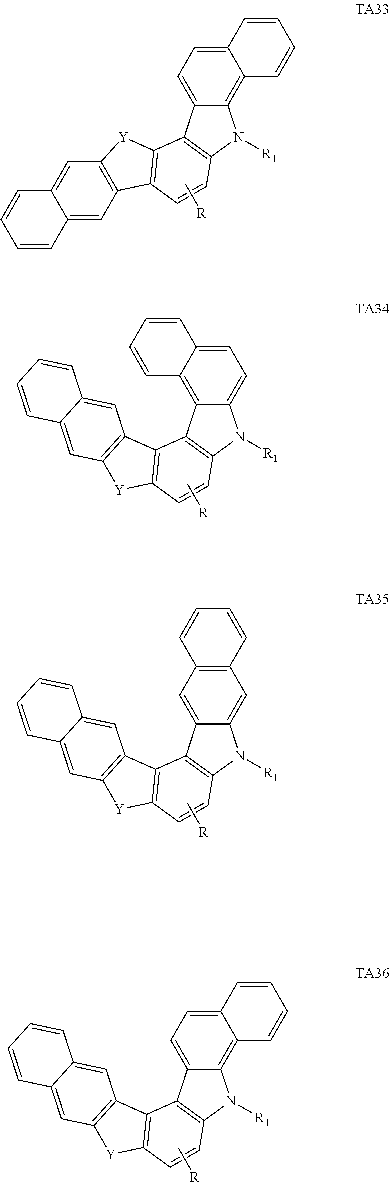

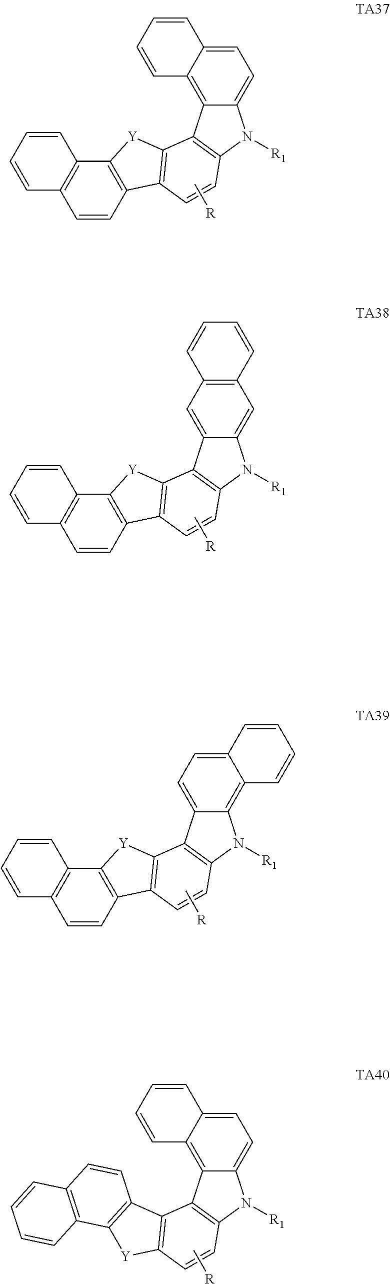

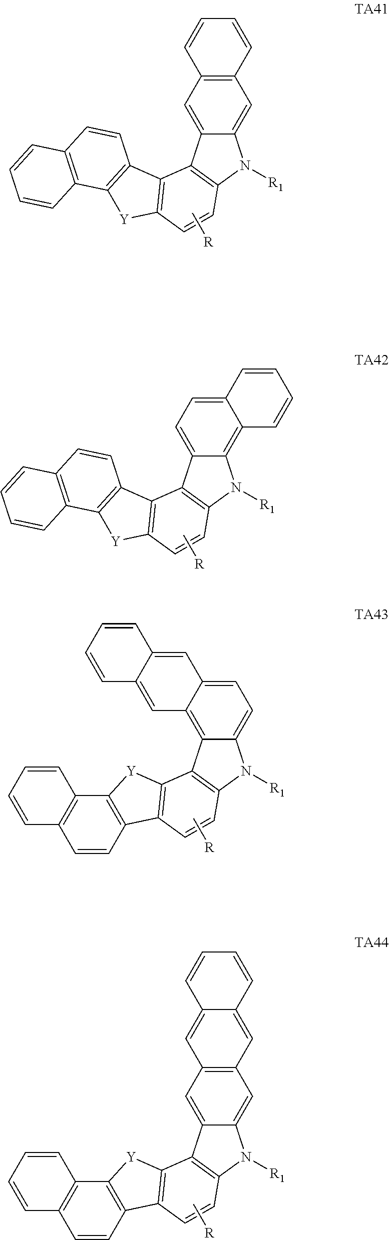

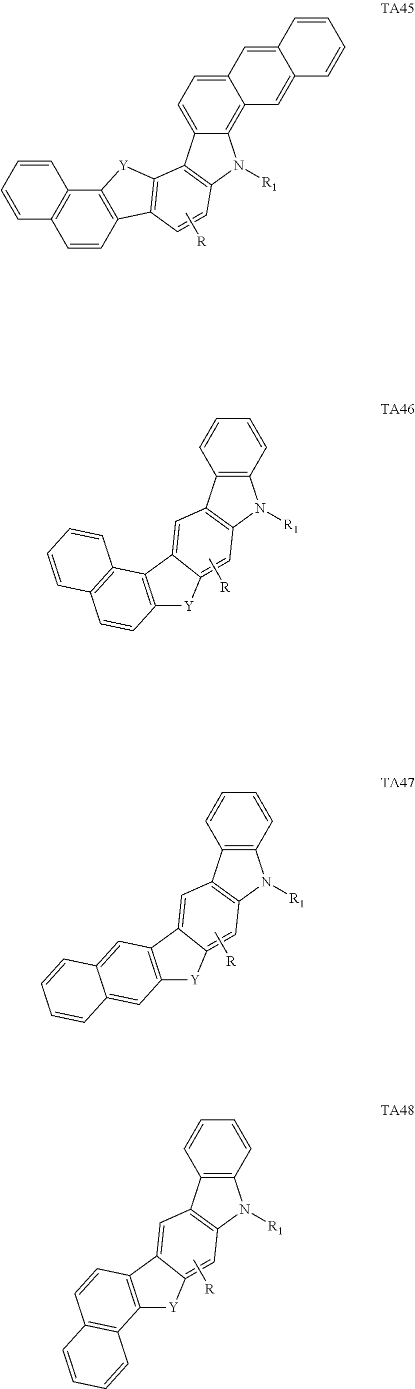

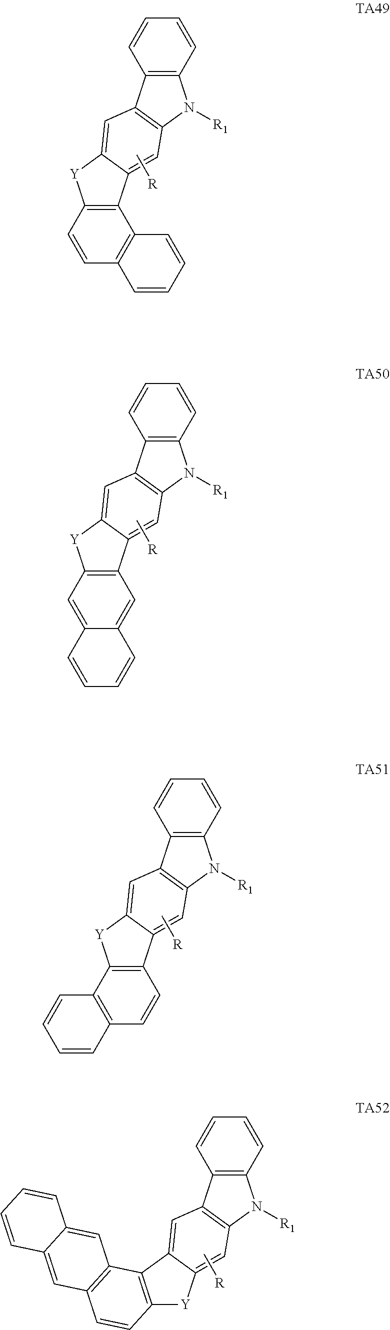

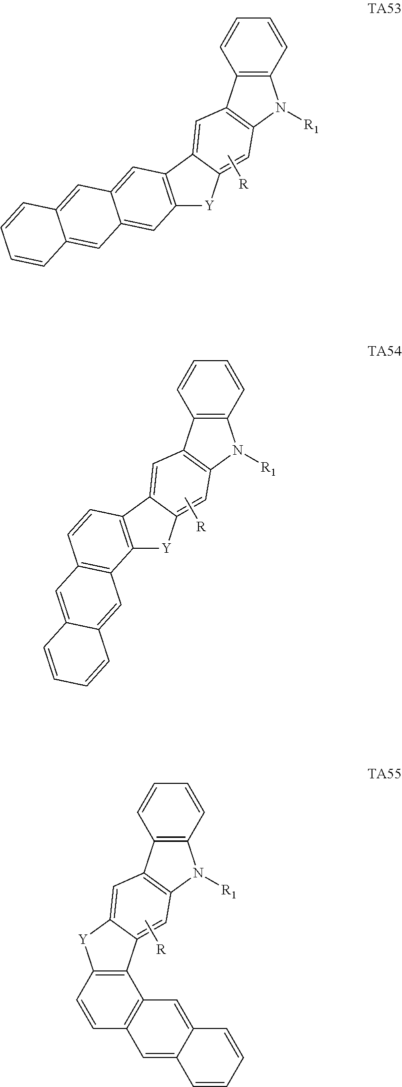

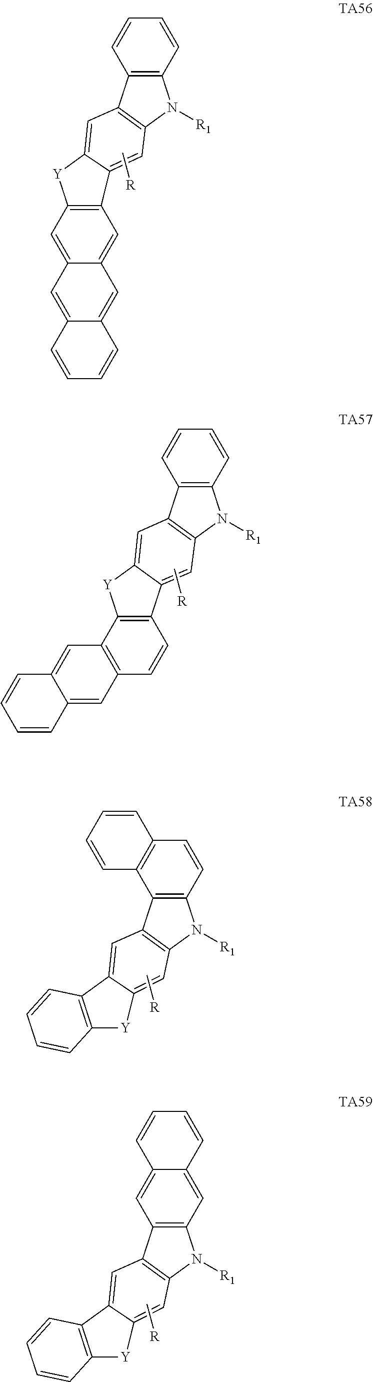

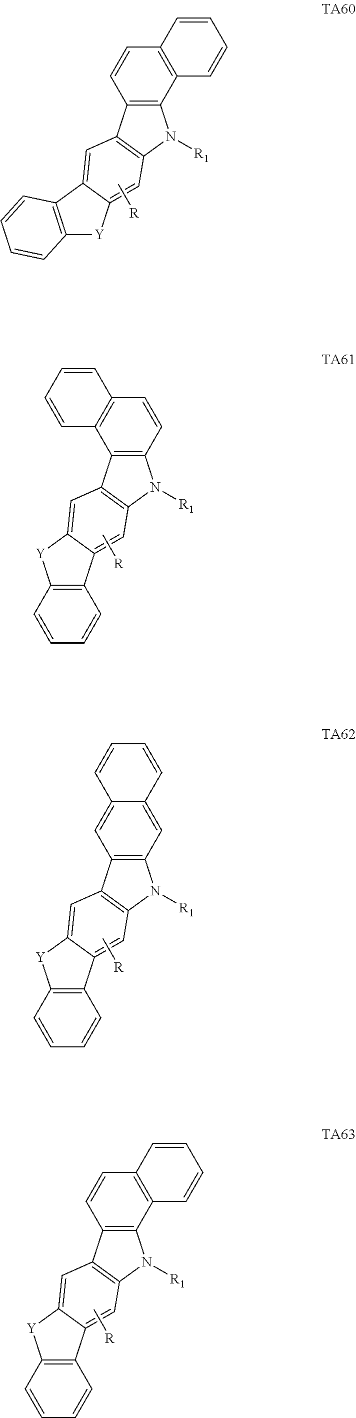

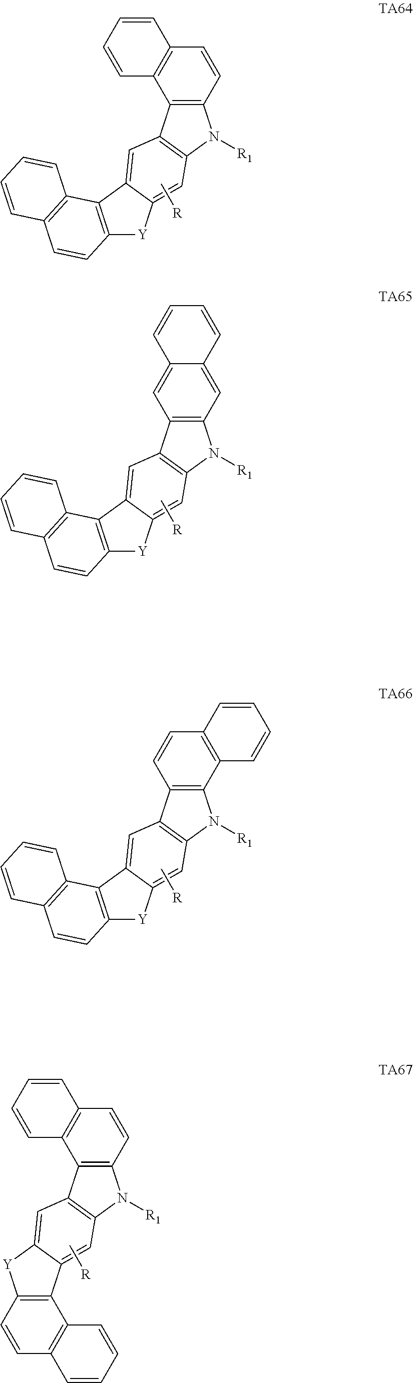

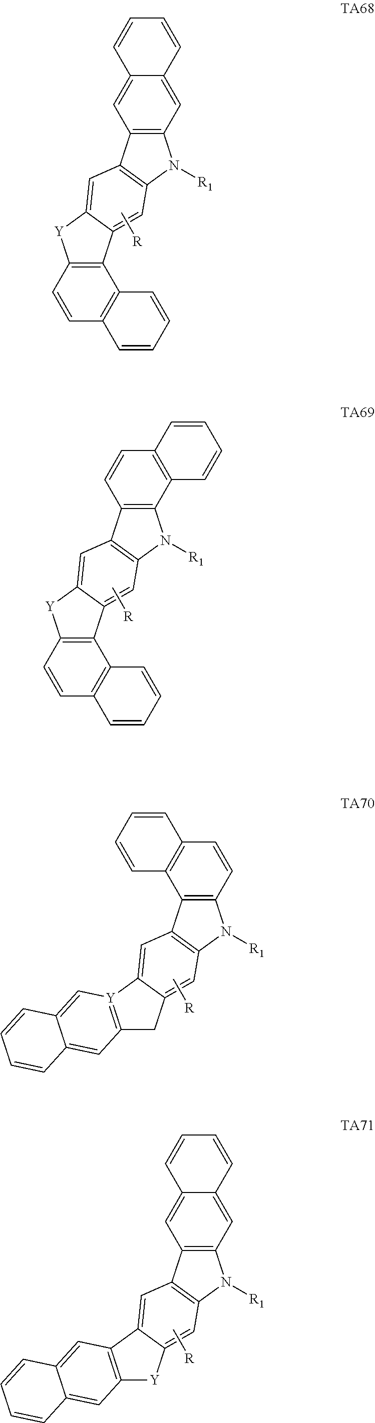

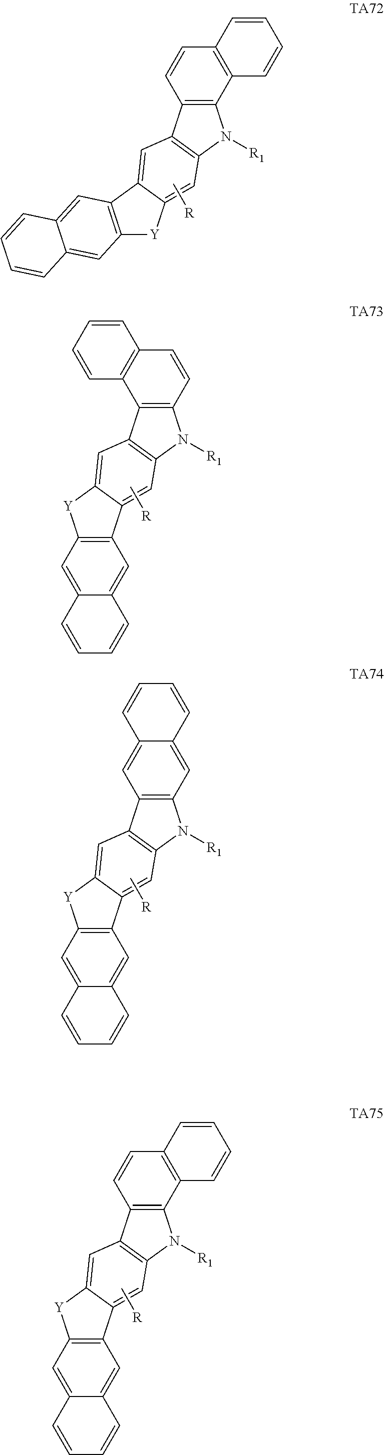

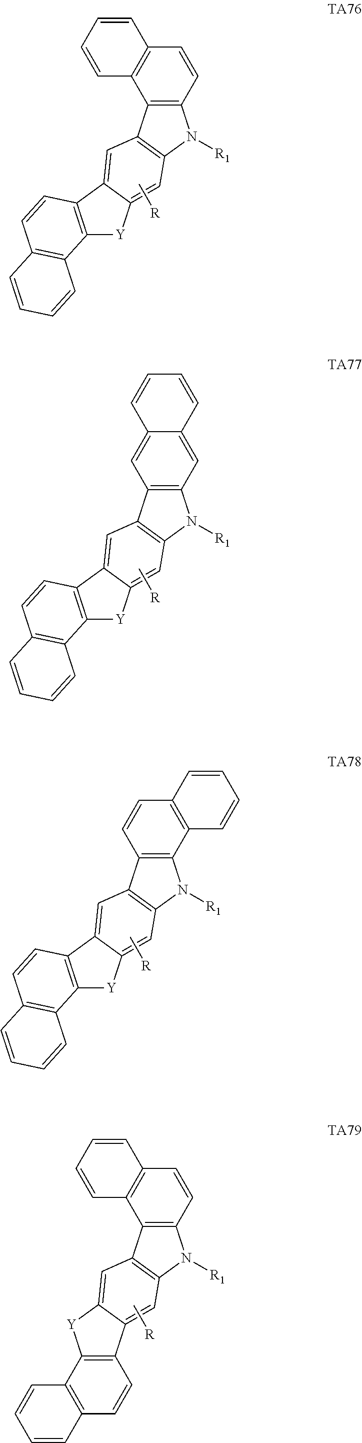

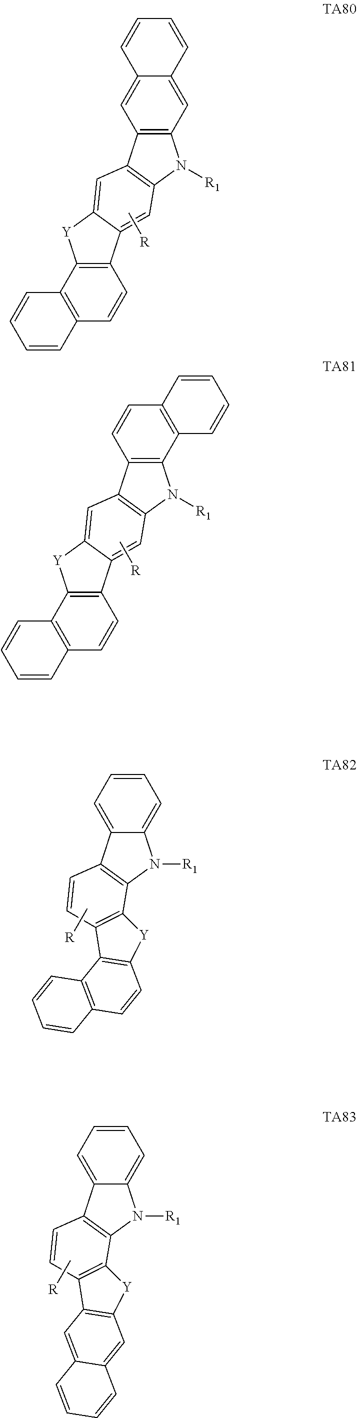

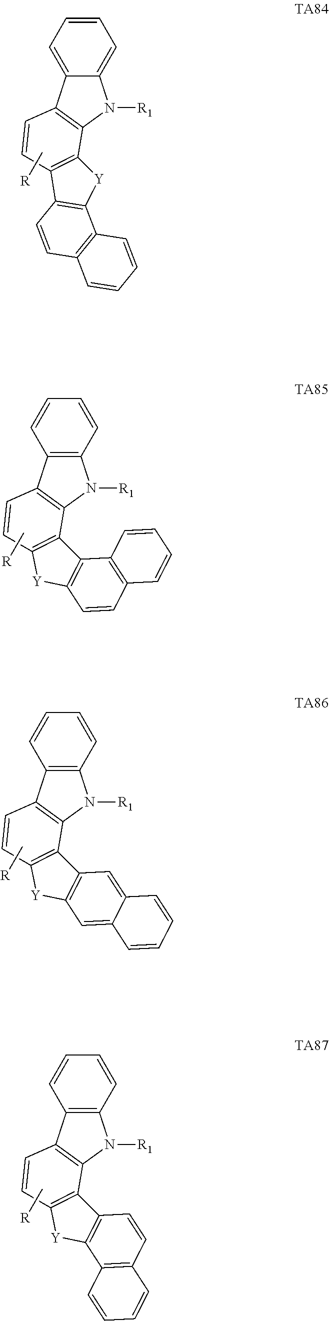

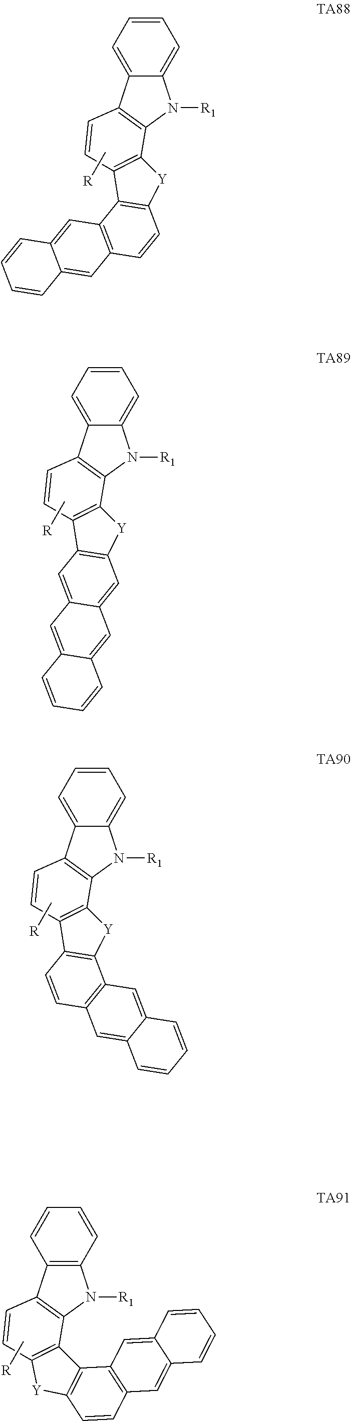

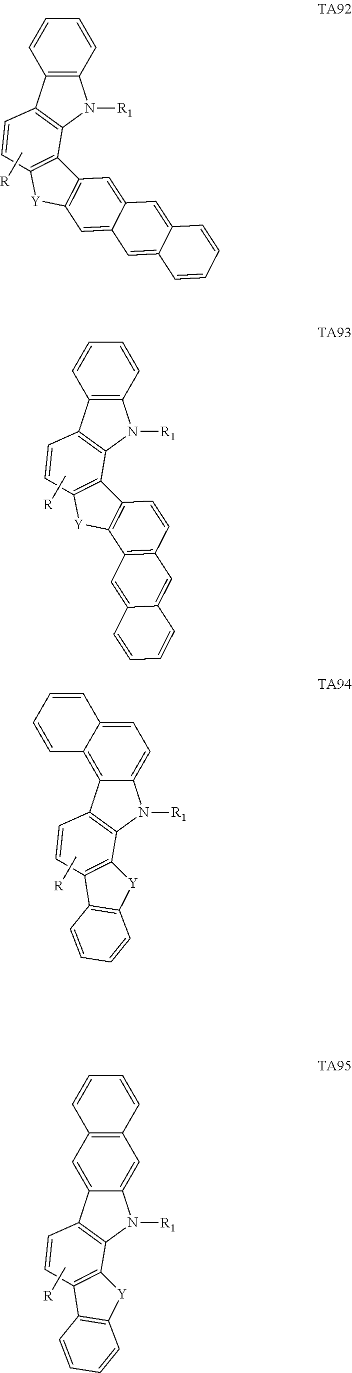

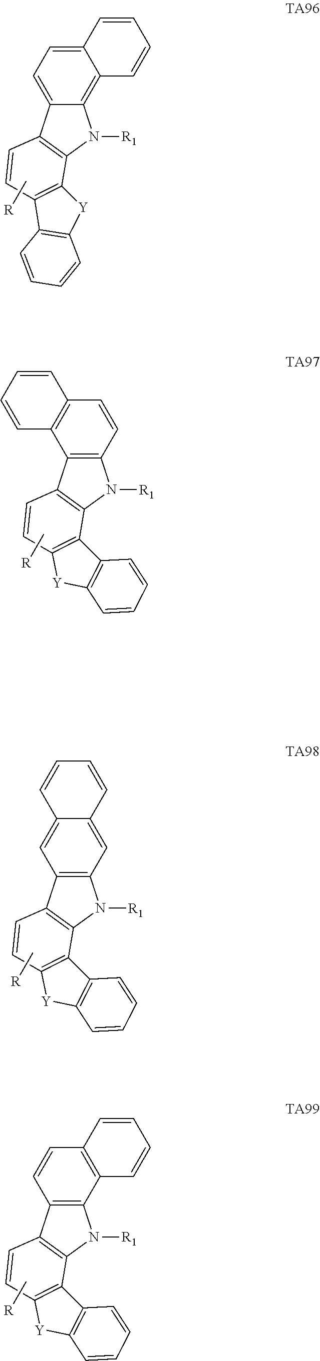

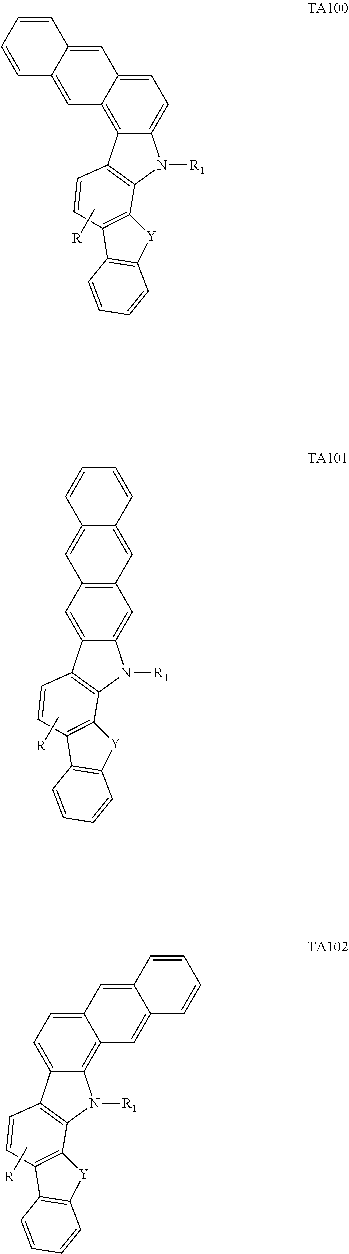

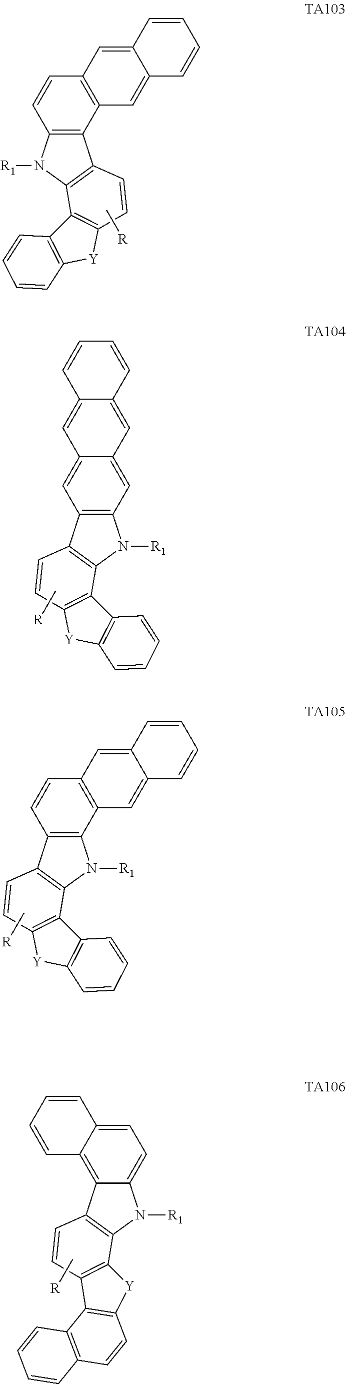

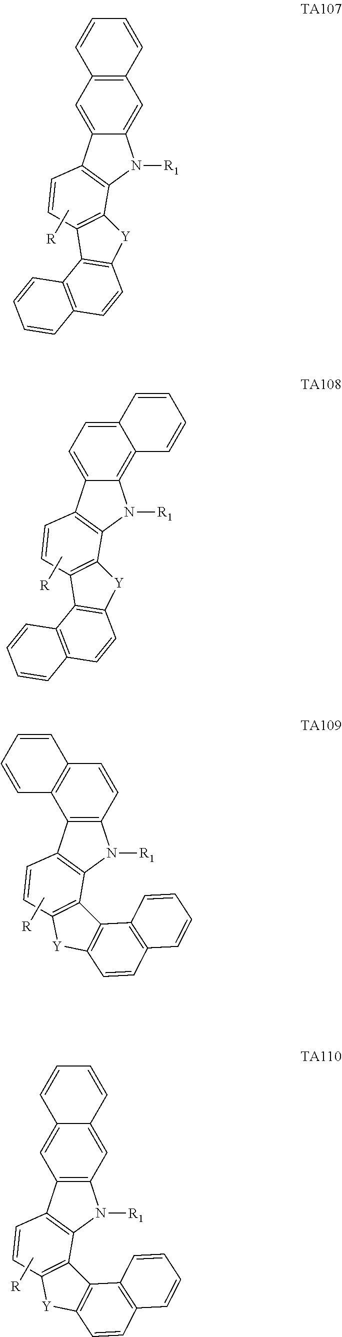

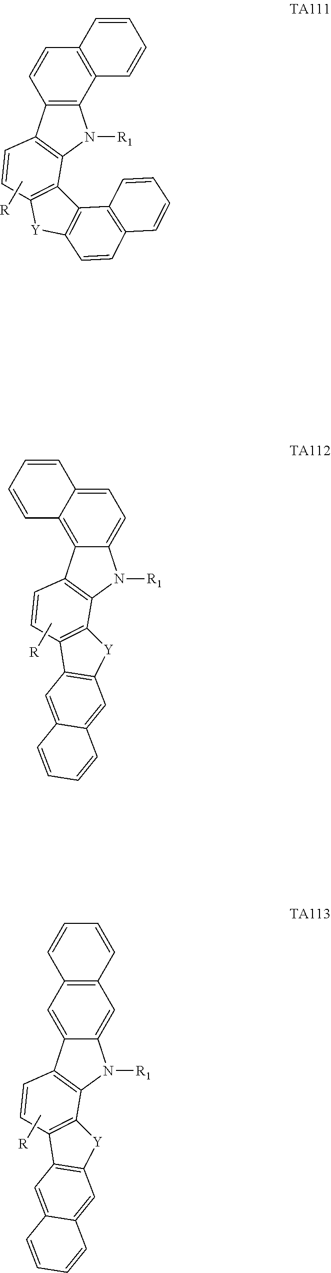

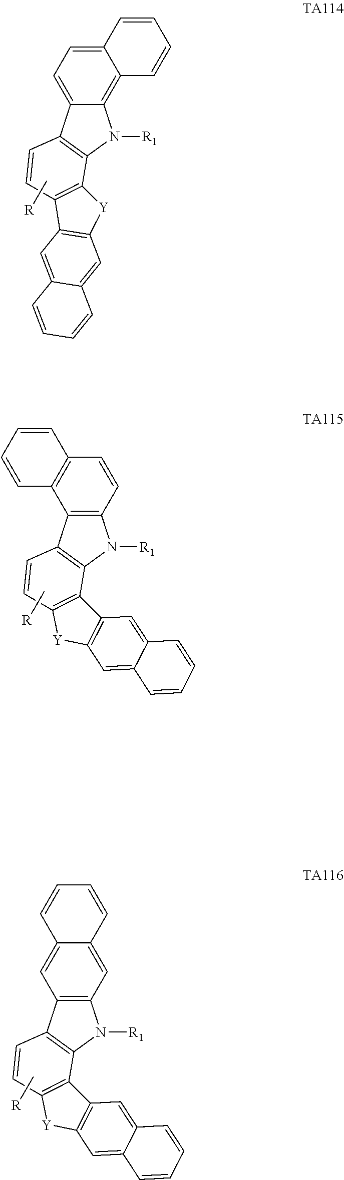

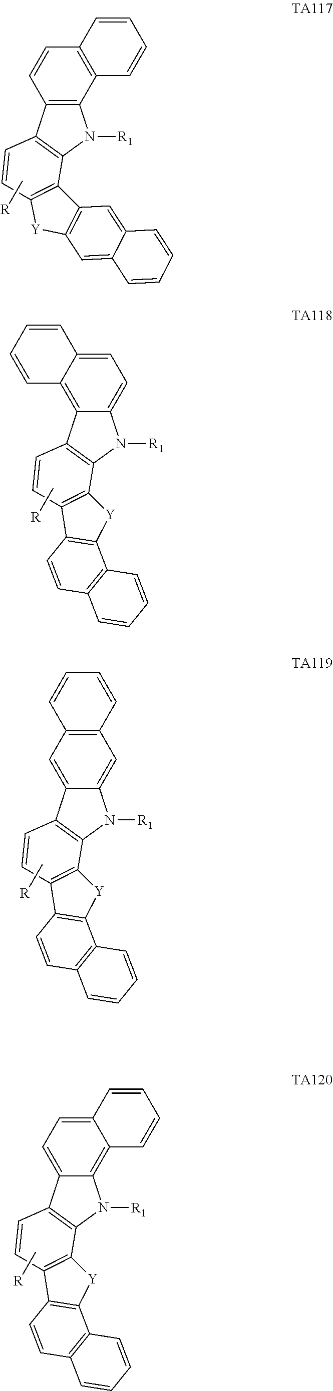

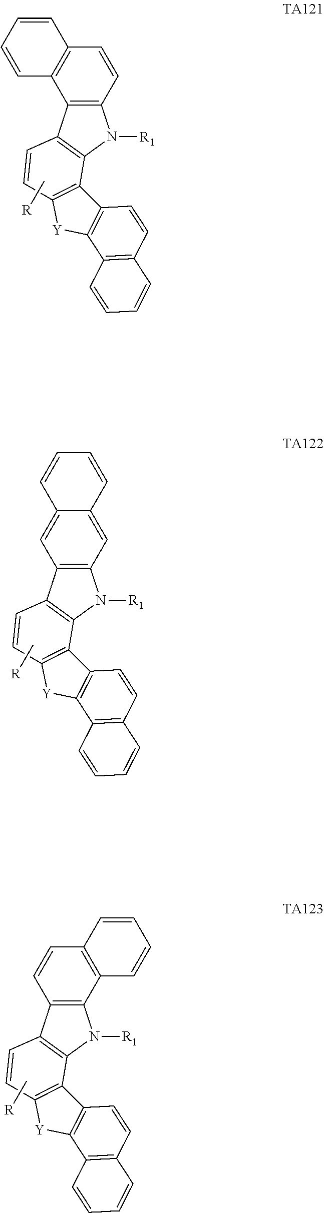

wherein,

Y represents C(R₂) (R₃) or Si (R₄) (R₅); and R and R₁ through R_{5 are the same as defined in the Chemical Formulas} 1 to 6.

Also, the organic electroluminescent compound of the present invention may be exemplified as compounds having following structures.

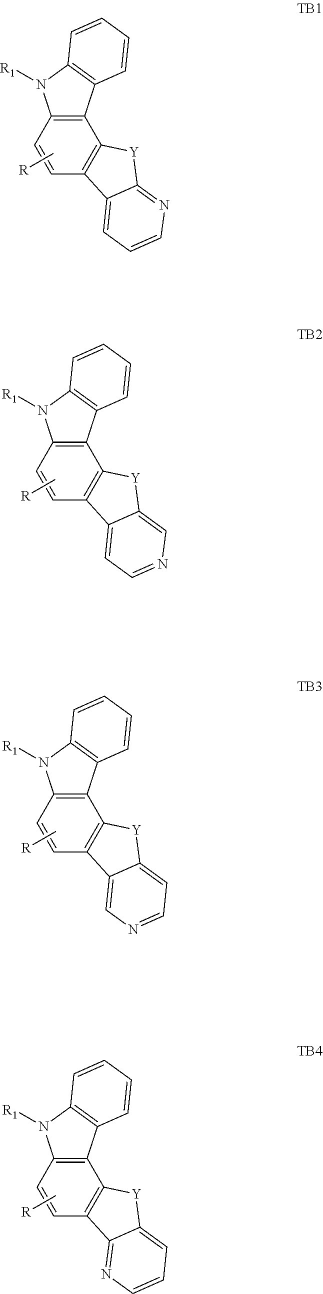

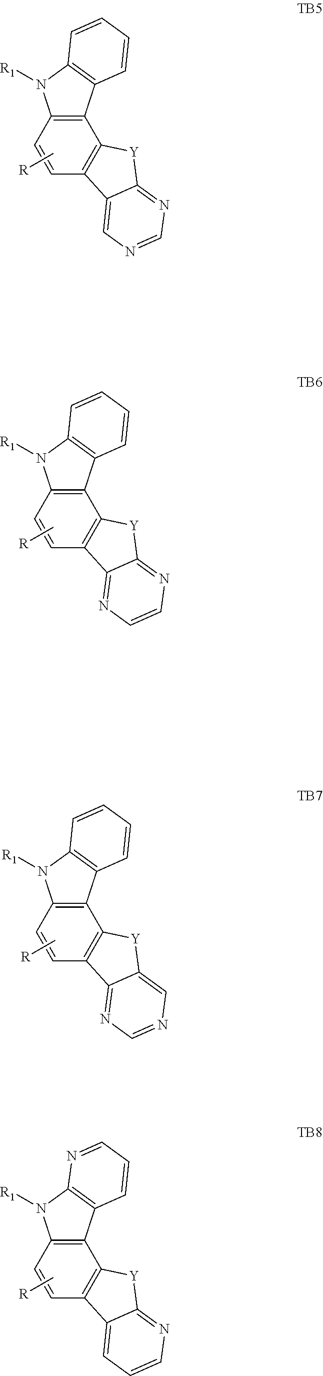

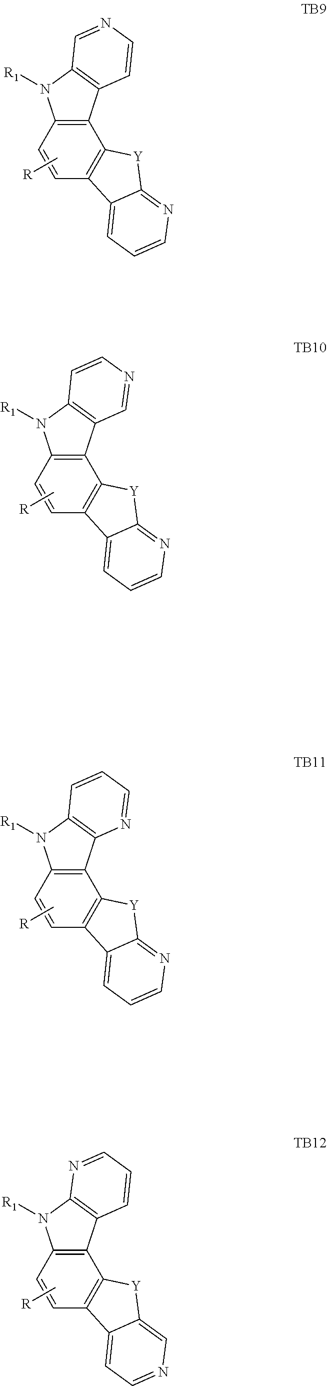

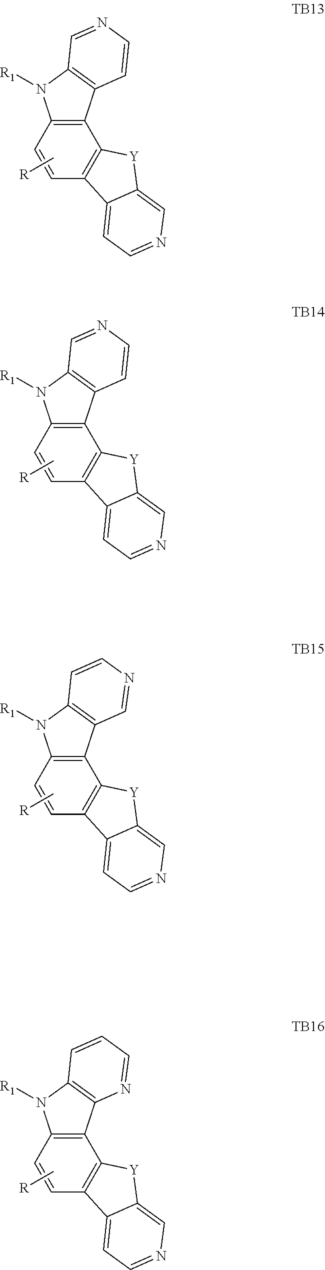

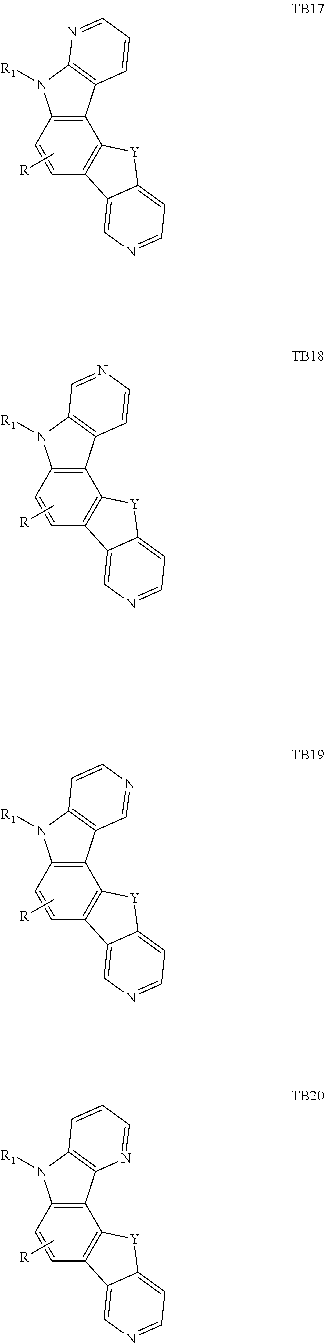

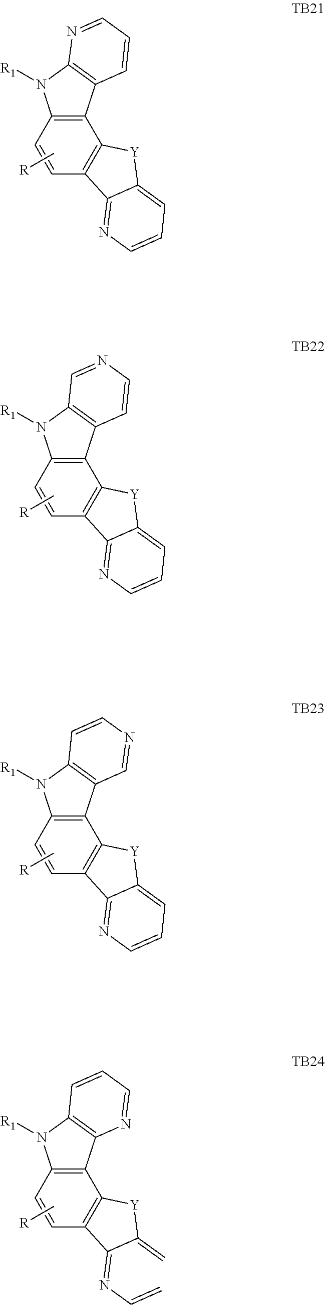

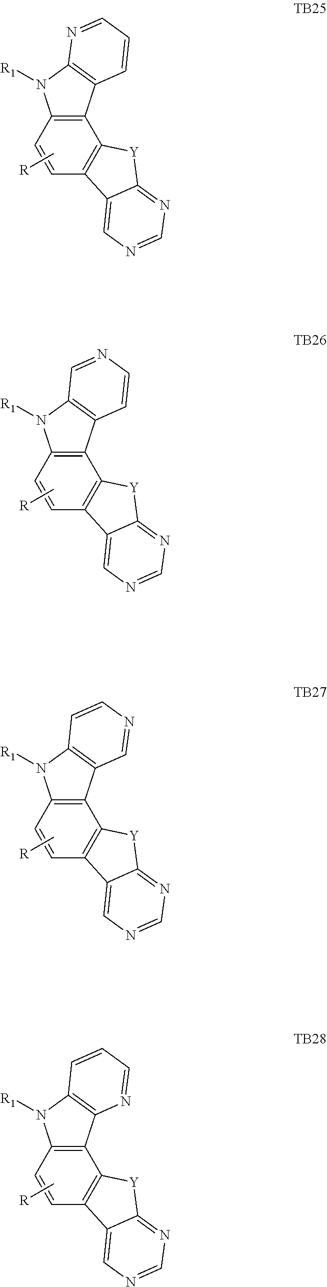

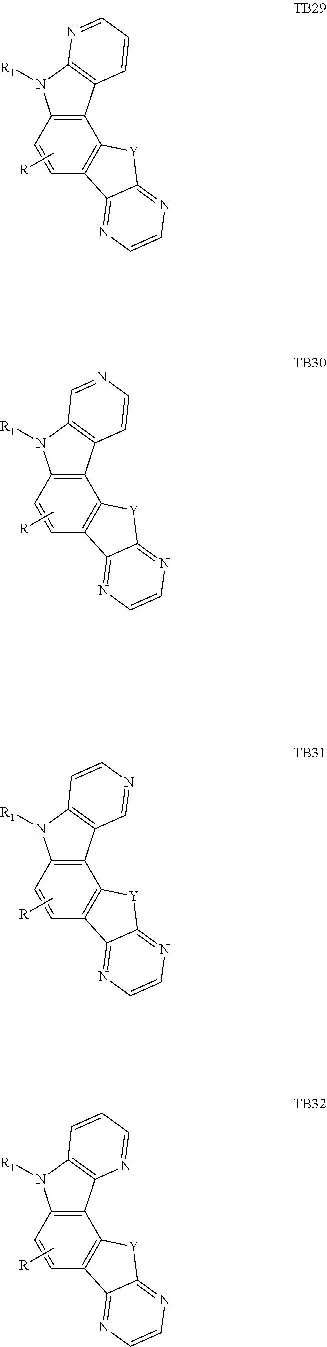

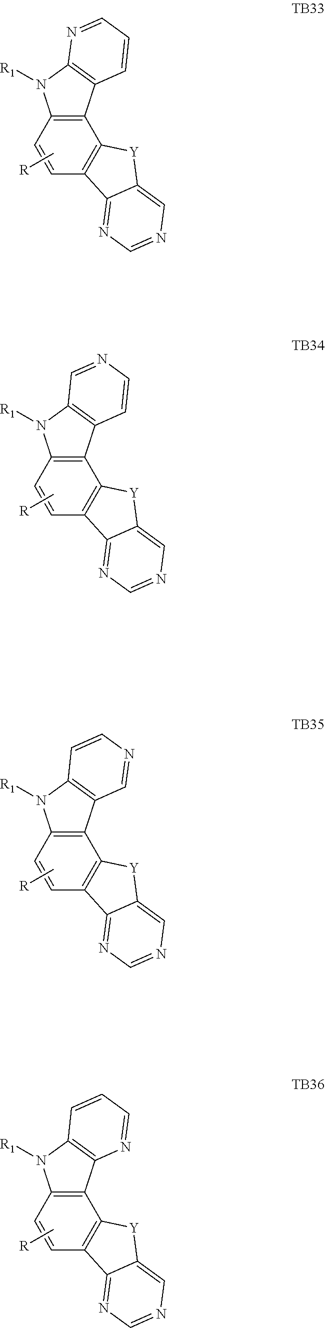

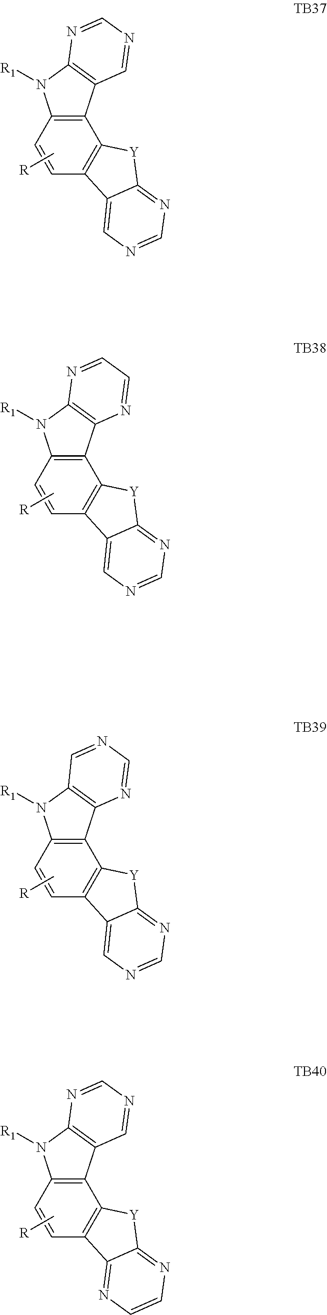

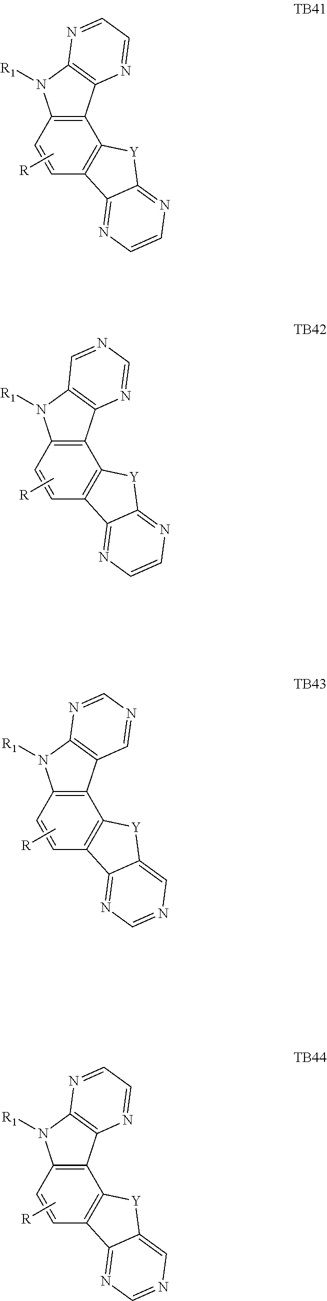

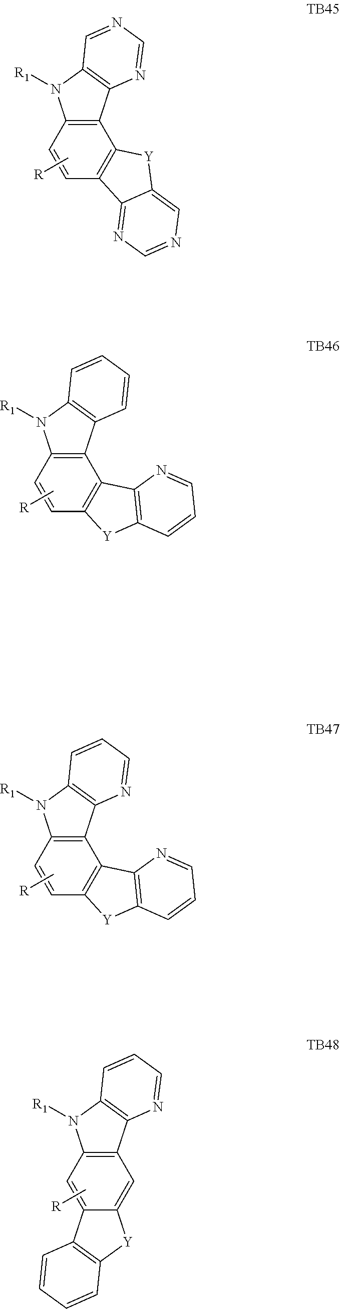

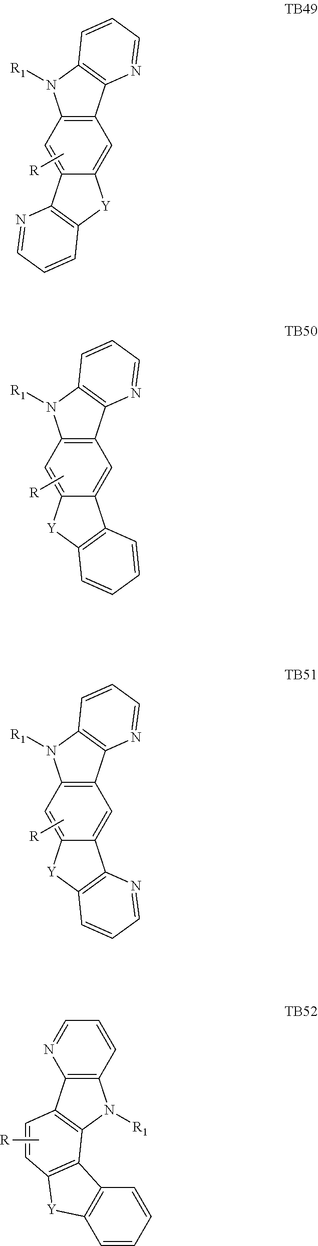

wherein,

Y represents C(R₂)(R₃) or Si(R₄)(R₅); and R and R₁ through R₅ are the same as defined in Chemical Formula 1 to 6.

In addition, the organic electroluminescent compound of the present invention may be exemplified as compounds having following structures.

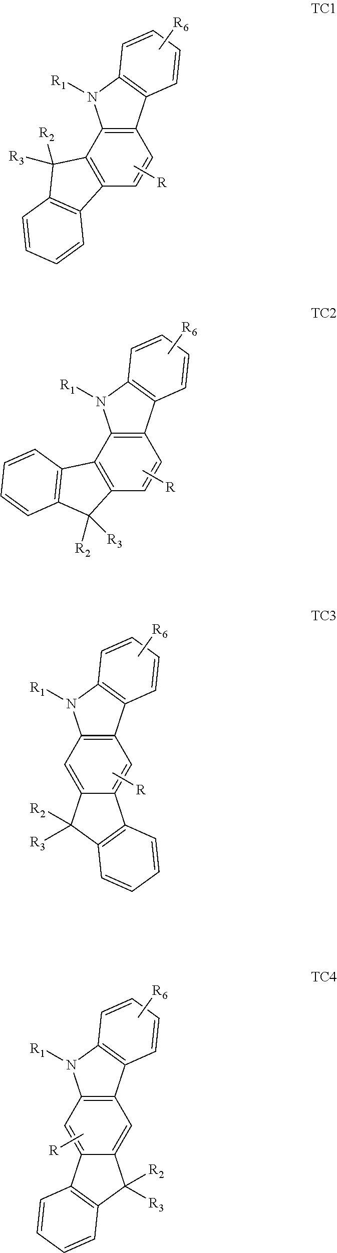

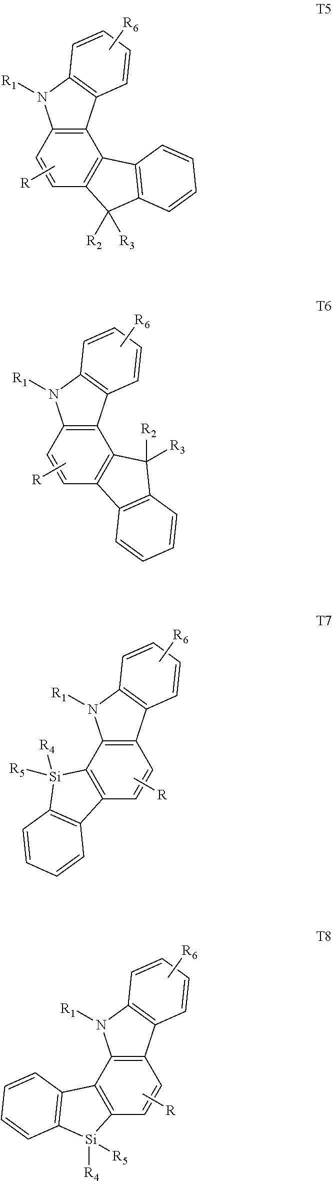

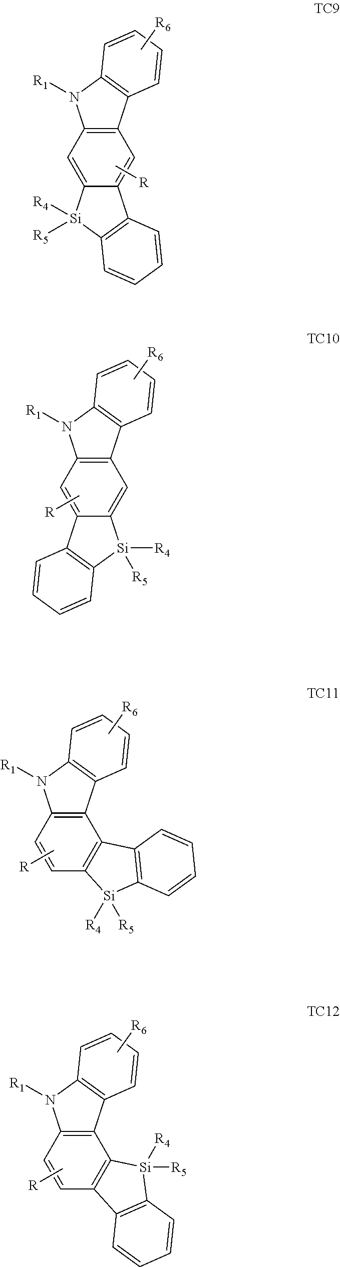

wherein,

R and R₁ through R₆ are the same as defined in Chemical Formulas 1 to 6.

To be specific, the R and R₂ through R₅ are independently selected from the group consisting of hydrogen, halogen, alkyl such as methyl, ethyl, propyl, butyl, pentyl, hexyl, ethylhexyl, heptyl, and octyl, and aryl such as phenyl, naphthyl, fluorenyl, biphenyl, phenanthryl, terphenyl, pyrenyl, perylenyl, spirobifluorenyl, fluoranthenyl, crycenyl, and triphenylenyl but are not limited thereto.

To be specific, the R₁ and R₆ independently represent phenyl, 1-naphthyl, 2-naphthyl or substituents selected from following structures but are not limited thereto.

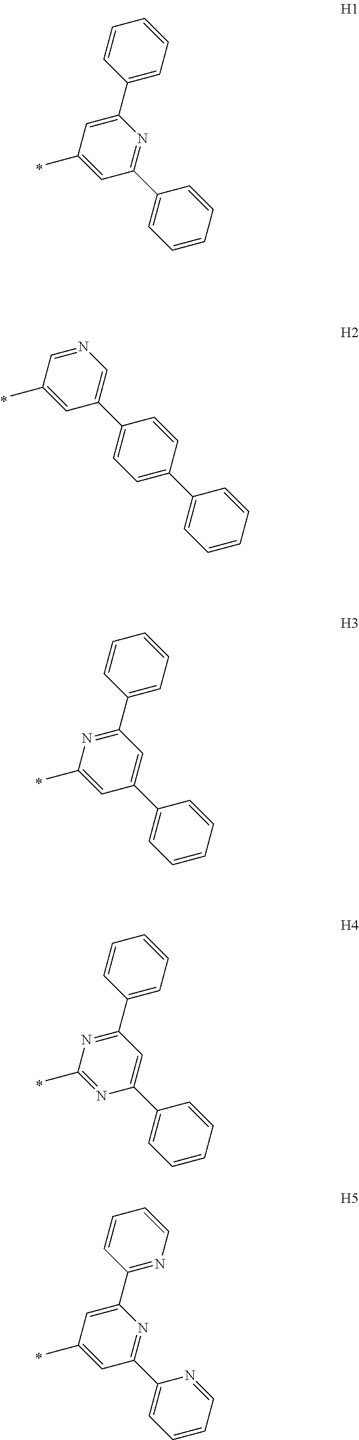

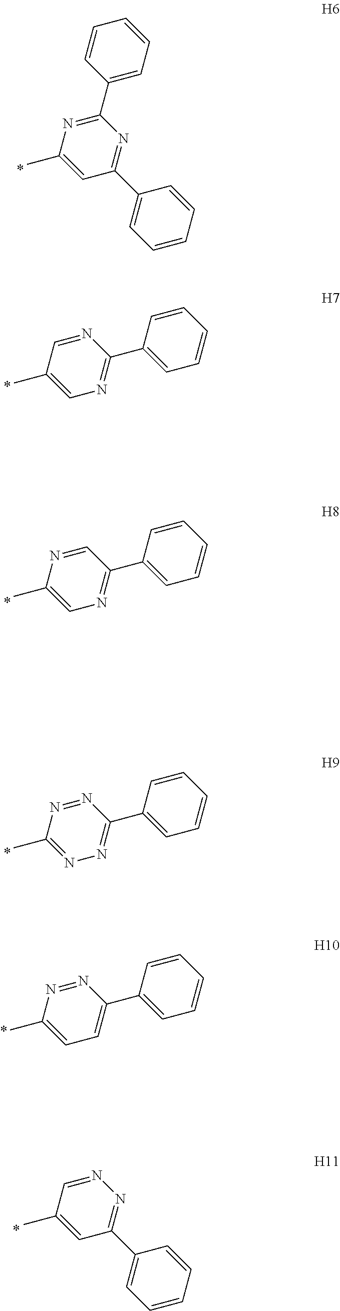

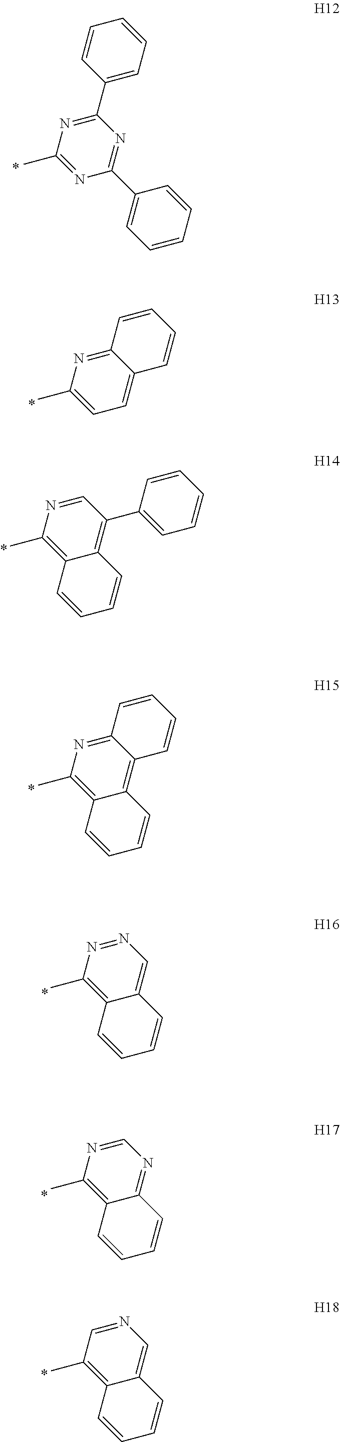

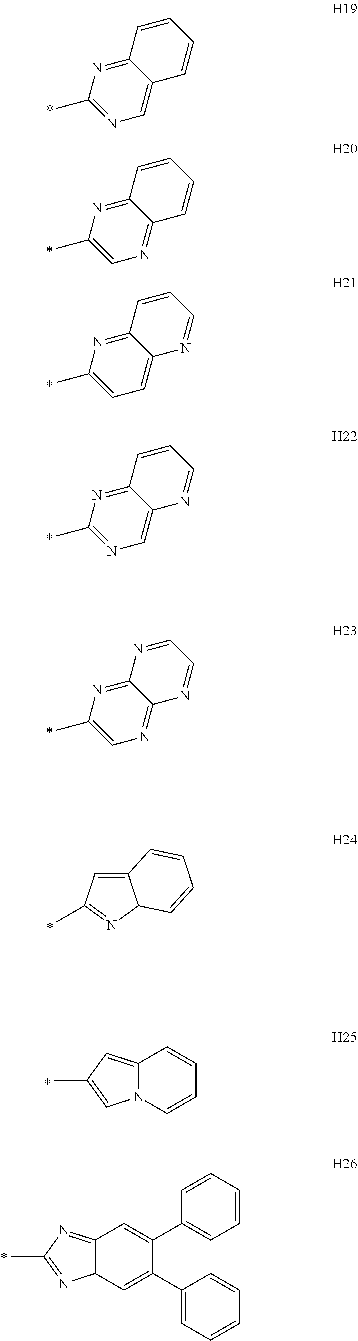

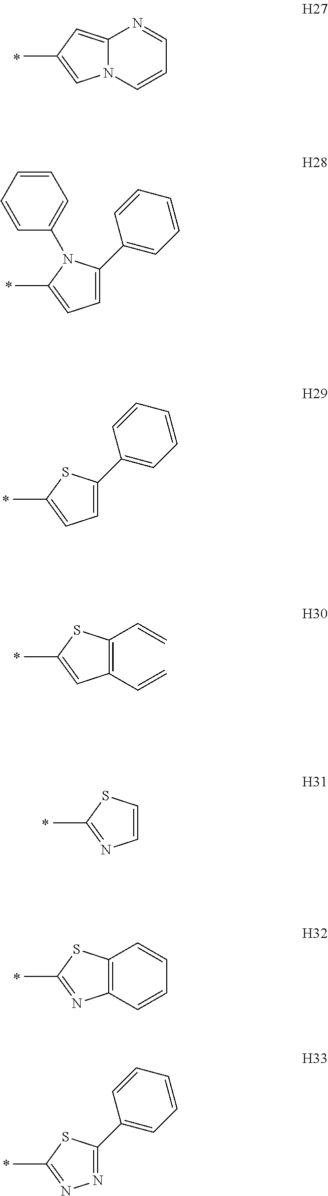

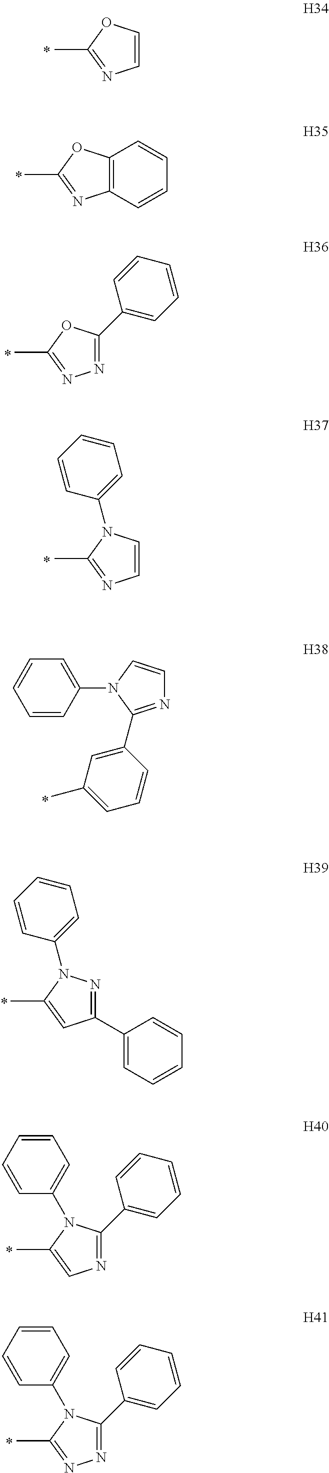

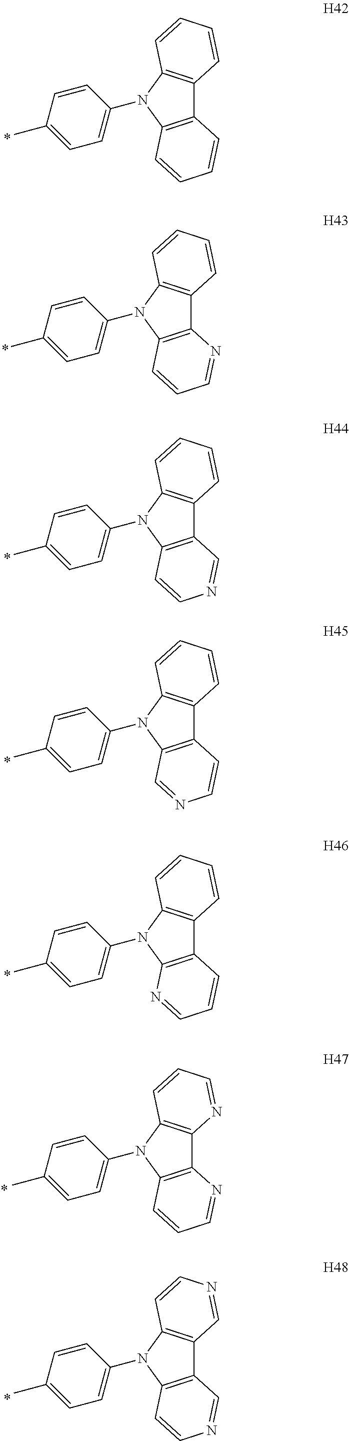

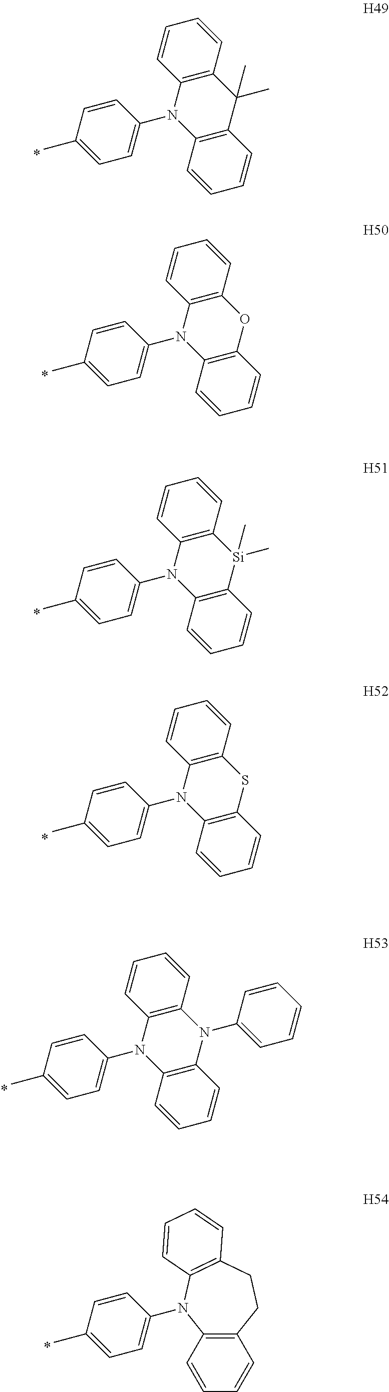

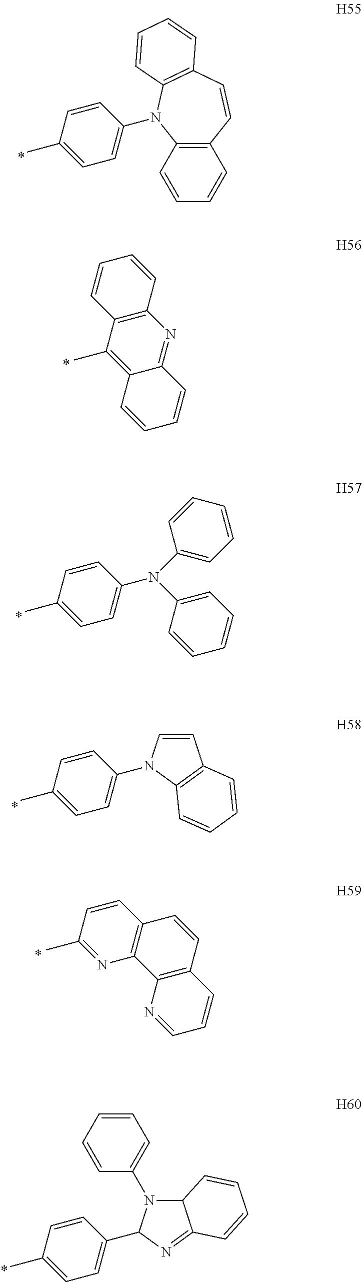

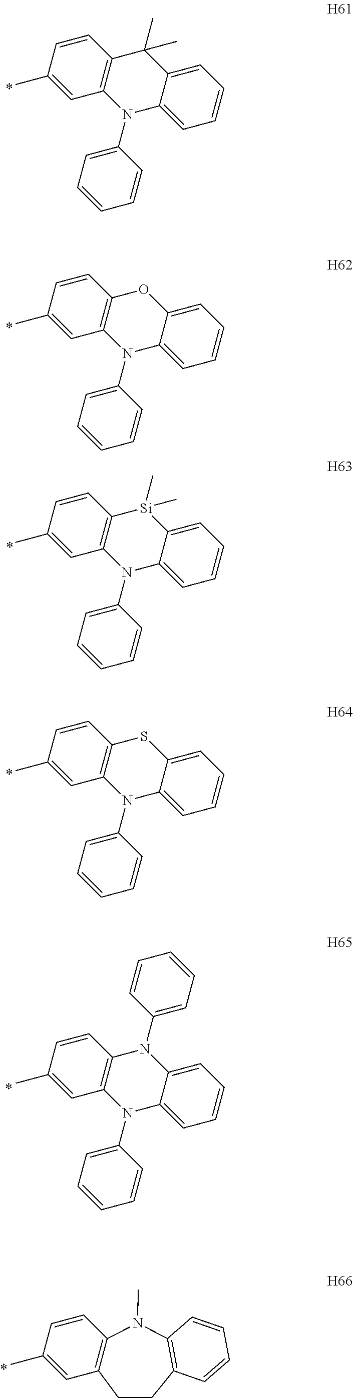

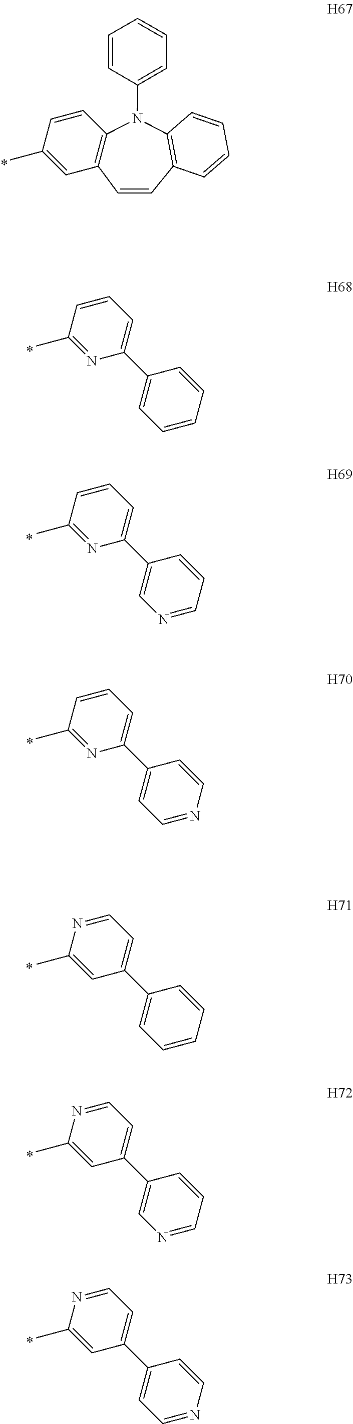

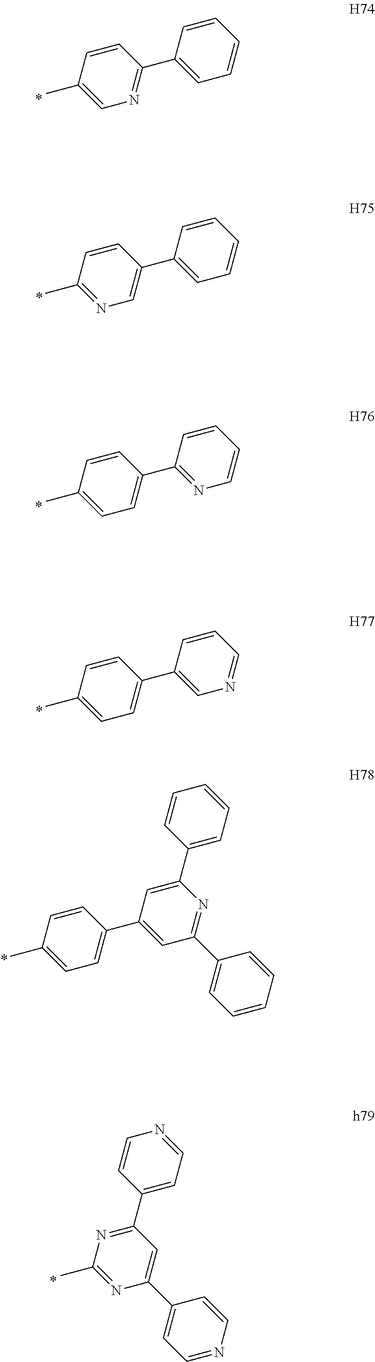

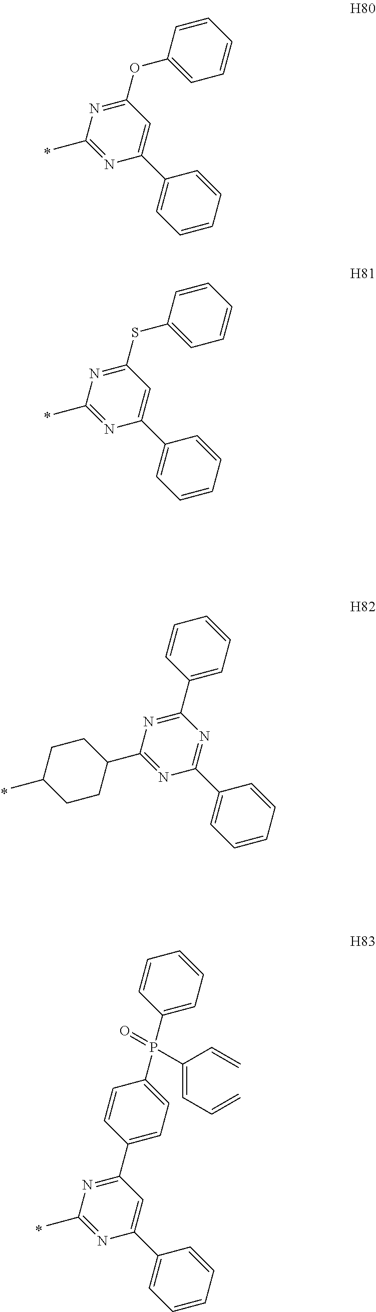

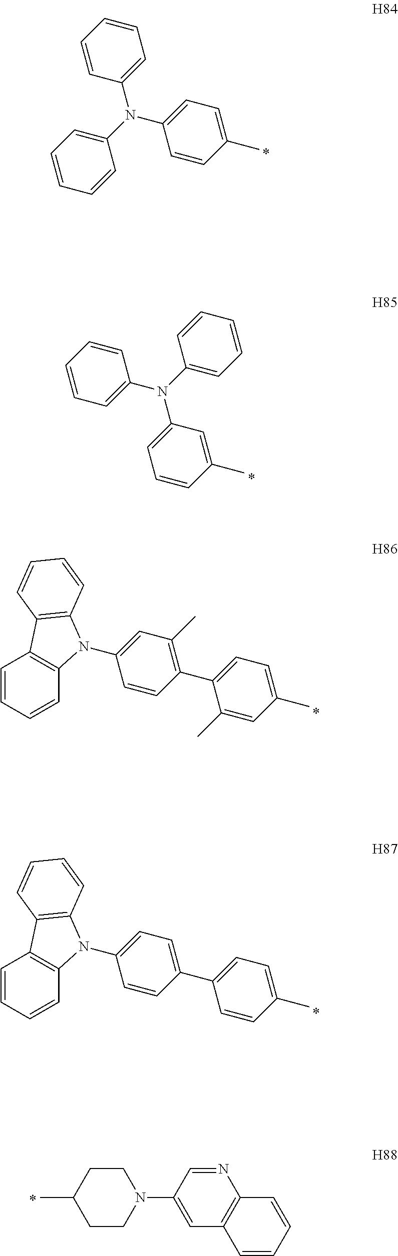

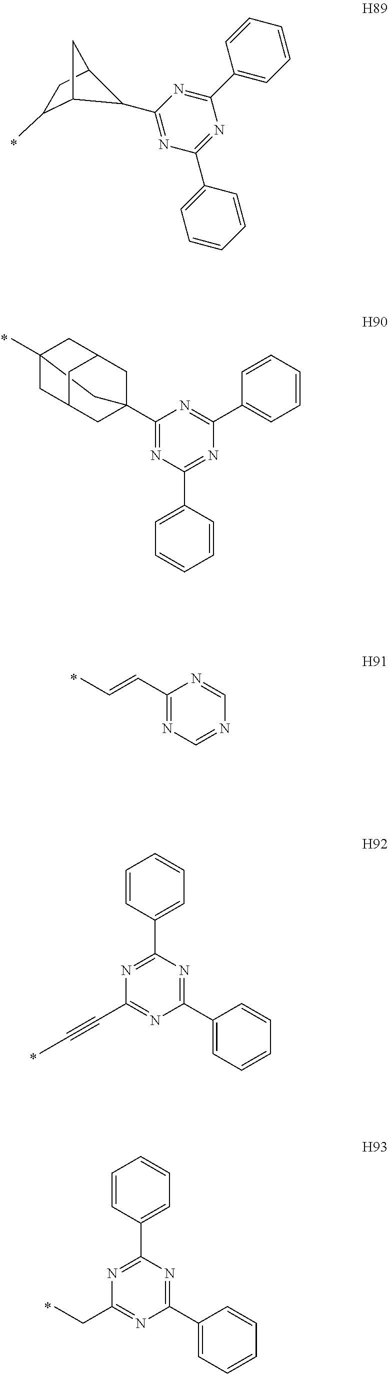

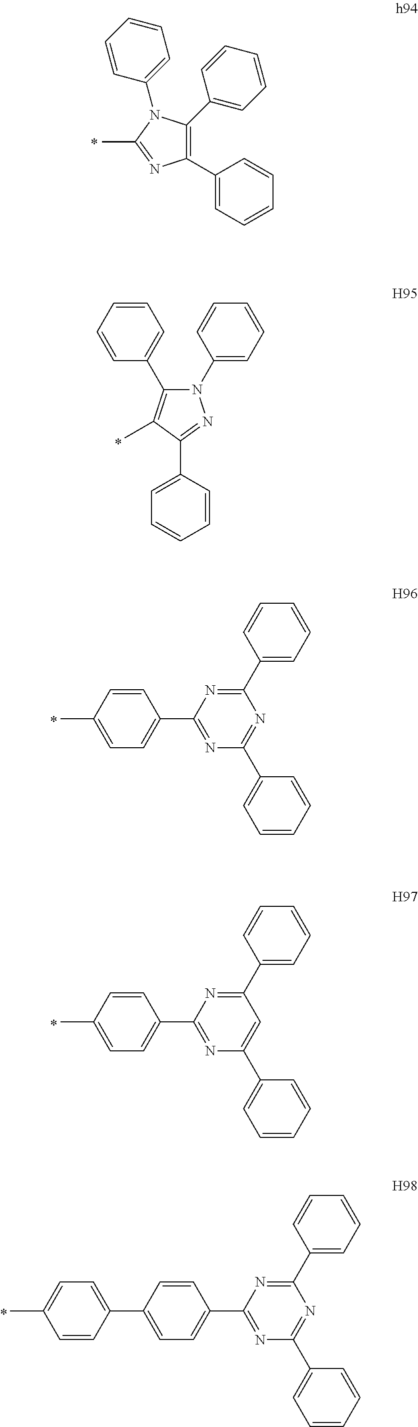

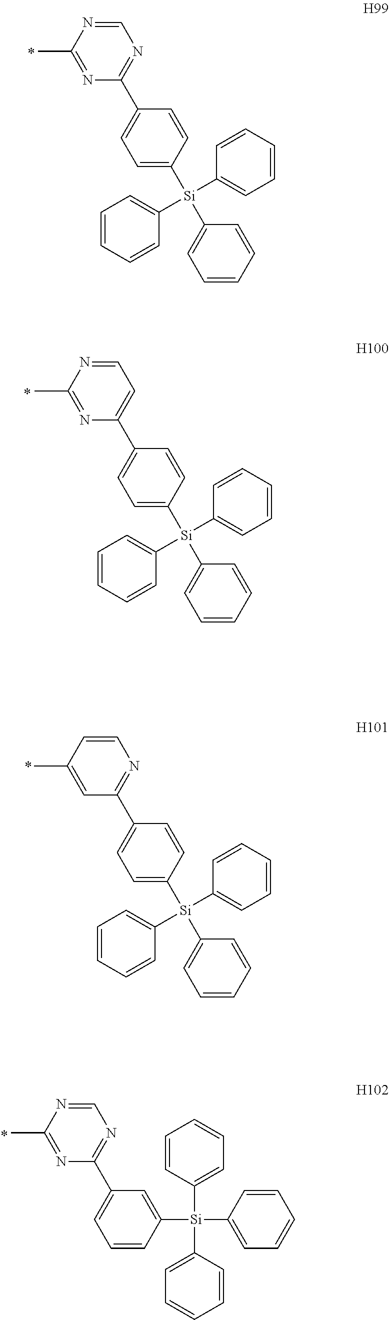

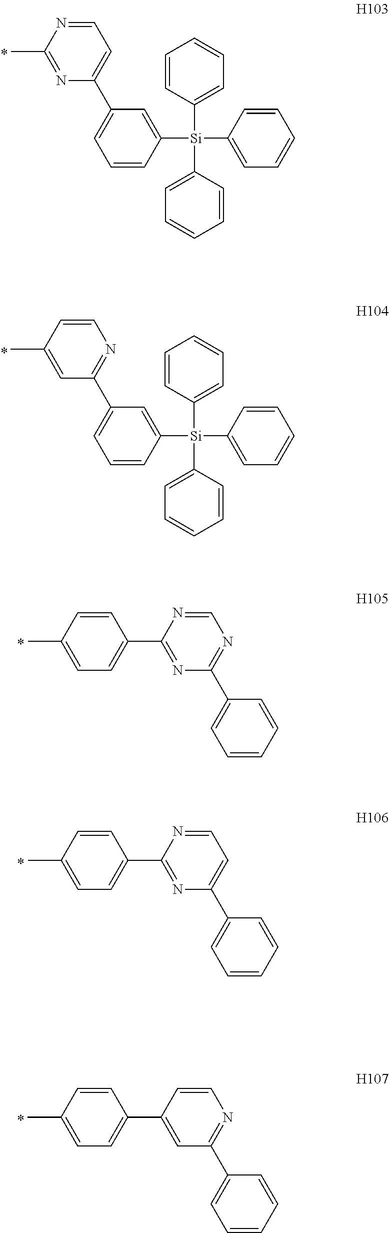

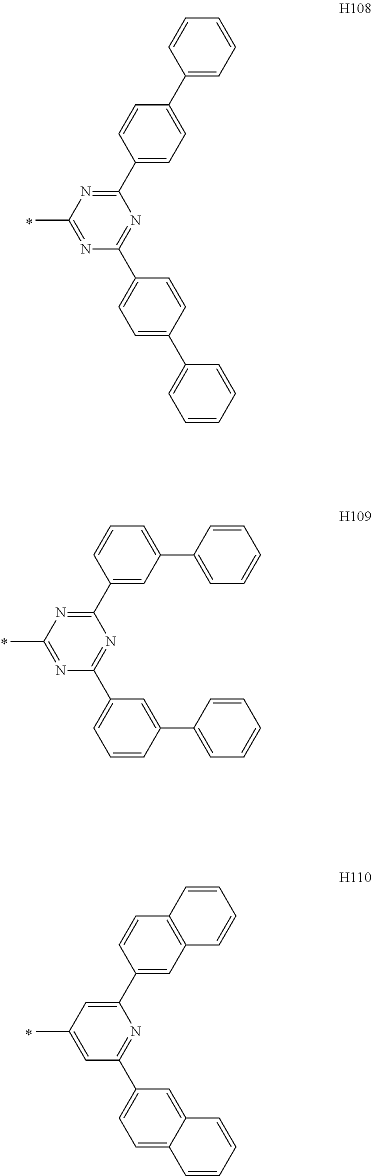

Provided is an organic electroluminescent device according to the present invention, which includes a first electrode; a second electrode; and one or more organic layer(s) interposed between the first electrode and the second electrode, wherein the organic layer includes one or more organic electroluminescent compound(s) by Chemical Formulas 1 to 6.

In the organic electroluminescent device according to the present invention, the organic layer may include an electroluminescent layer, which includes one or more phosphorescence dopants with one or more organic electroluminescent compounds of Chemical Formulas 1 to 6 as an electroluminescent host. The electroluminescent dopant is not specifically limited.

In the organic electroluminescent device of the present invention, one or more organic electroluminescent compounds selected from Chemical Formulas 1 to 6 may be included and one or more compounds selected from the group consisting of arylamine compounds or styrylarylamine compounds may be further included at the same time.

The organic layer may further include one or more metal(s) selected from a group consisting of organic metals of Group 1, Group 2, 4th period and 5th period transition metals, lanthanide metals and d-transition elements or complex compound(s) besides one or more organic electroluminescent compounds selected from Chemical Formulas 1 to 6. The organic layer may include an electroluminescent layer and a charge generating layer.

The organic layer may simultaneously include one or more organic electroluminescent layer(s) emitting blue, red or green light besides the organic electroluminescent compound to form a white light-emitting organic electroluminescent device.

### Advantageous Effects

Since the organic electroluminescent compound according to the present invention exhibits good luminous efficiency and excellent life property compared to the existing host material, it may be used to manufacture OLED devices having very superior operation life and consuming less power due to improved power efficiency.

### MODE OF THE INVENTION

The organic electroluminescent compounds according to the present invention, processes for preparing the same, and luminescence properties of devices employing the same will be described in detail hereinafter based on the representative compound for easy understanding. However, the following examples are provided for illustrative purposes only and they are not intended to limit the scope of the present invention.

### Preparation Example

### Preparation Example 1

### Preparation of Compound A

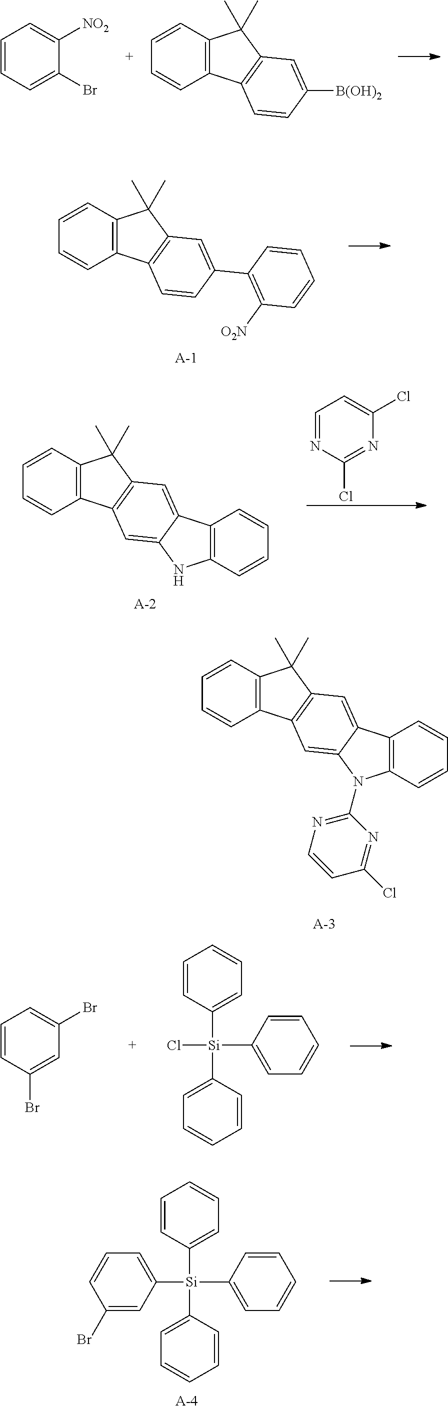

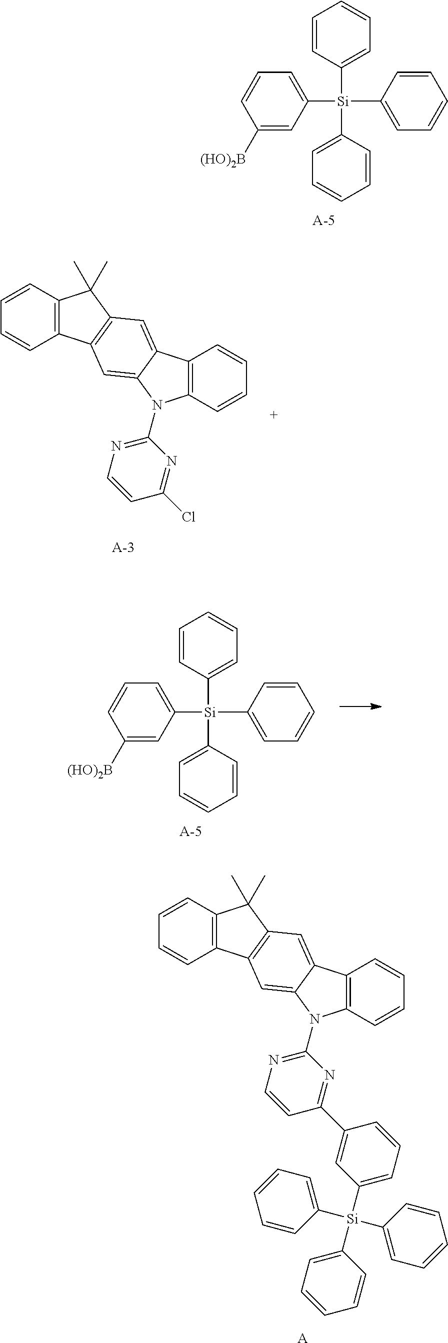

### Preparation of Compound A-1

1-bromonitrobenzene (16 g, 74.25 mmol), 9,9-dimethyl-9H-fluorene-2-ylboronic acid (23 g, 96.60 mmol), Pd(PPh₃)₄ (4.2 g, 3.63 mmol), 2M K₂CO₃ aqueous solution (111 mL), EtOH (100 mL) and toluene (200 mL) were mixed and heated to 120° C. under reflux for 3 hours. After completion of the reaction, the mixture was washed with distilled water. After extracting with EA and drying an organic layer with MgSO₄, solvent was removed by a rotary type evaporator. The residue was purified via column chromatography to obtain Compound A-1 (22 g, 95%).

### Preparation of Compound A-2

Compound A-1 (24 g, 76.10 mmol), triethylphosphite (200 mL) and 1,2-dichlorobenzene (200 mL) were mixed, heated to 180° C. and stirred for 12 hours. When the reaction was completed, unreacted triethylphosphite and 1,2-dichlorobenzene were removed by using a distillation device and the residue was washed with distilled water. After extracting with EA and drying an organic layer with MgSO₄, solvent was removed by a rotary type evaporator. The residue was purified via column chromatography to obtain Compound A-2 (7 g, 33%).

### Preparation of Compound A-3

DMF (10 mL) was added to NaH (60%, 1.15 g, 28.90 mmol) and stirred at room temperature. After dissolving Compound A-2 (6.3 g, 28.98 mmol) in DMF (50 mL), the mixture was slowly added to a reaction vessel containing NaH. After stirring for 1 hour at room temperature, 2,4-dichloropyrimidin (4.9 g, 33.34 mmol) was slowly added thereto and dissolved in DMF 50 mL. After reaction for 5 hours, H₂O (50 mL) was added. After the produced solid was filtered, dissolved in MC, and extracted, an organic layer was dried with MgSO₄. Solvent was removed by a rotary type evaporator. The residue was purified via column chromatography to obtain Compound A-3 (4 g, 45%).

### Preparation of Compound A-4

1,3-dibromobenzene (20 g, 84.77 mmol) was added to a reaction vessel and creates a nitrogen atmosphere in a vacuum state. After adding THF (500 mL), the mixture was stirred at −78° C. for 10 minutes. After slowly adding N-BuLi(2.5M) (33.9 mL, 84.77 mmol), the mixture was stirred at −78° C. for 1 hour. Chlorotriphenylsilane (29.9 g, 107.72 mmol) was dissolved in THF (100 mL) and slowly added thereto. After stirring for 12 hours at room temperature, the reaction was completed and the mixture was washed with distilled water. After extracting with EA and drying an organic layer with MgSO₄, solvent was removed by a rotary type evaporator. Recrystallization from MC and MeOH gave Compound A-4 (62 g, 63%).

### Preparation of Compound A-5

Compound A-4 (22.5 g, 0.10 mol) was added to a reaction vessel and creates a nitrogen atmosphere in a vacuum state. After adding THF (1.3 L), the mixture was stirred at −78° C. for 10 minutes. N-BuLi (2.5M) (48.6 mL, 0.12 mol) was slowly added thereto and stirred at −78° C. for 1 hour. Triethylborate (18 mL, 0.16 mmol) was slowly added. After stirring for 12 hours at room temperature, the reaction was completed and the mixture was washed with distilled water. After extracting with EA and drying an organic layer with MgSO₄, solvent was removed by a rotary type evaporator. The residue was purified via column chromatography to obtain Compound A-5 (10 g, 450).

### Preparation of Compound A

Compound A-3 (2.5 g, 6.31 mmol), Compound A-5 (3.6 g, 9.47 mmol), Pd (PPh₃)₄ (730 mg, 0.63 mmol), K₂CO₃ (2M) (19 mL), EtOH (19 mL) and toluene (40 mL) were mixture and heated to 120° C. under reflux for 3 hours. When the reaction was completed, the mixture was washed with distilled water. After extracting with EA and drying an organic layer with MgSO₄, solvent was removed by a rotary type evaporator. The residue was purified via column chromatography to obtain Compound A (3.8 g, 88%).

### Preparation Example 2

### Preparation of Compound B

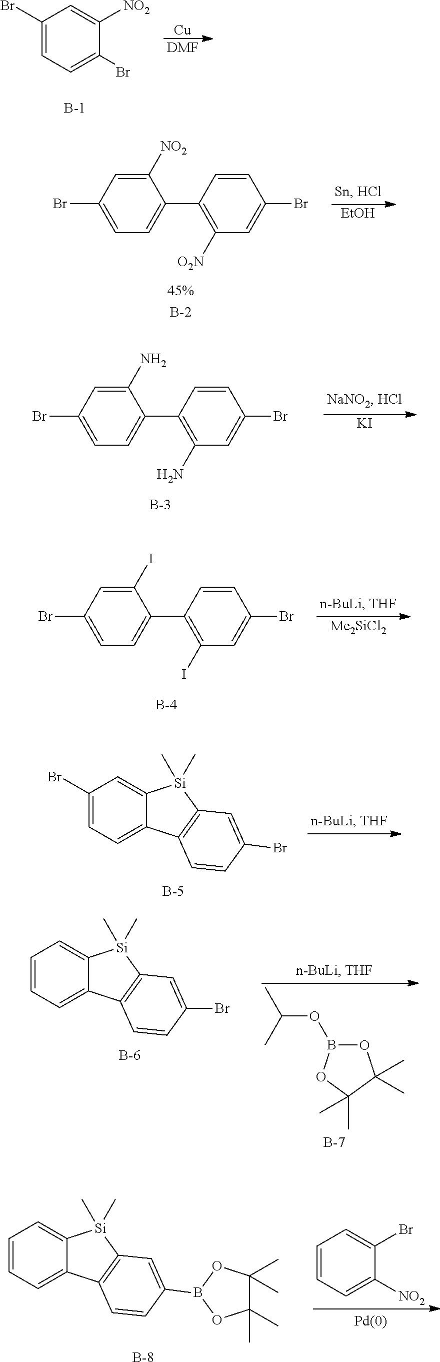

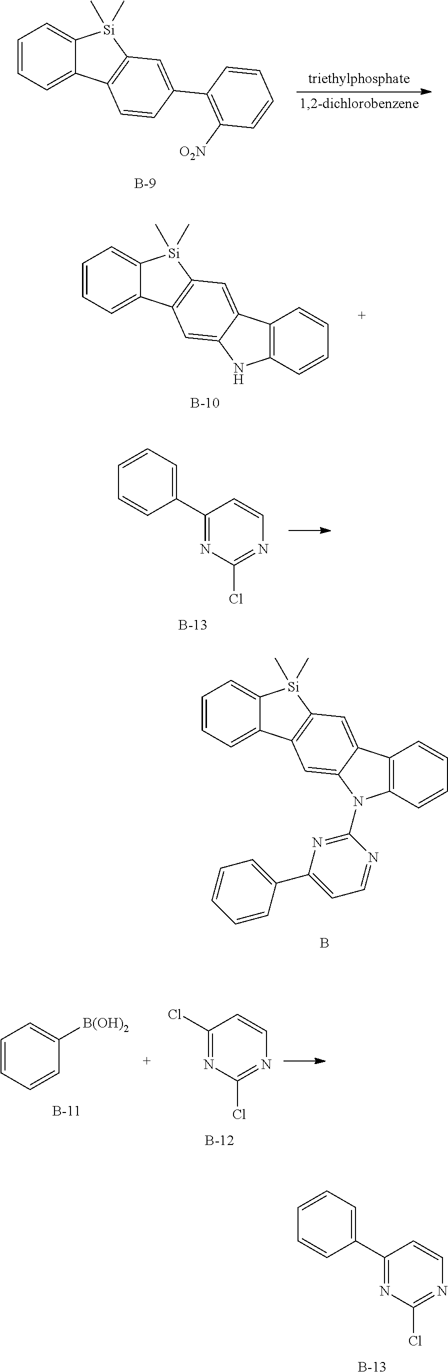

### Preparation of Compound B-2

Compound B-1 (50.0 g, 179 mmol) was dissolved in DMF (200 mL) and copper powder (27.0 g, 424 mmol) was added thereto. The mixture was stirred at 125° C. for 3 hours. The reaction mixture was cooled at room temperature and dried after filtering and removing sediment. Washing with MeOH (500 mL) gave Compound B-2 (27.1 g, 88%).

### Preparation of Compound B-3

Compound B-2 (15 g, 37.3 mmol) was dissolved in ethanol (200 mL) and 32% (w/w) HCl aqueous solution (120 mL) was added thereto. After portion-wise of tin powder (17.6 g, 147 mmol) at room temperature for 10 minutes, it was stirred at 100° C. for 2 hours. After cooling at room temperature, the reaction mixture was added to ice water and became basic by using 20% (w/w) NaOH aqueous solution (150 mL). After extracting with diethylether, washing with bryn and drying, Recrystallization from ethanol gave Compound B-3 (9.2 g, 72%).

### Preparation of Compound B-4

17% (w/w) HCl aqueous solution (85 mL) was added to a round bottom flask containing Compound B-3 (8.5 g, 25 mmol) at 0° C., and NaNO₂ aqueous solution [NaNO₂ 4.3 g (62 mmol)+water (15 mL)] was added thereto. The mixture was stirred for 30 minutes and KI aqueous solution[KI 41.5 g (250 mmol)+water (15 mL)] was added thereto. The mixture was stirred at room temperature for 1 hour and stirred at 60° C. for 3 hours. After neutralizing with saturated KOH solvent, extracting with ethylacetate and washing with saturated Na₂SO₃, the residue was purified via column chromatography to obtain Compound B-4 (4 g, 29%).

### Preparation of Compound B-5

A round bottom flask containing Compound B-4 (4 g, 7.1 mmol) was filled with argon gas and THF 30 mL was added thereto. The mixture was cool to −78° C. n-BuLi (2.5M in hexane, 6.2 mL, 15.6 mmol) was slowly added and stirred for 1 hour. Dichlorodimethylsilane (2.0 g, 15.6 mmol) was added thereto and slowly heated to room temperature under reflux for 12 hours. After extracting with EA and washing with water, the obtained organic layer was dried and purified via silica column chromatography to obtain Compound B-5 (2 g, 76%).

### Preparation of Compound B-6

A round bottom flask containing Compound B-5 (2 g, 5.43 mmol) was filled with argon gas and cooled to −78° C. after adding THF (25 mL) thereto. n-BuLi (2.5M in hexane, 2.2 mL, 5.43 mmol) was slowly added and stirred for 1 hour. 1M HCl (20 mL) was added thereto and stirred for 2 hours. After extracting with EA and washing with water when the mixture was completely stirred, the obtained organic layer was dried and purified via silica column chromatography to obtain Compound B-6 (1.5 g, 960).

### Preparation of Compound B-8

A round bottom flask containing Compound B-6 (15 g, 51.9 mmol) was filled with argon gas and cooled to −78° C. after adding THF (300 mL) thereto. n-BuLi (2.5M in hexane, 20.8 mL, 51.9 mmol) was slowly added and stirred for 1 hour. Compound B-7 (335 mg, 62.3 mmol) was added thereto and slowly heated to room temperature under reflux for 12 hours. After extracting with EA and washing with water, the obtained organic layer was dried and purified via silica column chromatography to obtain Compound B-8 (12 g, 690).

### Preparation of Compound B-9

2-bromonitrobenzene (8.65 g, 42.8 mmol) and Pd(PPh₃)₄ (1.24 g, 1.07 mmol) was added to a round bottom flask containing Compound B-8 (12 g, 35.7 mmol) and the round bottom flask was filled with argon gas. Toluene (120 mL), ethanol (60 mL), and 2M K₂CO₃ (60 mL) were added thereto and stirred under reflux for 4 hours. After cooling at room temperature, extracting with EA and washing with water, the obtained organic layer was dried and purified via silica column chromatography to obtain Compound B-9 (9.5 g, 800).

### Preparation of Compound B-10

A round bottom flask containing Compound B-9 (9.5 g, 28.7 mmol) was filled with argon gas. Triethylphosphite (100 mL) and 1,2-dichlorobenzene (500 mL) were added thereto and stirred under reflux for 12 hours. After cooling at room temperature, extracting with EA and washing with water, the obtained organic layer was dried and purified via silica column chromatography to obtain Compound B-10 (7.2 g, 840).

### Preparation of Compound B-13

Compound B-11 (9.8 g, 80.5 mmol) and Pd (PPh₃)₄ (2.33 g, 2.01 mmol) were added to a round bottom flask containing Compound B-12 (10 g, 67.1 mmol) and the round bottom flask was filled with argon gas. Toluene (240 mL), ethanol (120 mL), and 2M K₂CO₃ (120 mL) were added thereto and stirred under reflux for 4 hours. After cooling at room temperature, extracting with EA and washing with water, the obtained organic layer was dried and purified via silica column chromatography to obtain Compound B-13 (11 g, 860).

### Preparation of Compound B

A mixture that Compound B-10 (3.0 g, 10.0 mmol) was dissolved in DMF (200 mL) was slowly added to a round bottom flask containing NaH (288 mg, 12 mmol) and DMF (100 mL) and stirred for 1 hour. Compound B-13 (1.5 g, 10 mmol) was slowly added to and dissolved in DMF (200 mL) and stirred at room temperature for 12 hours. Filtering the reaction mixture, washing with water and MeOH and drying gave Compound B (2.1 g, 46%).

### Preparation Example 3

### Preparation of Compound C

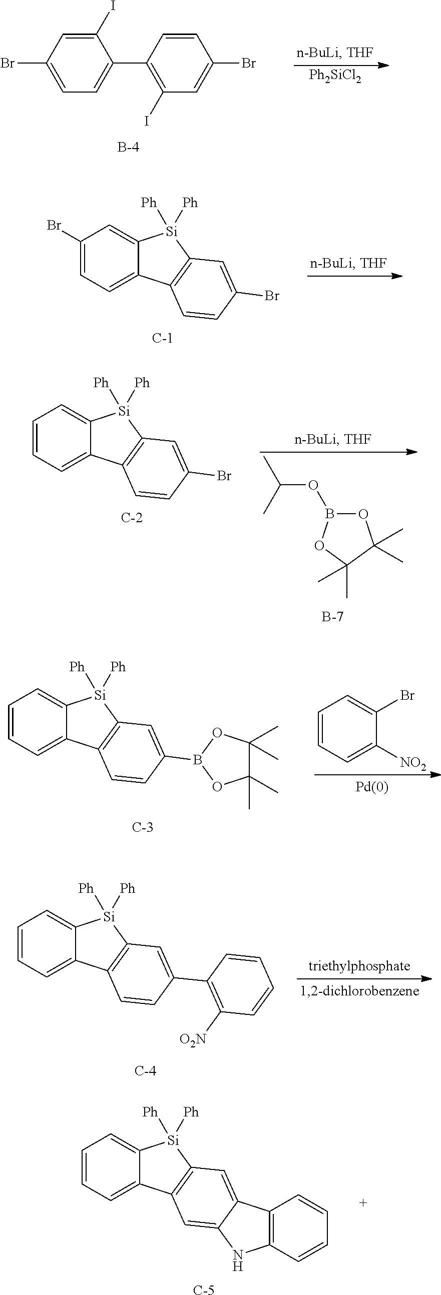

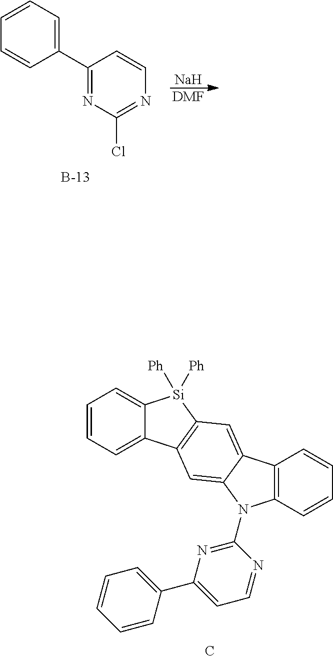

### Preparation of Compound C-1

Except using dichlorodiphenylsilane instead of dichlorodimethylsilane, Compound C-1 (1.7 g, 50%) was prepared using Compound B-4 as starting material in the same manner as preparation of Compound B-5 in Preparation Example 2.

### Preparation of Compound C

Compound C (347 mg, 55%) was prepared using Compound C-1 as starting material in the same manner as preparation of Compounds B-6, B-8, B-9, B-10 and B in Preparation Example 2.

Organic electroluminescent Compounds TA, TB and TC were prepared according to the method of Preparation Examples 1 to 3 and Tables 1 to 4 show ¹H NMR and MS/FAB, which are substituents of the prepared organic electroluminescent compounds.

TABLE 1
TC4

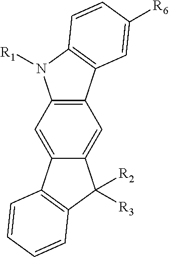

| | | | | | | MS/FAB | |
|---|---|---|---|---|---|---|---|
| Cmpd. | R₁ | R₂ | R₃ | R₆ | ¹H NMR (CDCl₃, 200 MHz) | found | calculated |
| TC4-1 | H1 | methyl | methyl | phenyl | δ = 1.72 (6H, s), 7.05 (2H, m), 7.24 (1H, | 588.74 | 588.26 |
| | | | | | m), 7.41~7.52 (8H, m), 7.54 (5H, s), | | |
| | | | | | 7.61 (1H, m), 7.77 (1H, m), 8 (1H, m), | | |
| | | | | | 8.09 (1H, m), 8.12 (1H, s), 8.18 (1H, | | |
| | | | | | m), 8.3 (4H, m) | | |
| TC4-4 | H4 | methyl | methyl | phenyl | δ = 1.72 (6H, s), 7.24 (1H, m), | 589.73 | 589.25 |
| | | | | | 7.41~7.44 (4H, m), 7.51~7.52 (8H, m), | | |
| | | | | | 7.54 (1H, s), 7.61 (1H, m), | | |
| | | | | | 7.77~7.79 (5H, m), 8 (1H, m), 8.09 (1H, | | |
| | | | | | m), 8.12 (1H, s), 8.18 (1H, m), | | |
| | | | | | 8.63 (1H, s) | | |
| TC4-12 | H12 | methyl | methyl | phenyl | δ = 1.72 (6H, s), 7.24 (1H, m), | 590.71 | 590.25 |
| | | | | | 7.41~7.44 (4H, m), 7.51~7.52 (8H, m), | | |
| | | | | | 7.54 (1H, s), 7.61 (1H, m), 7.77 (1H, | | |
| | | | | | m), 8 (1H, m), 8.09 (1H, m), 8.12 (1H, | | |
| | | | | | s), 8.18 (1H, m), 8.28 (4H, m) | | |
| TC4-19 | H19 | phenyl | phenyl | H | δ = 7.11 (4H, m), 7.24~7.26 (4H, m), | 535.64 | 535.20 |
| | | | | | 7.33 (5H, m), 7.44 (1H, m), 7.5 (1H, s), | | |
| | | | | | 7.61 (1H, m), 7.8 (1H, m), 7.94 (1H, m), | | |
| | | | | | 8.05 (2H, s), 8.05~8.09 (2H, m), | | |
| | | | | | 8.16 (1H, m), 8.55 (1H, m), 9.74 (1H, | | |
| | | | | | m) | | |
| TC4-26 | H26 | phenyl | phenyl | H | δ = 2.2 (1H, m), 6.01 (1H, s), 6.43 (1H, | 675.82 | 675.27 |
| | | | | | s), 7.11~7.17 (8H, m), 7.24~7.26 (4H, | | |
| | | | | | m), 7.33 (7H, m), 7.4~7.44 (5H, m), | | |
| | | | | | 7.5 (1H, s), 7.61 (1H, m), 8.05 (1H, s), | | |
| | | | | | 8.09~8.11 (2H, m), 8.55 (1H, m) | | |
| TC4-32 | H32 | phenyl | phenyl | H | δ = 7.11 (4H, m), 7.24~7.26 (4H, m), | 540.68 | 540.17 |
| | | | | | 7.33 (5H, m), 7.44 (1H, m), 7.5 (1H, s), | | |
| | | | | | 7.53 (2H, m), 7.61 (1H, m), 7.94 (1H, | | |
| | | | | | m), 8.01 (1H, m), 8.05 (1H, s), | | |
| | | | | | 8.09 (1H, m), 8.18 (1H, m), 8.55 (1H, | | |
| | | | | | m) | | |
| TC4-36 | H36 | phenyl | phenyl | phenyl | δ = 7.11 (4H, m), 7.24~7.26 (3H, m), | 627.73 | 627.23 |
| | | | | | 7.33 (4H, m), 7.41~7.44 (3H, m), | | |
| | | | | | 7.5 (1H, s), 7.51~7.52 (6H, m), | | |
| | | | | | 7.61 (1H, m), 7.77 (1H, m), 8 (1H, m), | | |
| | | | | | 8.05 (3H,s), 8.05~8.09 (1H, m), | | |
| | | | | | 8.18 (1H, m) | | |
| TC4-41 | H41 | methyl | methyl | phenyl | δ = 1.72 (2H, s), 7.24 (1H, m), | 578.70 | 578.25 |
| | | | | | 7.41~7.52 (12H, m), 7.54 (1H, s), | | |
| | | | | | 1.58~7.61 (3H, m), 7.77 (1H, m), 8 (1H, | | |
| | | | | | m), 8.09 (1H, m), 8.12 (1H, s), | | |
| | | | | | 8.18 (1H, m), 8.28 (2H, m) | | |
| TC4-42 | H42 | methyl | methyl | phenyl | δ = 1.72 (2H, s), 7.24~7.33 (4H, m), | 600.75 | 600.26 |
| | | | | | 7.41~7.52 (7H, m), 7.54 (1H, s), | | |
| | | | | | 7.61~7.63 (6H, m), 7.77 (1H, m), | | |
| | | | | | 7.94~8 (2H, m), 8.09 (1H, m), 8.12 (2H, | | |
| | | | | | s), 8.18 (1H, m), 8.55 (1H, m) | | |
| TC4-44 | H44 | methyl | methyl | phenyl | δ = 1.72 (2H, s), 7.24~7.25 (2H, m), | 601.74 | 601.25 |
| | | | | | 7.33 (1H, m), 7.41~7.44 (2H, m), | | |
| | | | | | 7.51~7.52 (5H, m), 7.54 (1H, s), | | |
| | | | | | 7.61~7.62 (5H, m), 7.77 (1H, m), | | |
| | | | | | 7.94~8 (2H, m), 8.09 (1H, m), 8.12 (1H, | | |
| | | | | | s), 8.18 (1H, m), 8.43 (1H, m), | | |
| | | | | | 8.55 (1H, m), 9.34 (1H, m) | | |
| TC4-48 | H48 | methyl | methyl | phenyl | δ = 1.72 (6H, s), 7.24 (1H, m), | 602.73 | 602.25 |
| | | | | | 7.41~7.44 (2H, m), 7.51~7.52 (6H, m), | | |
| | | | | | 7.54 (1H, s), 7.61~7.62 (5H, m), | | |
| | | | | | 7.77 (1H, m), 8 (1H, m), 8.09 (1H, m), | | |
| | | | | | 8.12 (1H, s), 8.18 (1H, m), 8.43 (2H, | | |
| | | | | | m), 9.34 (2H, m) | | |
| TC4-49 | H49 | phenyl | phenyl | H | δ = 1.72 (6H, s), 6.55 (2H, m), 6.63 (2H, | 690.87 | 690.30 |
| | | | | | m), 6.73 (2H, m), 7.02~7.05 (4H, m), | | |
| | | | | | 7.11 (4H, m), 7.24~7.26 (4H, m), | | |
| | | | | | 7.33~7.37 (7H, m), 7.44 (1H, m), | | |
| | | | | | 7 .5 (1H, s), 7.61 (1H, m), 7.94 (1H, m), | | |
| | | | | | 8.05 (1H, s), 8.09 (1H, m), 8.55 (1H, | | |
| | | | | | m) | | |
| TC4-54 | H54 | phenyl | phenyl | H | δ = 2.88 (4H, m), 6.58~6.63 (4H, m), | 676.84 | 676.29 |
| | | | | | 6.76 (2H, m), 7.02~7.04 (4H, m), | | |
| | | | | | 7.11 (4H, m), 7.24~7.26 (4H, m), | | |
| | | | | | 7.33~7.37 (7H, m), 7.44 (1H, m), | | |
| | | | | | 7.5 (1H, s), 7.61 (1H, m), 7.94 (1H, m), | | |
| | | | | | 8.05 (1H, s), 8.09 (1H, m), 8.55 (1H, m) | | |
| TC4-58 | H58 | methyl | methyl | phenyl | δ = 1.72 (6H, s), 6.52 (1H, m), 6.87 (1H, | 550.69 | 550.24 |
| | | | | | m), 7.24 (1H, m), 7.33 (1H, m), | | |
| | | | | | 7.41~7.44 (2H, m), 7.51~7.52 (4H, m), | | |
| | | | | | 7.54 (1H, s), 7.6~7.62 (6H, m), | | |
| | | | | | 7.77 (1H, m), 7.93~8 (3H, m), 8.09 (1H, | | |
| | | | | | m), 8.12 (1H, s), 8.18 (1H, m) | | |
| TC4-64 | H64 | methyl | methyl | phenyl | δ = 1.72 (6H, s), 6.63 (2H, m), 6.81(1H, | 632.81 | 632.23 |
| | | | | | m), 6.97~7 (2H, m), 7.07 (1H, m), | | |
| | | | | | 7.16~7.24 (7H, m), 7.41~7.44 (2H, m), | | |
| | | | | | 7.51~7.52 (4H, m), 7.54 (1H, s), | | |
| | | | | | 7.61 (1H, m), 7.77 (1H, m), 8 (1H, m), | | |
| | | | | | 8.09 (1H, m), 8.12 (1H, s), 8.18 (1H, | | |
| | | | | | m) | | |
| TC4-70 | H70 | phenyl | phenyl | H | δ = 7.11 (4H, m), 7.24~7.26 (4H, m), | 561.67 | 561.22 |
| | | | | | 7.32~7.33 (6H, m), 7.44 (1H, m), | | |
| | | | | | 7.5 (1H, s), 7.61 (1H, m), 7.72 (1H, m), | | |
| | | | | | 7.86 (1H, m), 7.94 (1H, m), 8.05 (1H, | | |
| | | | | | s), 8.09 (1H, m), 8.5~8.55 (3H, m), | | |
| | | | | | 8.78 (2H, m) | | |
| TC4-79 | H79 | methyl | methyl | phenyl | δ = 1.72 (6H, s), 7.24 (1H, m), | 591.70 | 591.24 |
| | | | | | 7.41~7.44 (2H, m), 7.51~7.52 (4H, m), | | |
| | | | | | 7.54 (1H, s), 7.61 (1H, m), 7.77 (1H, | | |
| | | | | | m), 7.99~8 (5H, m), 8.09 (1H, m), | | |
| | | | | | 8.12 (1H, s), 8.18 (1H, m), 8.75 (5H, | | |
| | | | | | m) | | |
| TC4-83 | H83 | methyl | methyl | H | δ = 1.72 (6H, s), 7.24~7.25 (2H, m), | 713.80 | 713.26 |
| | | | | | 7.33 (1H, m), 7.41~7.51 (10H, m), | | |
| | | | | | 7.54 (1H, s), 7.61 (1H, m), | | |
| | | | | | 7.77~7.83 (10H, m), 7.94 (1H, m), | | |
| | | | | | 8.09 (1H, m), 8.12 (1H, s), 8.55 (1H, | | |
| | | | | | m), 8.63 (1H, s) | | |
| TC4-86 | H86 | methyl | methyl | H | δ = 1.72 (6H, s), 2.59 (6H, s), | 628.80 | 628.29 |
| | | | | | 7.24~7.33 (6H, m), 7.44~7.5 (4H, m), | | |
| | | | | | 7.54 (1H, s), 7.61~7.67 (6H, m), | | |
| | | | | | 7.94 (2H, m), 8.09 (1H, m), 8.12 (2H, | | |
| | | | | | s), 8.55 (2H, m) | | |
| TC4-89 | H89 | methyl | methyl | phenyl | δ = 1.72 (6H, s), 1.88~1.91 (4H, m), | 670.84 | 670.31 |
| | | | | | 2.3~2.33 (2H, m), 3.22 (1H, m), | | |
| | | | | | 3.7 (1H, m), 7.24 (1H, m), | | |
| | | | | | 7.39~7.44 (5H, m), 7.51~7.52 (8H, m), | | |
| | | | | | 7.54 (1H, s), 7.61 (1H, m), 7.77 (1H, | | |
| | | | | | m), 8.09 (1H, m), 8.12 (1H, s), | | |
| | | | | | 8.27~8.28 (5H, m) | | |
| TC4-91 | H91 | methyl | methyl | phenyl | δ = 1.72 (6H, s), 5.6 (1H, m), 6.9 (1H, | 616.75 | 616.26 |
| | | | | | m), 7.24 (1H, m), 7.41~7.44 (4H, m), | | |
| | | | | | 7.51~7.52 (8H, m), 7.54 (1H, s), | | |
| | | | | | 7.61 (1H, m), 8 (1H, m), 8.09 (1H, m), | | |
| | | | | | 8.12 (1H, s), 8.18~8.2 (2H, m), | | |
| | | | | | 8.28 (4H, m) | | |
| TC4-93 | H93 | methyl | methyl | phenyl | δ = 1.72 (6H, s), 5.11 (2H, s), 7.24 (1H, | 604.74 | 604.26 |
| | | | | | m), 7.39~7.44 (5H, m), 7.51~7.52 (8H, | | |
| | | | | | m), 7.54 (1H, s), 7.61 (1H, m), | | |
| | | | | | 7.77 (1H, m), 8.09 (1H, m), 8.12 (1H, | | |
| | | | | | s), 8.27~8.28 (5H, m) | | |
| TC4-96 | H96 | methyl | methyl | H | δ = 1.72 (6H, s), 7.24~7.25 (2H, m), | 590.71 | 590.25 |
| | | | | | 7.33 (1H, m), 7.41~7.44 (3H, m), | | |
| | | | | | 7.51 (4H, m), 7.54 (1H, s), 7.61 (1H, | | |
| | | | | | m), 7.68 (2H, m), 7.79 (2H, m), | | |
| | | | | | 7.94 (1H, m), 8.09 (1H, m), 8.12 (1H, | | |
| | | | | | s), 8.28 (4H, m), 8.55 (1H, m) | | |
| TC4-97 | H97 | methyl | methyl | phenyl | δ = 1.72 (6H, s), 7.24 (1H, m), | 665.82 | 665.28 |
| | | | | | 7.41~7.44 (4H, m), 7.51~7.52 (8H, m), | | |
| | | | | | 7.54 (1H, s), 7.61 (1H, m), 7.68 (2H, | | |
| | | | | | m), 7.77~7.79 (7H, m), 8 (1H, m), | | |
| | | | | | 8.09 (1H, m), 8.12 (1H, s), 8.18 (1H, | | |
| | | | | | m), 8.23 (1H, s) | | |
| TC4-98 | H98 | methyl | methyl | H | δ = 1.72 (6H, s), 7.24~7.25 (4H, m), | 666.81 | 666.28 |
| | | | | | 7.33 (1H, m), 7.41~7.44 (3H, m), | | |
| | | | | | 7.51 (4H, m), 7.54 (1H, s), 7.61(1H, | | |
| | | | | | m), 7.68 (2H, m), 7.79~7.85 (4H, m), | | |
| | | | | | 7.94 (1H, m), 8.09 (1H, m), 8.12 (1H, | | |
| | | | | | s), 8.28 (4H, m), 8.55 (1H, m) | | |
| TC4-99 | H99 | methyl | methyl | H | δ = 1.72 (6H, s), 7.24~7.29 (2H, m), | 696.91 | 696.27 |
| | | | | | 7.37 (6H, m), 7.44~7.52 (10H, m), | | |
| | | | | | 7.54(1H, s), 7.55 (3H, m), | | |
| | | | | | 7.61~7.63 (2H, m), 7.89 (2H, m), | | |
| | | | | | 8.09~8.12 (2H, m), 8.59 (1H, s), | | |
| | | | | | 8.85 (1H, s) | | |
| TC4-100 | H100 | methyl | methyl | H | δ = 1.72 (6H, s), 7.24~7.29 (2H, | 695.92 | 695.28 |
| | | | | | 7.37 (6H, m), 7.44~7.52 (10H, m), | | |
| | | | | | 7.54 (1H, s), 7.55 (3H, m), | | |
| | | | | | 7.61~7.63 (2H, m), 7.89 (2H, m), | | |
| | | | | | 7.96 (1H, m), 8.09~8.12 (2H, m), | | |
| | | | | | 8.57 (1H, m), 8.85 (1H, s) | | |
| TC4-101 | H101 | methyl | methyl | H | δ = 1.72 (6H, s), 7.24~7.31 (3H, m), | 694.94 | 694.28 |
| | | | | | 7.37 (6H, m), 7.44~7.5 (9H, m), | | |
| | | | | | 7.54 (1H, s), 7.55 (5H, m), | | |
| | | | | | 7.61~7.63 (2H, m), 8.09~8.12 (2H, m), | | |
| | | | | | 8.38~8.4 (3H, m), 8.85 (1H, s) | | |
| TC4-102 | H102 | methyl | methyl | H | δ = 1.72 (6H, s), 7.24~7.29 (2H, m), | 696.91 | 696.27 |
| | | | | | 7.37~7.5 (15H, m), 7.54 (1H, s), | | |
| | | | | | 7.55 (3H, m), 7.61~7.63 (3H, m), | | |
| | | | | | 7.76 (1H, m), 8.09~8.12 (2H, m), | | |
| | | | | | 8.38 (1H, m), 8.59 (1H, s), 8.85 (1H, | | |
| | | | | | s) | | |
| TC4-103 | H103 | methyl | methyl | H | δ = 1.72 (6H, s), 7.24~7.29 (2H, m), | 695.92 | 695.28 |
| | | | | | 7.37~7.5 (15H, m), 7.54 (1H, s), | | |
| | | | | | 7.55 (3H, m), 7.61~7.63 (3H, m), | | |
| | | | | | 7.76 (1H, m), 7.89 (1H, m), 7.96 (1H, | | |
| | | | | | m), 8.09~8.12 (2H, m), 8.57 (1H, m), | | |
| | | | | | 8.85 (1H, s) | | |
| TC4-104 | H104 | methyl | methyl | H | δ = 1.72 (6H, s), 7.24~7.31 (3H, m), | 694.94 | 694.28 |
| | | | | | 7.37 (6H, m), 7.44~7.5 (10H, m), | | |
| | | | | | 7.54 (1H, s), 7.55 (3H, m), | | |
| | | | | | 7.61~7.64 (3H, m), 8.09~8.12 (2H, m), | | |
| | | | | | 8.27 (1H, m), 8.38~8.4 (2H, m), | | |
| | | | | | 8.85 (1H, s) | | |
| TC4-105 | H105 | methyl | methyl | H | δ = 1.72 (6H, s), 7.24~7.29 (2H, m), | 514.62 | 514.22 |
| | | | | | 7.41~7.51 (5H, m), 7.54 (1H, s), | | |
| | | | | | 7.61~7.68 (4H, m), 7.79 (2H, m), | | |
| | | | | | 8.09~8.12 (2H, m), 8.28 (2H, m), | | |
| | | | | | 8.31 (1H, s), 8.85 (1H, s) | | |
| TC4-106 | H106 | methyl | methyl | H | δ = 1.72 (6H, s), 7.24~7.29 (2H, m), | 513.63 | 513.22 |
| | | | | | 7.41~7.51 (5H, m), 7.54 (1H, s), | | |
| | | | | | 7.56~7.68 (5H, m), 7.79 (4H, m), | | |
| | | | | | 8.09~8.12 (2H, m), 8.54 (1H, m), | | |
| | | | | | 8.85 (1H, s) | | |
| TC4-107 | H107 | methyl | methyl | H | δ = 1.72 (6H, s), 7.24~7.29 (2H, m), | 512.64 | 512.23 |
| | | | | | 7.44~7.5 (3H, m), 7.54 (3H, s), | | |
| | | | | | 7.61~7.68 (4H, m), 7.79 (2H, m), 8 (1H, | | |
| | | | | | m), 8.09~8.12 (2H, m), 8.3 (2H, m), | | |
| | | | | | 8.44 (1H, m), 8.6 (1H, m), 8.85 (1H, s) | | |
| TC4-108 | H108 | methyl | methyl | H | δ = 1.72 (6H, s), 7.24~7.29 (6H, | 666.81 | 666.28 |
| | | | | | 7.41~7.52 (12H, m), 7.54 (1H, s), | | |
| | | | | | 7.61~7.63 (2H, m), 7.85 (4H, m), | | |
| | | | | | 8.09~8.12 (2H, m), 8.85 (1H, s) | | |
| TC4-109 | H109 | methyl | methyl | H | δ = 1.72 (6H, s), 7.24~7.29 (2H, m), | 665.82 | 665.28 |
| | | | | | 7.41~7.52 (14H, m), 7.54 (1H, s), | | |
| | | | | | 7.57~7.63 (4H, m), 7.7~7.75 (4H, m), | | |
| | | | | | 8.09~8.12 (2H, m), 8.63 (1H, s), | | |
| | | | | | 8.85 (1H, s) | | |
| TC4-110 | H110 | methyl | methyl | H | δ = 1.72 (6H, s), 7.05 (2H, m), | 612.76 | 612.26 |
| | | | | | 7.24~7.29 (2H, m), 7.44~7.5 (2H, m), | | |
| | | | | | 7.54 (1H, s), 7.59~7.63 (6H, m), | | |
| | | | | | 7.95~8 (6H, m), 8.09~8.12 (2H, m), | | |
| | | | | | 8.38 (2H, m), 8.85 (3H, s) | | |

TABLE 2
TC10

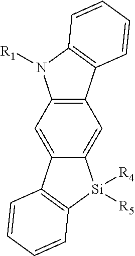

| | | | | | MS/FAB | |
|---|---|---|---|---|---|---|
| Cmpd. | R₁ | R₄ | R₅ | ¹H NMR (CDCl₃, 200 MHz) | found | calculated |
| TC10-3 | H3 | phenyl | phenyl | δ = 7.11 (1H, m), 7.25 (1H, m), 7.33 (2H, | 652.86 | 652.23 |
| | | | | m), 7.37~7.47 (19H, m), 7.61 (1H, m), | | |
| | | | | 7.72 (1H, s), 7.89 (2H, s), | | |
| | | | | 7.89~7.94 (1H, m), 8.3 (2H, m), | | |
| | | | | 8.55~8.6 (2H, m) | | |
| TC10-5 | H5 | phenyl | phenyl | δ = 7.14 (2H, m), 7.25 (1H, m), | 654.83 | 654.22 |
| | | | | 7.33~7.37 (6H, m), 7.46~7.55 (7H, m), | | |
| | | | | 7.61 (1H, m), 7.7 (2H, m), 7.72 (1H, s), | | |
| | | | | 7.89 (2H, s), 7.89~7.94 (1H, m), | | |
| | | | | 8.15 (2H, m), 8.53~8.55 (3H, m), | | |
| | | | | 9.3 (2H, m) | | |
| TC10-6 | H6 | phenyl | phenyl | δ = 7.25 (1H, m), 7.32 (1H, s), | 653.84 | 653.23 |
| | | | | 7.33~7.55 (19H, m), 7.61 (1H, m), | | |
| | | | | 7.72 (1H, s), 7.79 (2H, m), 7.89 (2H, s), | | |
| | | | | 7.89~7.94 (1H, m), 8.28 (2H, m), | | |
| | | | | 8.55 (1H, m) | | |
| TC10-11 | H11 | phenyl | phenyl | δ = 7.25 (1H, m), 7.33~7.55 (16H, m), | 577.75 | 577.20 |
| | | | | 7.61 (1H, m), 7.72 (1H, s), 7.84 (1H, m), | | |
| | | | | 7.89 (2H, s), 7.89~7.94 (1H, m), | | |
| | | | | 8.05 (2H, m), 8.55 (1H, m), 8.98 (1H, m) | | |
| TC10-14 | H14 | phenyl | phenyl | δ = 7.25 (1H, m), 7.33 (2H, m), | 626.82 | 626.22 |
| | | | | 7.37~7.46 (18H, m), 7.61(1H, m), | | |
| | | | | 7.72 (1H, s), 7.76 (1H, m), 7.89 (2H, | | |
| | | | | 7.89~7.94 (2H, m), 8.43 (1H, s), | | |
| | | | | 8.55 (1H, m) | | |
| TC10-28 | H28 | phenyl | phenyl | δ = 6.47 (1H, m), 6.58 (1H, m), 7.25 (1H, | 640.85 | 640.23 |
| | | | | m), 7.33 (2H, m), 7.37 (4H, m), 7.41 (1H, | | |
| | | | | m), 7.45~7.51 (15H, m), 7.72 (1H, s), | | |
| | | | | 7.79 (2H, m), 7.89 (2H, s), | | |
| | | | | 7.89~7.94 (1H, m), 8.55 (1H, m) | | |
| TC10-33 | H33 | phenyl | phenyl | δ = 7.25 (1H, m), 7.33~7.55 (16H, m), | 583.78 | 583.15 |
| | | | | 7.61 (1H, m), 7.72 (1H, s), 7.89 (2H, s), | | |
| | | | | 7.89~7.94 (1H, m), 8.03 (2H, m), | | |
| | | | | 8.55 (1H, m) | | |
| TC10-40 | H40 | phenyl | phenyl | δ = 7.25 (1H, m), 7.33~7.37 (6H, | 641.83 | 641.23 |
| | | | | 7.4 (1H, s), 7.41 (1H, m), | | |
| | | | | 7.45~7.51 (15H, m), 7.72 (1H, s), | | |
| | | | | 7.89 (2H, s), 7.89~7.94 (1H, m), | | |
| | | | | 8.28 (2H, m), 8.55 (1H, m) | | |
| TC10-52 | H52 | phenyl | phenyl | δ = 6.63 (2H, m), 6.97 (2H, m), | 696.93 | 696.21 |
| | | | | 7.16~7.25 (7H, m), 7.33~7.37 (8H, | | |
| | | | | 7.46~7.55 (7H, m), 7.61 (1H, m), | | |
| | | | | 7.72 (1H, s), 7.89 (2H, s), | | |
| | | | | 7.89~7.94 (1H, m), 8.55 (1H, m) | | |
| TC10-60 | H60 | phenyl | phenyl | δ = 3.3 (1H, m), 3.9 (1H, s), 5.78 (1H, | 693.91 | 693.26 |
| | | | | m), 6.07 (1H, m), 6.41 (1H, m), 6.79 (1H, | | |
| | | | | m), 6.94 (2H, m), 7.23~7.27 (7H, m), | | |
| | | | | 7.33~7.37 (6H, m), 7.46~7.55 (7H, m), | | |
| | | | | 7.61 (1H, m), 1.12 (1H, s), 7.86 (1H, m), | | |
| | | | | 7.89 (2H, s), 7.89~7.94 (1H, m), | | |
| | | | | 8.55 (1H, m) | | |
| TC10-63 | H63 | phenyl | phenyl | δ = 0.66 (6H, s), 6.63 (2H, m), | 723.02 | 722.26 |
| | | | | 6.73~6.74 (3H, m), 6.81 (1H, m), | | |
| | | | | 7.08 (1H, m), 7.2~7.37 (12H, m), | | |
| | | | | 7.46~7.55 (7H, m), 7.61 (1H, m), | | |
| | | | | 7.72 (1H, s), 7.89 (2H, s), | | |
| | | | | 7.89~7.94 (1H, m), 8.55 (1H, m) | | |
| TC10-72 | H72 | phenyl | phenyl | δ = 7.25 (1H, m), 7.33~7.37 (6H, | 577.75 | 577.20 |
| | | | | 7.46~7.61 (10H, m), 7.72 (1H, s), | | |
| | | | | 7.89 (2H, s), 7.89~7.94 (1H, m), | | |
| | | | | 8.4~8.47 (3H, m), 8.55 (1H, m), 8.7 | | |
| | | | | m), 9.24 (1H, m) | | |
| TC10-77 | H77 | phenyl | phenyl | δ = 7.25 (1H, m), 7.33~7.37 (6H, m), | 576.76 | 576.20 |
| | | | | 7.46~7.61 (9H, m), 7.68 (2H, m), | | |
| | | | | 7.72 (1H, s), 7.79 (2H, m), 7.89 (2H, s), | | |
| | | | | 7.89~7.94 (1H, m), 8.42 (1H, m), | | |
| | | | | 8.55 (1H, m), 8.7 (1H, m), 9.24 (1H, m) | | |
| TC10-80 | H80 | phenyl | phenyl | δ = 6.95~7.01 (3H, m), 7.25 (1H, m), | 669.84 | 669.22 |
| | | | | 7.28~7.41 (18H, m), 7.56 (1H, s), | | |
| | | | | 7.61 (1H, m), 7.72 (1H, s), 7.79 (2H, m), | | |
| | | | | 7.89 (2H, s), 7.89~7.94 (1H, m), | | |
| | | | | 8.55 (111, m) | | |
| TC10-82 | H82 | phenyl | phenyl | δ = 1.73 (4H, m), 1.88 (4H, m), 2.72 (1H, | 736.98 | 736.30 |
| | | | | m), 3.64 (1H, m), 7.33 (1H, m), 7.37 (4H, | | |
| | | | | m), 7.41~7.51 (17H, m), 7.72 (1H, s), | | |
| | | | | 7.89 (2H, s), 8.17 (1H, m), 8.28 (4H, m) | | |
| TC10-88 | H88 | phenyl | phenyl | δ = 1.94 (2H, m), 2.96 (2H, m), 3.7 (1H, | 633.85 | 633.26 |
| | | | | m), 7.1 (1H, m), 7.33~7.46 (18H, m), | | |
| | | | | 7.72 (1H, s), 7.86 (1H, m), 7.89 (2H, s), | | |
| | | | | 7.89~7.9 (1H, m), 8.17 (1H, m), | | |
| | | | | 8.48 (1H, m) | | |
| TC10-92 | H92 | phenyl | phenyl | δ = 7.25 (1H, m), 7.33~7.55 (19H, m), | 678.85 | 678.22 |
| | | | | 7.61 (1H, m), 7.72 (1H, s), 7.89 (2H, s), | | |
| | | | | 7.89~7.94 (1H, m), 8.28 (4H, m), | | |
| | | | | 8.55 (1H, m) | | |
| TC10-94 | H94 | phenyl | phenyl | δ = 7.25 (1H, m), 7.33 (2H, m), 7.37 (4H, | 717.93 | 717.26 |
| | | | | m), 7.41 (2H, m), 7.45 (1H, m), | | |
| | | | | 7.46~7.51 (18H, m), 7.72 (1H, s), | | |
| | | | | 7.79 (2H, m), 7.89 (2H, s), | | |
| | | | | 7.89~7.94 (1H, m), 8.55 (1H, m) | | |
| TC10-99 | H99 | phenyl | phenyl | δ = 7.29~7.37 (12H, m), 7.46~7.55 (19H, | 837.13 | 836.28 |
| | | | | m), 7.61~7.63 (2H, m), 7.72 (1H, s), | | |
| | | | | 7.89 (4H, s), 8.12 (1H, m), 8.59 (1H, s) | | |
| TC10-100 | H100 | phenyl | phenyl | δ = 7.29~7.37 (12H, m), 7.46~7.55 (19H, | 836.14 | 835.28 |
| | | | | m), 7.61~7.63 (2H, m), 7.72 (1H, s), | | |
| | | | | 7.89 (4H, s), 7.96 (1H, m), 8.12 (1H, m), | | |
| | | | | 8.57 (1H, m) | | |
| TC10-101 | H101 | phenyl | phenyl | δ = 7.29~7.37 (13H, m), 7.45~7.55 (20H, | 835.15 | 834.29 |
| | | | | m), 7.61~7.63 (2H, m), 7.72 (1H, s), | | |
| | | | | 7.89 (2H, s), 8.12 (1H, m), | | |
| | | | | 8.38~8.4 (3H, m) | | |
| TC10-102 | H102 | phenyl | phenyl | δ = 7.29~7.42 (30H, m), 7.61~7.63 (3H, | 837.13 | 836.28 |
| | | | | m), 7.72 (1H, s), 7.76 (1H, m), 7.89 (2H, | | |
| | | | | s), 8.12 (1H, m), 8.38 (1H, m), 8.59 (1H, | | |
| | | | | s) | | |
| TC10-103 | H103 | phenyl | phenyl | δ = 7.29~7.42 (30H, m), 7.61~7.63 (3H, | 836.14 | 835.28 |
| | | | | m), 7.72 (1H, s), 7.76 (1H, m), 7.89 (3H, | | |
| | | | | s), 7.96 (1H, m), 8.12 (1H, m), 8.57 (1H, | | |
| | | | | m) | | |
| TC10-104 | H104 | phenyl | phenyl | δ = 7.29~7.37 (13H, m), 7.45~7.55 (19H, | 835.15 | 834.29 |
| | | | | m), 7.61~7.64 (3H, m), 7.72 (1H, s), | | |
| | | | | 7.89 (2H, s), 8.12 (1H, m), 8.27 (1H, m), | | |
| | | | | 8.38~8.4 (2H, m) | | |
| TC10-105 | H105 | phenyl | phenyl | δ = 7.29 (1H, m), 7.33~7.46 (16H, m), | 654.83 | 654.22 |
| | | | | 7.61~7.68 (4H, m), 7.72 (1H, s), | | |
| | | | | 7.79 (2H, m), 7.89 (2H, s), 8.12 (1H, m), | | |
| | | | | 8.28 (2H, m), 8.31 (1H, s) | | |
| TC10-106 | H106 | phenyl | phenyl | δ = 7.29 (1H, m), 7.33 (1H, m), 7.37 (4H, | 653.84 | 653.23 |
| | | | | m), 7.41(1H, m), 7.46 (4H, m), | | |
| | | | | 7.5~7.55 (11H, m), 7.72 (1H, s), | | |
| | | | | 7.79 (4H, m), 7.89 (2H, s), 8.12 (1H, m), | | |
| | | | | 8.54 (1H, m) | | |
| TC10-107 | H107 | phenyl | phenyl | δ = 7.29~7.37 (6H, m), 7.46~7.55 (11H, | 652.86 | 652.23 |
| | | | | m), 7.61~7.68 (4H, m), 7.72 (1H, s), | | |
| | | | | 7.79 (2H, m), 7.89 (2H, s), 8 (1H, m), | | |
| | | | | 8.12 (1H, m), 8.3 (2H, m), 8.44 (1H, m), | | |
| | | | | 8.6 (1H, m) | | |
| TC10-108 | H108 | phenyl | phenyl | δ = 7.25 (4H, m), 7.29 (1H, m), | 807.02 | 806.29 |
| | | | | 7.33~7.46 (23H, m), 7.61~7.63 (2H, m), | | |
| | | | | 7.72 (1H, s), 7.85 (4H, m), 7.89 (2H, s), | | |
| | | | | 8.12 (1H, m) | | |
| TC10-109 | H109 | phenyl | phenyl | δ = 7.29 (1H, m), 7.33 (1H, m), 7.37 (4H, | 806.04 | 805.29 |
| | | | | m), 7.41 (2H, m), 7.46 (4H, m), | | |
| | | | | 7.48~7.52 (18H, m), 7.7 (2H, m), | | |
| | | | | 7.72 (1H, s), 7.75 (2H, m), 7.89 (2H, s), | | |
| | | | | 8.12 (1H, m), 8.63 (1H, s) | | |
| TC10-110 | H110 | phenyl | phenyl | δ = 7.05 (2H, m), 7.29~7.37 (6H, | 752.97 | 752.26 |
| | | | | 7.46~7.63 (14H, m), 7.72 (1H, s), | | |
| | | | | 7.89 (2H, s), 7.95~8 (6H, m), 8.12 | | |
| | | | | m), 8.38 (2H, m), 8.85 (2H, m) | | |

TABLE 3
TA49

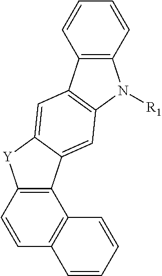

| | | | | | | MS/FAB | |
|---|---|---|---|---|---|---|---|
| Cmpd . | R₁ | Y | R₄ | R₅ | ¹H NMR (CDCl₃, 200 MHz) | found | calculated |
| TA49-1 | H1 | SiR₄R₅ | phenyl | phenyl | δ = 7.05 (2H, m), 7.25 (1H, m), | 702.91 | 702.25 |
| | | | | | 7.33~7.37 (5H, m), 7.46~7.47 (6H, | | |
| | | | | | m), 7.54~7.55 (8H, m), 7.66 (1H, m), | | |
| | | | | | 7.72 (1H, s), 7.89 (1H, s), 7.94 (1H, | | |
| | | | | | m), 8.08 (1H, m), 8.14 (1H, m), | | |
| | | | | | 8.3 (4H, m), 8.55 (2H, m) | | |
| TA49-4 | H4 | SiR₄R₅ | phenyl | phenyl | δ = 7.25 (1H, m), 7.33~7.55 (19H, m), | 703.90 | 703.24 |
| | | | | | 7.66 (1H, m), 7.72 (1H, s), 7.79 (4H, | | |
| | | | | | m), 7.89 (1H, s), 7.94 (1H, m), | | |
| | | | | | 8.08 (1H, m), 8.14 (1H, m), 8.55 (2H, | | |
| | | | | | m), 8.63 (1H, s) | | |
| TA49-5 | H5 | SiR₄R₅ | phenyl | phenyl | δ = 7.14 (2H, m), 7.25 (1H, m), | 704.89 | 704.24 |
| | | | | | 7.33~7.37 (5H, m), 7.46 (4H, m), | | |
| | | | | | 7.55 (4H, m), 7.66~7.7 (3H, m), | | |
| | | | | | 7.72 (1H, s), 7.89 (1H, s), 7.94 (1H, | | |
| | | | | | m), 8.08 (1H, m), 8.14~8.15 (3H, m), | | |
| | | | | | 8.53~8.55 (4H, m), 9.3 (2H, m) | | |
| TA49-12 | H12 | SiR₄R₅ | phenyl | phenyl | δ = 7.25 (1H, m), 7.33~7.55 (19H, m), | 704.89 | 704.24 |
| | | | | | 7.66 (1H, m), 7.72 (1H, s), 7.89 (1H, | | |
| | | | | | s), 7.94 (1H, m), 8.08 (1H, m), | | |
| | | | | | 8.14 (1H, m), 8.28 (4H, m), 8.55 (2H, | | |
| | | | | | m) | | |
| TA49-14 | H14 | SiR₄R₅ | phenyl | phenyl | δ = 7.25 (1H, m), 7.33 (1H, m), | 676.88 | 676.23 |
| | | | | | 7.37~7.46 (19H, m), 7.66 (1H, m), | | |
| | | | | | 7.72 (1H, s), 7.76 (1H, m), 7.89 (1H, | | |
| | | | | | s), 7.92~7.94 (2H, m), 8.08 (1H, m), | | |
| | | | | | 8.14 (1H, m), 8.43 (1H, s), 8.55 (2H, | | |
| | | | | | m) | | |
| TA49-42 | H42 | SiR₄R₅ | phenyl | phenyl | δ = 7.25~7.37 (9H, m), | 714.93 | 714.25 |
| | | | | | 7.46~7.55 (9H, m), 7.62~7.66 (6H, | | |
| | | | | | m), 7.72 (1H, s), 7.89 (1H, s), | | |
| | | | | | 7.94 (2H, m), 8.08~8.14 (3H, m), | | |
| | | | | | 8.55 (3H, m) | | |
| TA49-99 | H99 | SiR₄R₅ | phenyl | phenyl | δ = 7.29 (1H, m), 7.37 (10H, m), | 887.19 | 886.29 |
| | | | | | 7.46~7.55 (20H, m), 7.63~7.66 (2H, | | |
| | | | | | m), 7.72 (1H, s), 7.89 (3H, s), | | |
| | | | | | 8.08~8.14 (3H, m), 8.55 (1H, m), | | |
| | | | | | 8.59 (1H, s) | | |
| TA49-100 | H100 | SiR₄R₅ | phenyl | phenyl | δ = 7.29 (1H, m), 7.37 (10H, m), | 886.20 | 885.30 |
| | | | | | 7.46~7.55 (20H, m), 1.63~7.66 (2H, | | |
| | | | | | m), 7.72 (1H, s), 7.89 (3H, s), | | |
| | | | | | 7.96 (1H, m), 8.08~8.14 (3H, m), | | |
| | | | | | 8.55~8.57 (2H, m) | | |
| TA49-101 | H101 | SiR₄R₅ | phenyl | phenyl | δ = 7.29~7.31 (2H, m), 7.37 (10H, m), | 885.21 | 884.30 |
| | | | | | 7.45~7.55 (21H, m), 7.63~7.66 (2H, | | |
| | | | | | m), 7.72 (1H, s), 7.89 (1H, s), | | |
| | | | | | 8.08~8.14 (3H, m), 8.38~8.4 (3H, | | |
| | | | | | m), 8.55 (1H, m) | | |
| TA49-102 | H102 | SiR₄R₅ | phenyl | phenyl | δ = 7.29 (1H, m), 7.37~7.55 (29H, | 887.18 | 886.29 |
| | | | | | 7.61~7.66 (3H, m), 7.72 (1H, s) | | |
| | | | | | 7.76 (1H, m), 7.89 (1H, s), | | |
| | | | | | 8.08~8.14 (3H, m), 8.38 (1H, m) | | |
| | | | | | 8.55 (1H, m), 8.59 (1H, s) | | |
| TA49-103 | H103 | SiR₄R₅ | phenyl | phenyl | δ = 7.29 (1H, m), 7.37~7.55 (29H, m), | 886.20 | 885.30 |
| | | | | | 7.61~7.66 (3H, m), 7.72 (1H, s), | | |
| | | | | | 7.76 (1H, m), 7.89 (2H, s), 7.96 (1H, | | |
| | | | | | m), 8.08~8.14 (3H, m), | | |
| | | | | | 8.55~8.57 (2H, m) | | |
| TA49-104 | H104 | SiR₄R₅ | phenyl | phenyl | δ = 7.29~7.31 (2H, m), 7.37 (10H, m), | 885.21 | 884.30 |
| | | | | | 7.45~7.55 (20H, m), 7.63~7.66 (3H, | | |
| | | | | | m), 7.72 (1H, s), 7.89 (1H, s), | | |
| | | | | | 8.08~8.14 (3H, m), 8.27 (1H, m), | | |
| | | | | | 8.38~8.4 (2H, m), 8.55 (1H, m) | | |
| TA49-105 | H105 | SiR₄R₅ | phenyl | phenyl | S = 7.29 (1H, m), 7.37~7.55 (16H, | 704.89 | 704.24 |
| | | | | | 7.63~7.68 (4H, m), 7.72 (1H, s), | | |
| | | | | | 7.79 (2H, m), 7.89 (1H, s), | | |
| | | | | | 8.08~8.14 (3H, m), 8.28 (2H, m), | | |
| | | | | | 8.31 (1H, s), 8.55 (1H, m) | | |
| TA49-106 | H106 | SiR₄R₅ | phenyl | phenyl | δ = 7.29 (1H, m), 7.37~7.56 (17H, m), | 703.90 | 703.24 |
| | | | | | 7.63~7.68 (4H, m), 7.72 (1H, s), | | |
| | | | | | 7.79 (4H, m), 7.89 (1H, s), | | |
| | | | | | 8.08~8.14 (3H, m), 8.54~8.55 (2H, | | |
| | | | | | m) | | |
| TA49-107 | H107 | SiR₄R₅ | phenyl | phenyl | δ = 7.29 (1H, m), 7.37 (4H, m), | 702.91 | 702.25 |
| | | | | | 7.46~7.55 (12H, m), 7.63~7.68 (4H, | | |
| | | | | | m), 7.72 (1H, s), 7.79 (2H, m), | | |
| | | | | | 7.89 (1H, s), 8 (1H, m), | | |
| | | | | | 8.08~8.14 (3H, m), 8.3 (2H, m), | | |
| | | | | | 8.44 (1H, m), 8.55~8.6 (2H, m) | | |
| TA49-108 | H108 | SiR₄R₅ | phenyl | phenyl | δ = 7.25~7.29 (5H, m), | 857.08 | 856.30 |
| | | | | | 7.37~7.55 (23H, m), 7.63~7.66 (2H, | | |
| | | | | | m), 7.72 (1H, s), 7.85 (4H, m), | | |
| | | | | | 7.89 (1H, s), 8.08~8.14 (3H, m), | | |
| | | | | | 8.55 (1H, m) | | |

TABLE 4
TB52

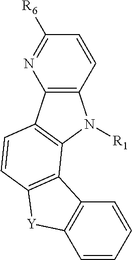

| | | | | | | | MS/FAB | |
|---|---|---|---|---|---|---|---|---|
| Cmpd. | R₁ | Y | R₂ | R₃ | R₆ | ¹H NMR(CDCl₃, 200 MHz) | found | calculated |
| TB52-1 | H1 | CR₂R₃ | methyl | methyl | phenyl | δ = 7.05 (2H, m), 7.25 (1H, m), | 702.91 | 702.25 |
| | | | | | | 7.33~7.37 (5H, m), 7.46~7.47 (6H, | | |
| | | | | | | m) , 7.54~7.55 (8H, m), 7.66 (1H, m), | | |
| | | | | | | 7.72 (1H, s), 7.89 (1H, s), 7.94 (1H, | | |
| | | | | | | m), 8.08 (1H, m), 8.14 (1H, m), | | |
| | | | | | | 8.3 (4H, m), 8.55 (2H, m) | | |
| TB52-99 | H99 | CR₂R₃ | methyl | methyl | H | δ = 7.25 (1H, m), 7.33~7.55 (19H, m), | 703.90 | 703.24 |
| | | | | | | 7.66 (1H, m), 7.72 (1H, s), 7.79 (4H, | | |
| | | | | | | m), 7.89 (1H, s), 7.94 (1H, m), | | |
| | | | | | | 8.08 (1H, m), 8.14 (1H, m), 8.55 (2H, | | |
| | | | | | | m), 8.63 (1H, s) | | |
| TB52-100 | H100 | CR₂R₃ | methyl | methyl | H | δ = 7.14 (2H, m), 7.25 (1H, m), | 704.89 | 704.24 |
| | | | | | | 7.33~7.37 (5H, m), 7.46 (4H, m), | | |
| | | | | | | 7.55 (4H, m), 7.66~7.7 (3H, m), | | |
| | | | | | | 7.72 (1H, s), 7.89 (1H, s), 7.94 (1H, | | |
| | | | | | | m), 8.08 (1H, m), 8.14~8.15 (3H, m), | | |
| | | | | | | 8.53~8.55 (4H, m), 9.3 (2H, m) | | |
| TB52-104 | H104 | CR₂R₃ | methyl | methyl | H | δ = 7.25 (1H, m), 7.33~7.55 (19H, m), | 704.89 | 704.24 |
| | | | | | | 7.66 (1H, m), 7.72 (1H, s), 7.89 (1H, | | |
| | | | | | | s), 7.94 (1H, m), 8.08 (1H, m), | | |
| | | | | | | 8.14 (1H, m), 8.28 (4H, m), 8.55 (2H, | | |
| | | | | | | m) | | |
| TB52-106 | H106 | CR₂R₃ | phenyl | phenyl | H | δ = 7.25 (1H, m), 7.33 (1H, m), | 676.88 | 676.23 |
| | | | | | | 7.37~7.46 (19H, m), 7.66 (1H, m), | | |
| | | | | | | 7.72 (1H, s), 7.76 (1H, m), 7.89 (1H, | | |
| | | | | | | s), 7.92~7.94 (2H, m), 8.08 (1H, m), | | |
| | | | | | | 8.14 (1H, m), 8.43 (1H, s), 8.55 (2H, | | |
| | | | | | | m) | | |

### Example 1

### Manufacture of OLED Device Using the Organic Electroluminescent Compound According to the Present Invention

An OLED device was manufactured using the electroluminescent material according to the present invention. First, a transparent electrode ITO thin film (15 Ω/□) obtained from a glass for OLED (produced by Samsung Corning) was subjected to ultrasonic washing with trichloroethylene, acetone, ethanol and distilled water, sequentially, and stored in isopropanol before use. Then, an ITO substrate was equipped in a substrate folder of a vacuum vapor deposition apparatus, and 4,4′,4″-tris(N,N-(2-naphthyl)-phenylamino)triphenylamine (2-TNATA) was placed in a cell of the vacuum vapor deposition apparatus, which was then ventilated up to 10⁻⁶ torr of vacuum in the chamber.

Then, electric current was applied to the cell to evaporate 2-TNATA, thereby forming a hole injection layer having a thickness of 60 nm on the ITO substrate. Then, N,N′-bis(α-naphthyl)-N,N′-diphenyl-4,4′-diamine (NPB) was placed in another cell of the vacuum vapor deposition device, and electric current was applied to the cell to evaporate NPB, thereby forming a hole transport layer having a thickness of 20 nm on the hole injection layer.

An electroluminescent layer was formed on the hole transport layer as follows. The compound according to the present invention (e.g., Compound TC10-19) vacuum sublimed at 10⁻⁶ torr was filled in a cell of a vacuum vapor deposition apparatus as a host material, and an electroluminescent dopant (e.g., (piq)₂Ir(acac)[bis-(1-phenylisoquinolyl)iridium(III)acetylacet onate]) was filled in another cell. The two materials were evaporated at different speed, so that an electroluminescent layer having a thickness of 30 nm was formed on the hole transport layer at 4 to 10 mol %.

Then, tris(8-hydroxyquinoline)-aluminum(III) (Alq) was vapor-deposited as an electron transport layer with a thickness of 20 nm, and lithium quinolate (Liq) was vapor-deposited as an electron injecting layer with a thickness of 1 to 2 nm. Thereafter, an Al cathode was vapor-deposited with a thickness of 150 nm by using another vacuum vapor-deposit device to manufacture an OLED.

### Example 2

### Manufacture of an OLED by Using the Organic Electroluminescent Compound According to the Present Invention

An OLED was manufactured in the same manner as Example 1, except that a compound according to the present invention (e.g.: Compound TC10-12) as host material was used and bis(2-methyl-8-quinolinato) (p-phenylphenolato)aluminum(III) (BAlq) having a thickness of 5 nm was vapor deposited as a hole blocking layer on the electroluminescent layer.

### Example 3

### Manufacture of an OLED by Using the Organic Electroluminescent Compound According to the Present Invention

An OLED was manufactured in the same manner as Example 1, except that a compound according to the present invention (e.g.: Compound TC10-97) as host material and an organic iridium complex (Ir(ppy)₃[tris(2-phenylpyridine)iridium]) as electroluminescent dopant were used.

### Example 4

### Manufacture of an OLED by Using the Organic Electroluminescent Compound According to the Present Invention

An OLED was manufactured in the same manner as Example 3, except that a compound according to the present invention (e.g.: Compound TC4-105) as host material and bis(2-methyl-8-quinolinato) (p-phenylphenolato)aluminum(III) (BAlq) having a thickness of 5 nm was vapor deposited as a hole blocking layer on the electroluminescent layer.

### Comparative Example 1 and 2

### Manufacture of an OLED Using Conventional Electroluminescent Material

An OLED was manufactured in the same manner as Examples 2 and 4 except that 4,4′-di(9H-carbazol-9-one)biphenyl(CBP) instead of the organic electroluminescent compound according to the present invention was used as electroluminescent host material in another cell of the vacuum vapor deposition device.

The luminous efficiencies of the OLED's comprising the organic electroluminescent compound according to the present invention (Examples 1 to 4 and Comparative Examples 1 and 2) or conventional EL compounds were measured at 1,000 cd/m², respectively, and the results are shown in Table 5.

TABLE 5

| | | | | | Driving | Power efficiency | |
|---|---|---|---|---|---|---|---|
| | | | | Hole blocking | voltage(V) | (lm/W) | |
| No. | | Host | Dopant | layer | @1,000 cd/m² | @1,000 cd/m² | Color |
| Example | 1 | TC10-19 | (piq)₂Ir(acac) | — | 6.6 | 3.2 | red |
| 1 | 2 | TC10-108 | (piq)₂Ir(acac) | — | 6.4 | 3.4 | red |
| | 3 | TC4-102 | (piq)₂Ir(acac) | — | 6.6 | 3.4 | red |
| | 4 | TC10-60 | (piq)₂Ir(acac) | — | 6.7 | 3.1 | red |
| | 5 | TC4-12 | (piq)₂Ir(acac) | — | 6.4 | 3.8 | red |
| | 6 | TC4-59 | (piq)₂Ir(acac) | — | 6.3 | 4.2 | red |
| Example | 7 | TC10-12 | (piq)₂Ir(acac) | BAlq | 6.9 | 3.3 | red |
| 2 | 8 | TC10-83 | (piq)₂Ir(acac) | BAlq | 7.0 | 3.0 | red |
| | 9 | TC10-100 | (piq)₂Ir(acac) | BAlq | 6.8 | 3.4 | red |
| | 10 | TA49-103 | (piq)₂Ir(acac) | BAlq | 6.9 | 3.3 | red |
| Example | 11 | TC10-97 | Ir(ppy)₃ | — | 6.2 | 13.5 | green |
| 3 | 12 | TC10-52 | Ir(ppy)₃ | — | 6.1 | 14.8 | green |
| | 13 | TC4-103 | Ir(ppy)₃ | — | 6.4 | 14.0 | green |
| | 14 | TC4-106 | Ir(ppy)₃ | — | 6.1 | 14.1 | green |
| | 15 | TC10-4 | Ir(ppy)₃ | — | 6.3 | 13.4 | green |
| | 16 | TC4-82 | Ir(ppy)₃ | — | 6.5 | 13.7 | green |
| Example | 17 | TC4-105 | Ir(ppy)₃ | BAlq | 6.6 | 13.1 | green |
| 4 | 18 | TC4-96 | Ir(ppy)₃ | BAlq | 6.8 | 13.0 | green |
| | 19 | TC10-106 | Ir(ppy)₃ | BAlq | 6.8 | 12.7 | green |
| | 20 | TC4-50 | Ir(ppy)₃ | BAlq | 6.9 | 13.2 | green |

| | | | | | | |
|---|---|---|---|---|---|---|
| Comparative | CBP | (piq)₂Ir(acac) | BAlq | 7.5 | 2.6 | red |
| Example 1 | | | | | | |
| Comparative | CBP | Ir(ppy)₃ | BAlq | 7.5 | 10.5 | green |
| Example 2 | | | | | | |

As shown in Table 5, the organic electroluminescent compounds according to the present invention have excellent properties compared with the conventional material. In addition, the device using the organic electroluminescent compound according to the present invention as host material for emitting red or green color has excellent electroluminescent properties and drops driving voltage, thereby increasing power efficiency and improving power consumption.

What is claimed is:

1. An organic electroluminescent compound is represented by Chemical Formulas 1 to 6: 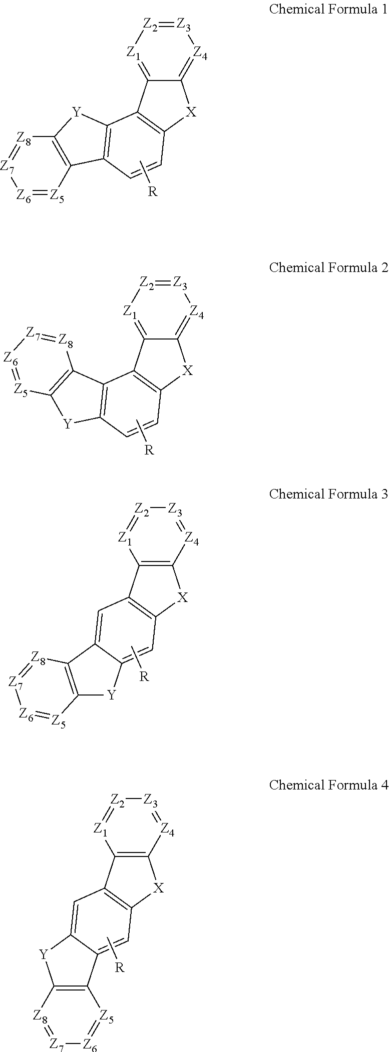 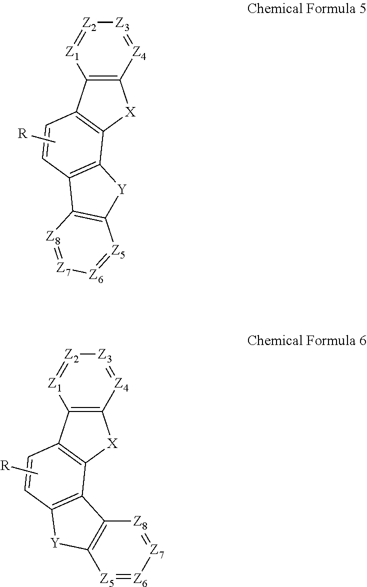
wherein,
X and Y independently represent N(R₁), C(R₂) (R₃) or Si (R₄)(R₅), with proviso that at least one of X and Y is (are) N(R₁) and the remaining is C(R₂)(R₃) or Si(R₄)(R₅);
Z₁ through Z₈ independently represent C(R₆) or N, wherein R₆ may be different from each other, and the neighboring R₆ may be linked to each other to form a ring;
R₁ through R₅ independently represent (C1-C30)alkyl, (C3-C30)cycloalkyl, 5- to 7-membered heterocycloalkyl, (C2-C30)alkenyl, (C2-C30)alkynyl, (C6-C30)aryl or (C3-C30)heteroaryl;
R and R₆ independently represent hydrogen, (C1-C30)alkyl, halogen, cyano, (C3-C30)cycloalkyl, 5- to 7-membered heterocycloalkyl, (C2-C30)alkenyl, (C2-C30)alkynyl, (C6-C30)aryl, (C3-C30)heteroaryl, mono- or di(C1-C30)alkylamino, mono- or di(C6-C30)arylamino, R^{a}R^{b}R^{c}Si— [wherein R^{a}, R^{b} and R^{c} independently represent (C1-C30) alkyl or (C6-C30) aryl], R^{d}Y— [wherein Y represents O or S, and R^{d} represents (C1-C30)alkyl or (C6-C30)aryl], mono- or di(C6-C30)arylboranyl, mono- or di(C1-C60)alkylboranyl, nitro or hydroxyl;
the alkyl, cycloalkyl, heterocycloalkyl, alkenyl, alkynyl, aryl, heteroaryl, alkylamino, arylamino, arylboranyl or alkylboranyl of R and R₁ through R₆ and the alkyl or aryl of R^{a}, R^{b}, R^{c} and R^{d} may be further substituted by one or more substituent(s) selected from a group consisting of (C1-C30)alkyl, halogen, cyano, (C3-C30)cycloalkyl, 5- to 7-membered heterocycloalkyl, (C2-C30)alkenyl, (C2-C30)alkynyl, (C6-C30)aryl, (C1-C30)alkoxy, (C6-C30)aryloxy, (C6-C30)aryl substituted by P(═O)R^{e}R^{f} [wherein R^{e} and R^{f} independently represent (C1-C30)alkyl or (C6-C30)aryl], (C3-C30)heteroaryl, (C3-C30)heteroaryl substituted by (C6-C30)aryl, (C3-C30)heteroaryl substituted by (C1-C30)alkyl, (C6-C30)ar(C1-C30)alkyl, (C6-C30)arylthio, (C1-C30)alkylthio, mono- or di(C1-C30)alkylamino, mono- or di(C6-C30)arylamino, tri(C1-C30)alkylsilyl, di(C1-C30)alkyl(C6-C30)arylsilyl, tri(C6-C30)arylsilyl, mono- or di(C6-C30)arylboranyl, mono- or di(C1-C30)alkylboranyl, nitro and hydroxyl; and
the heterocycloalkyl or heteroaryl may contain one or more heteroatom(s) selected from B, N, O, S, P(═O), Si and P.

2. An organic electroluminescent compound according to claim 1, which is selected from the following compounds: 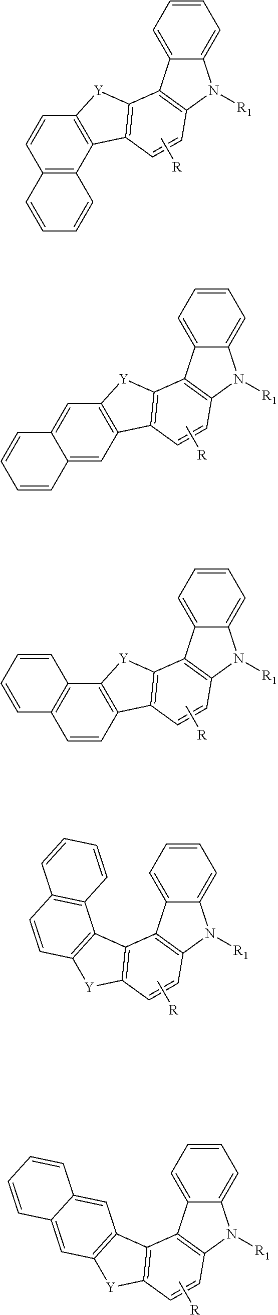 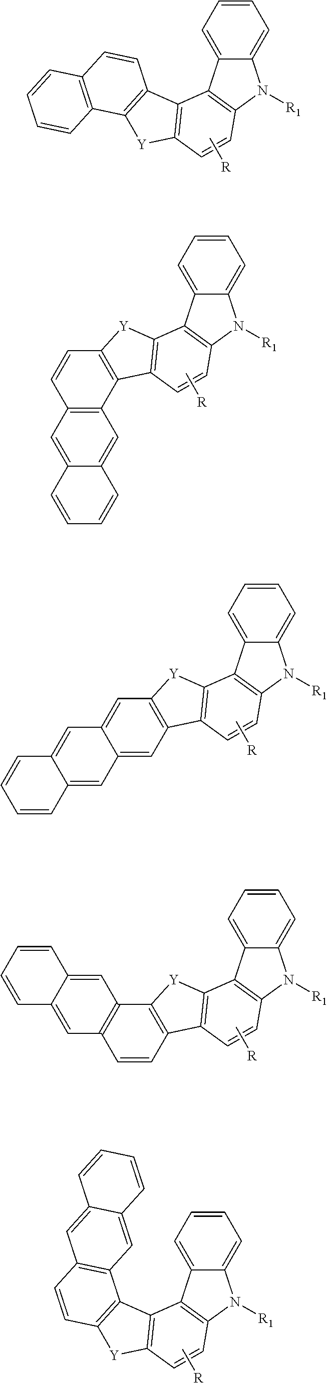 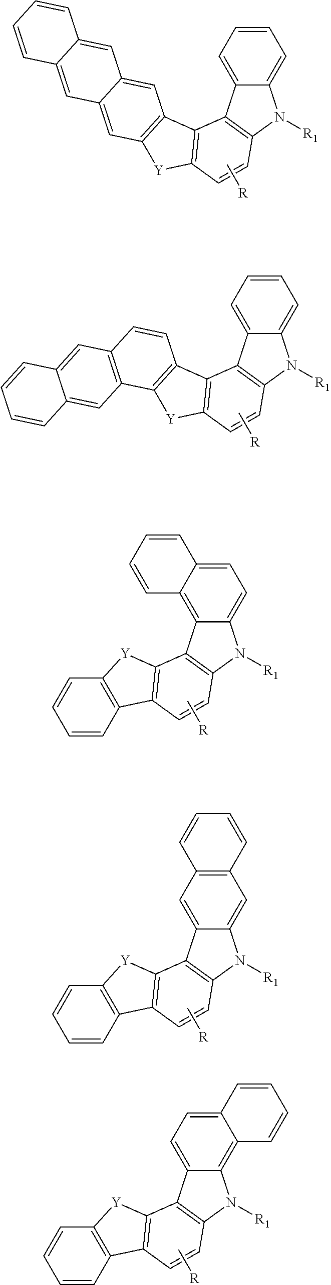 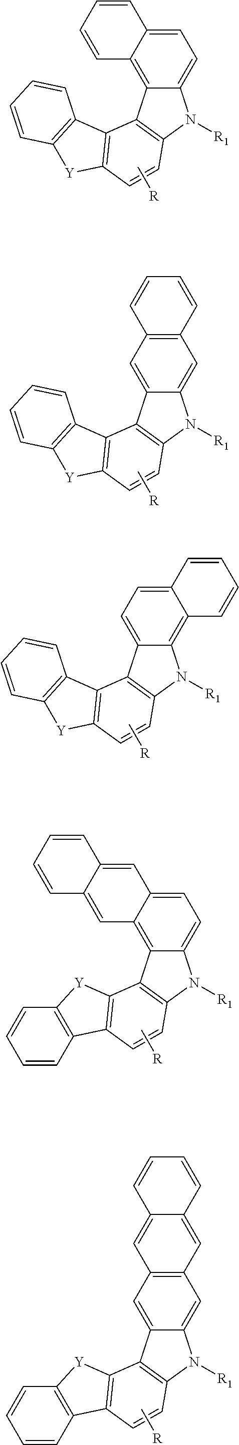 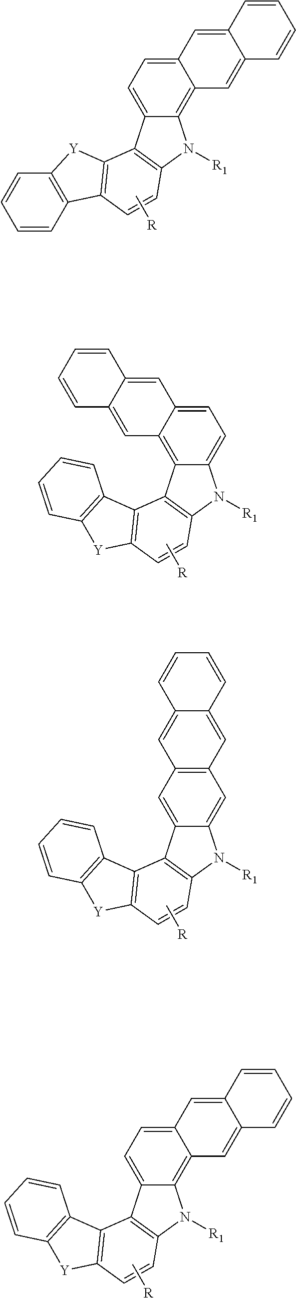 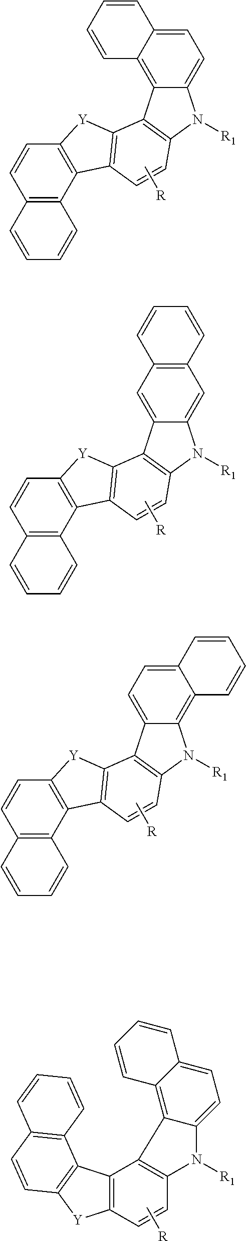 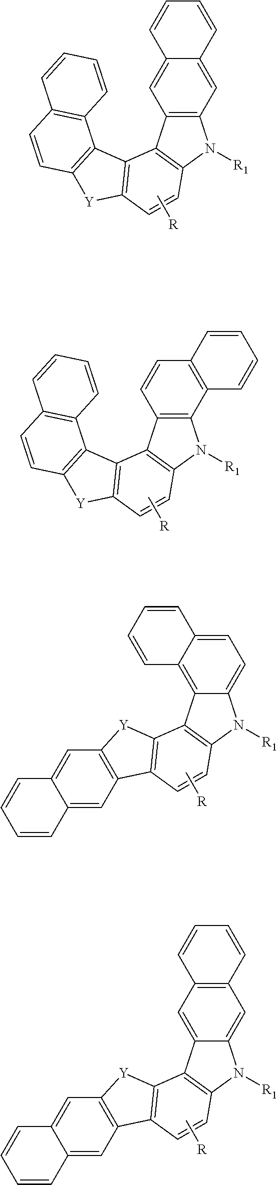 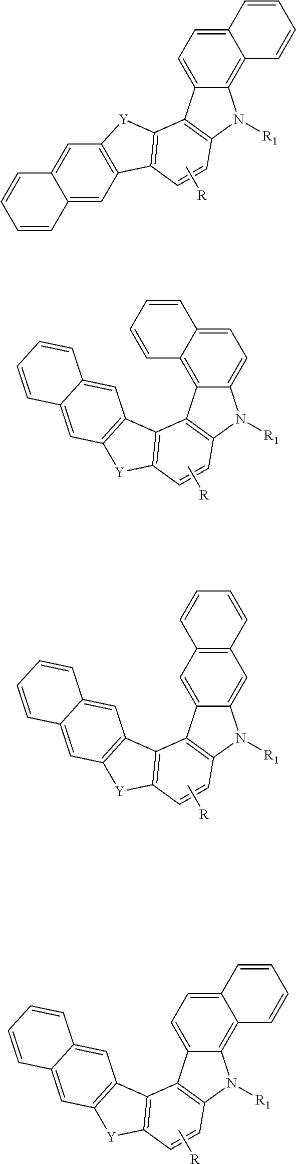 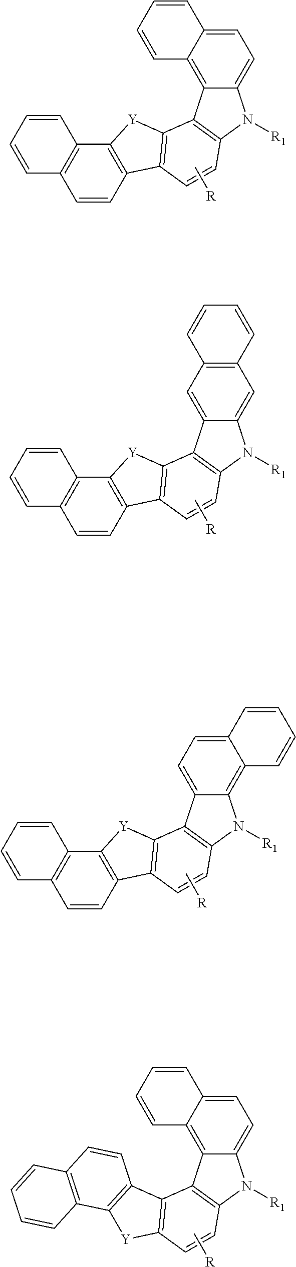 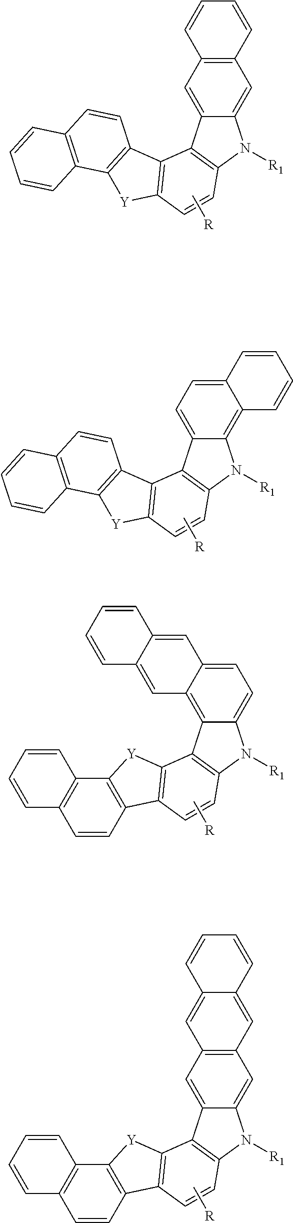 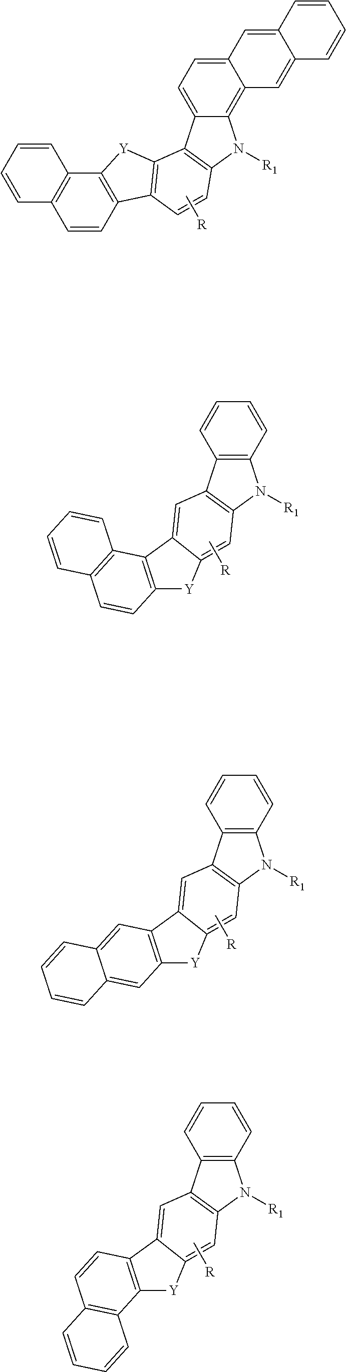 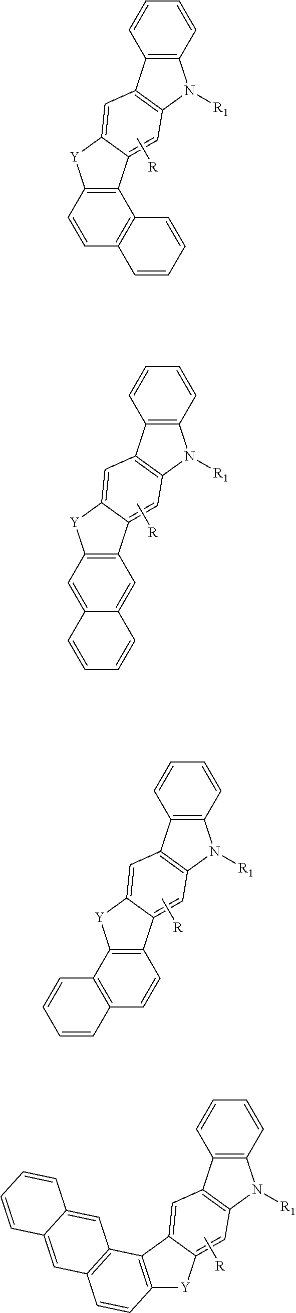 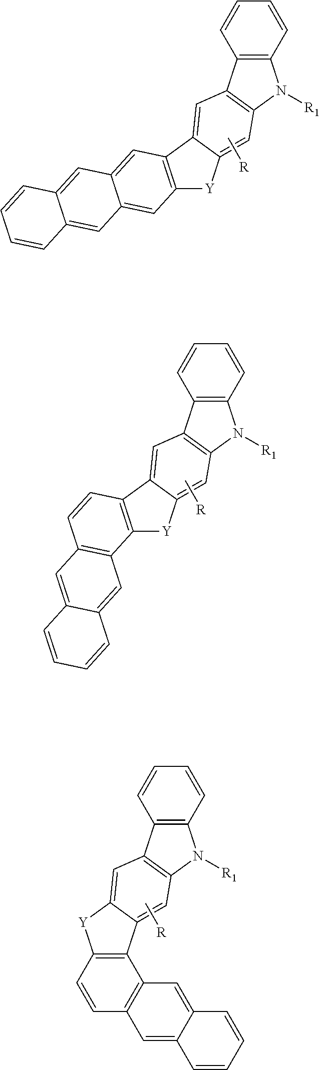 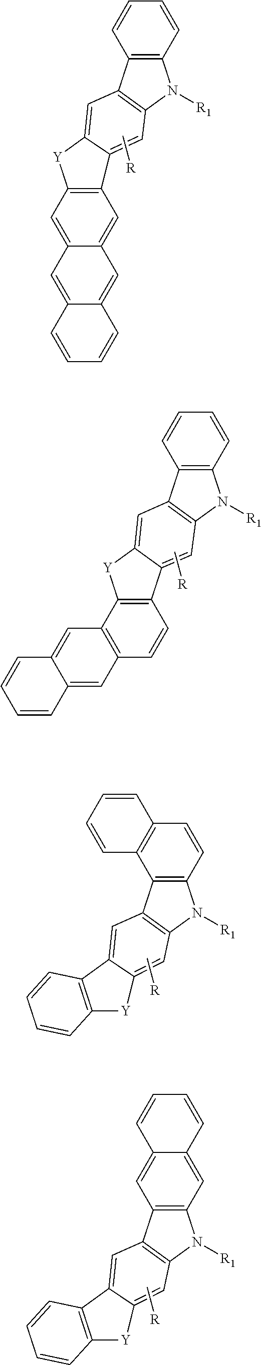 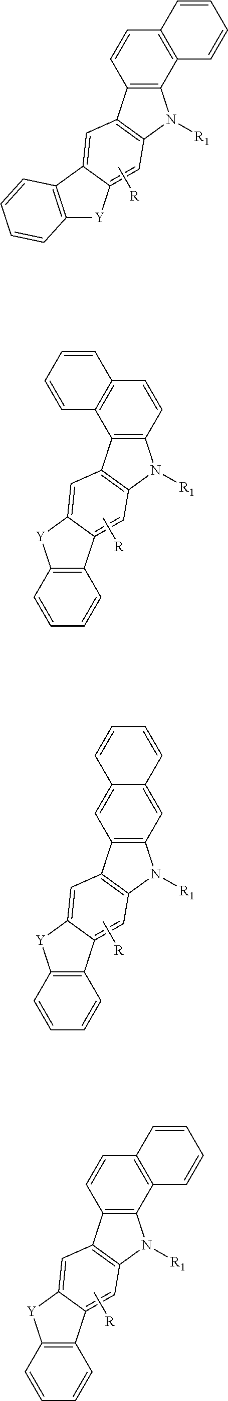 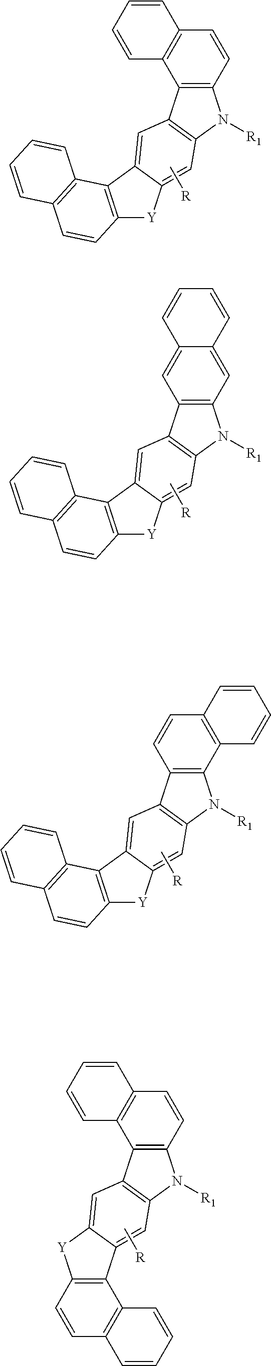 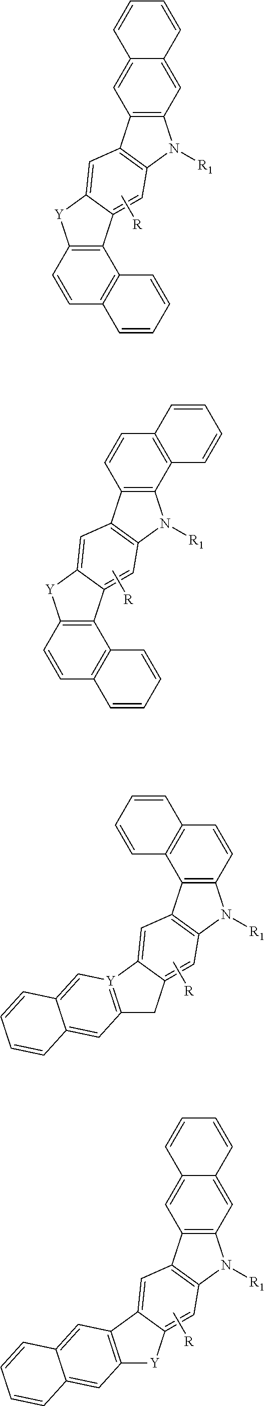 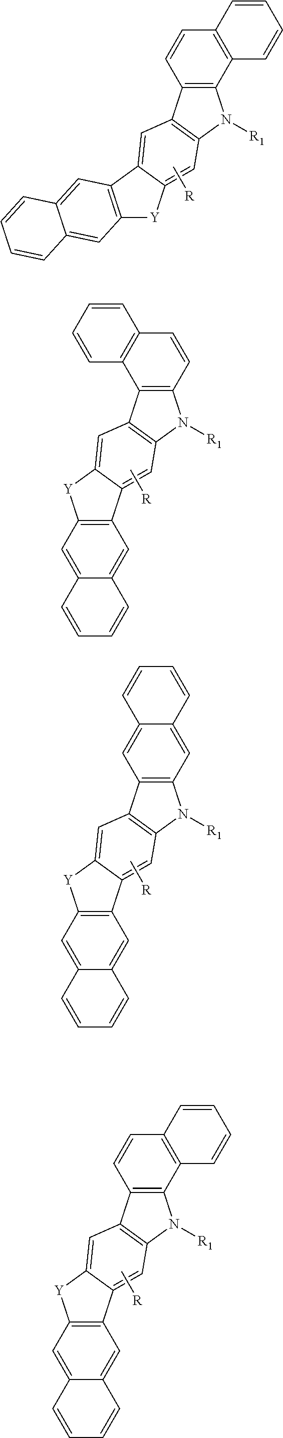 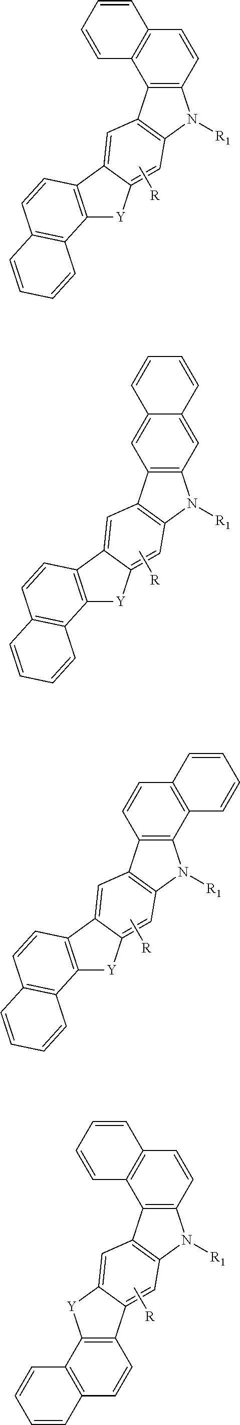 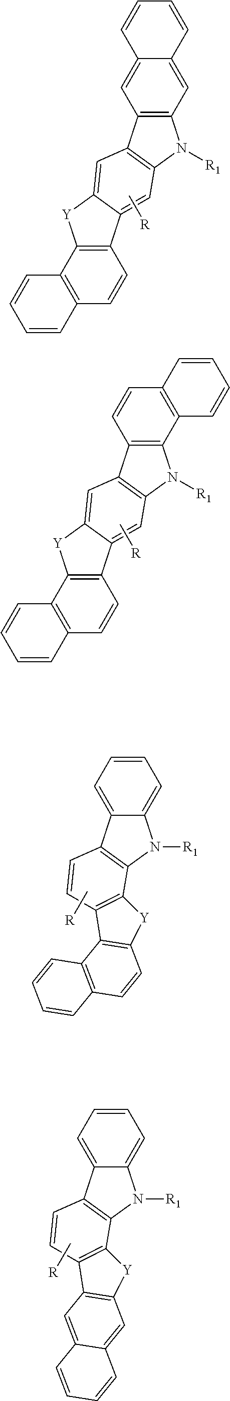 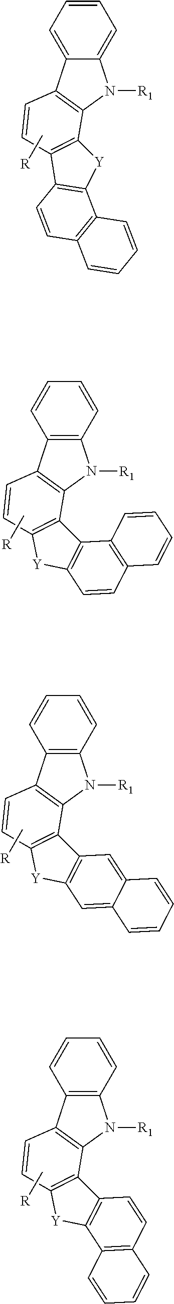 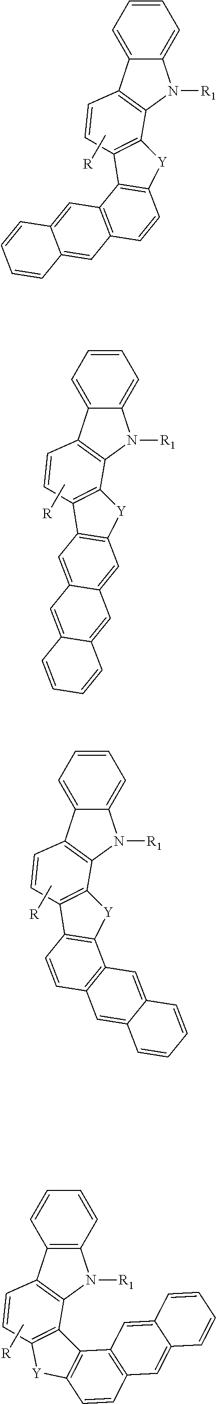 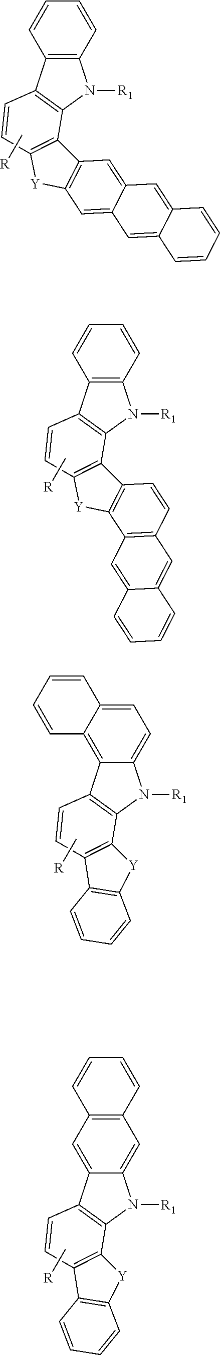 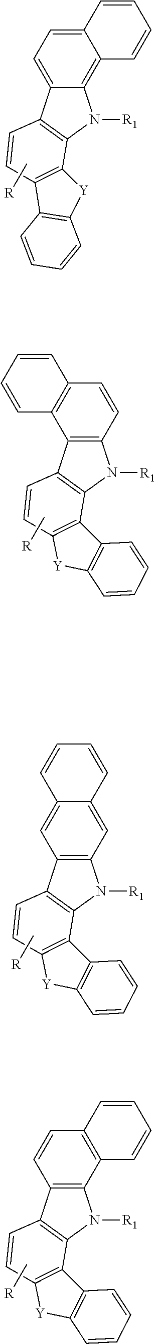 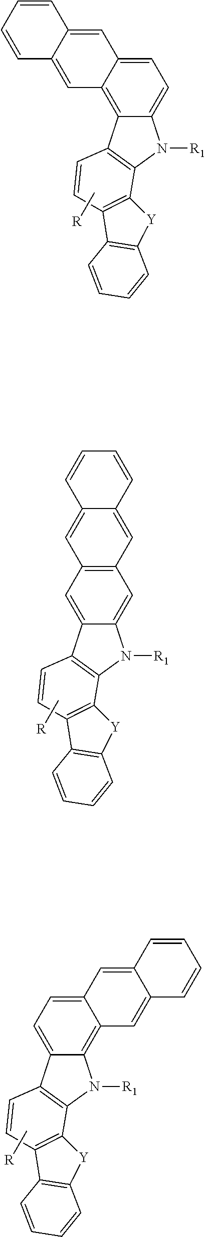 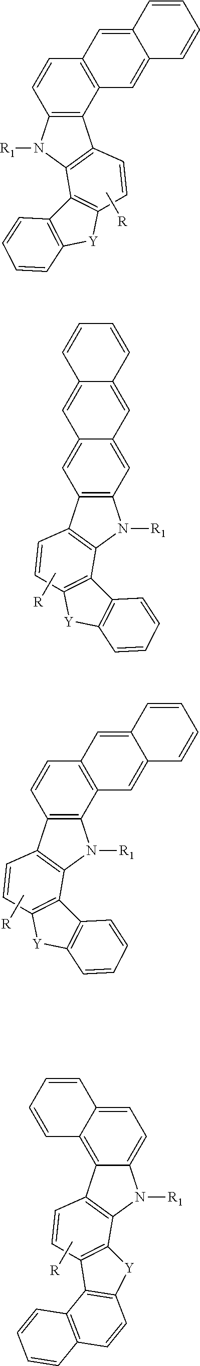 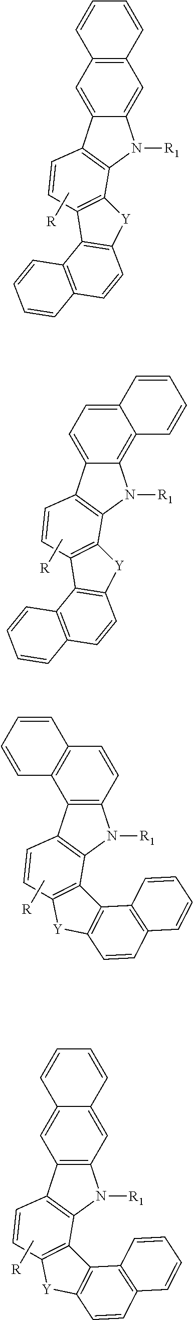 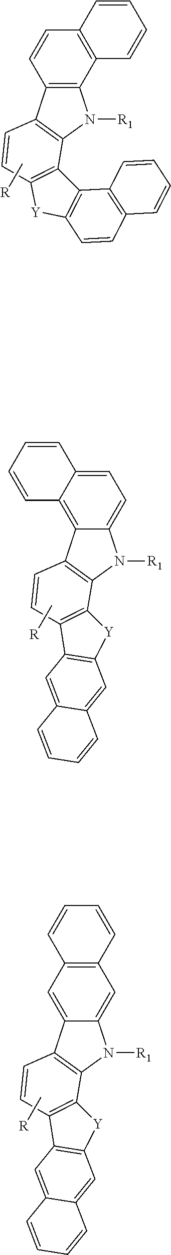 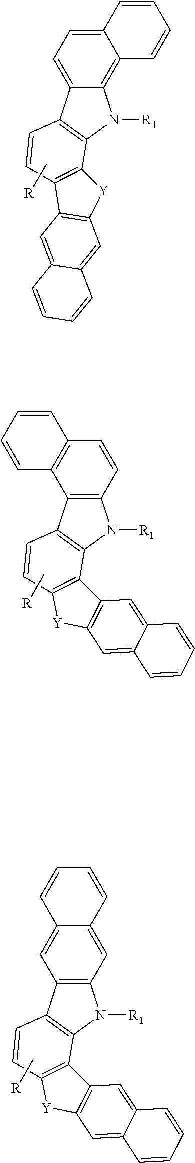 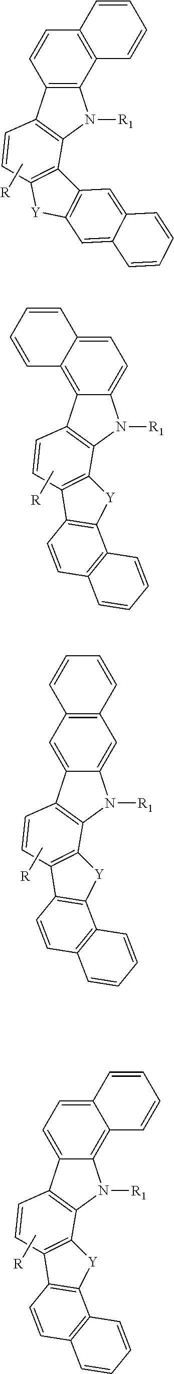 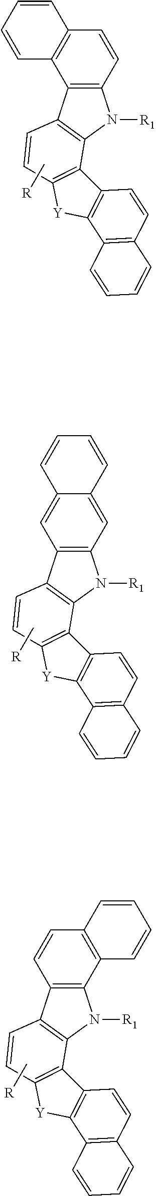
wherein,
Y represents C(R₂)(R₃) or Si(R₄)(R₅); and R and R₁ through R₅ are the same as defined in claim 1.

3. An organic electroluminescent compound according to claim 1, which is selected from the following compounds: 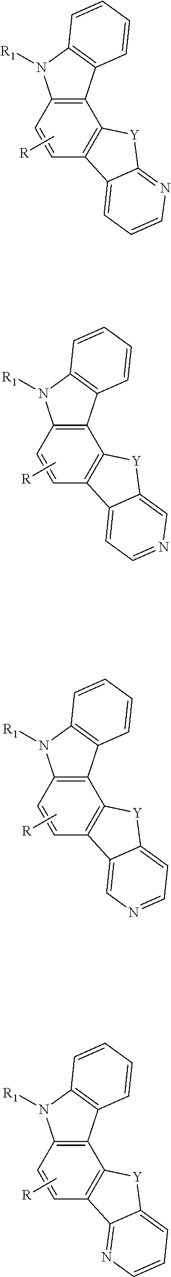 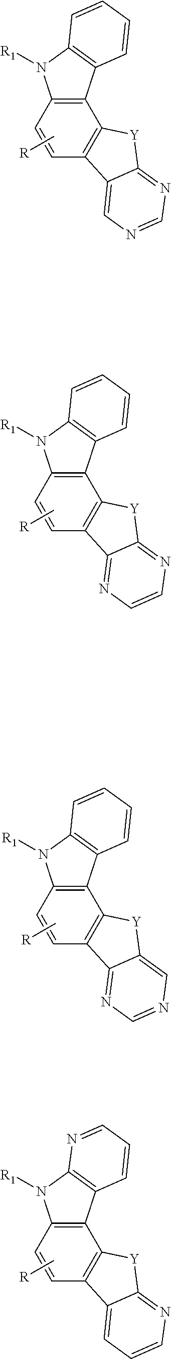 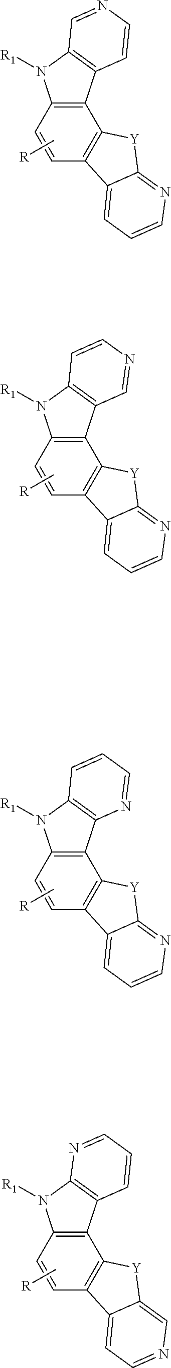 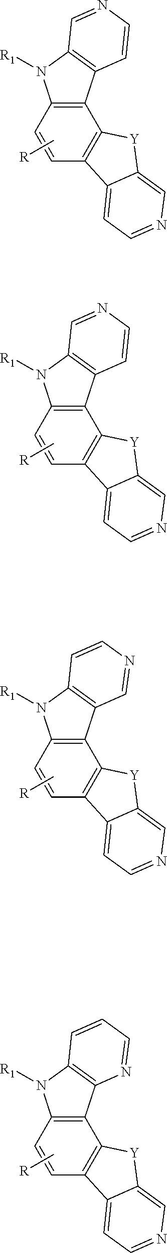 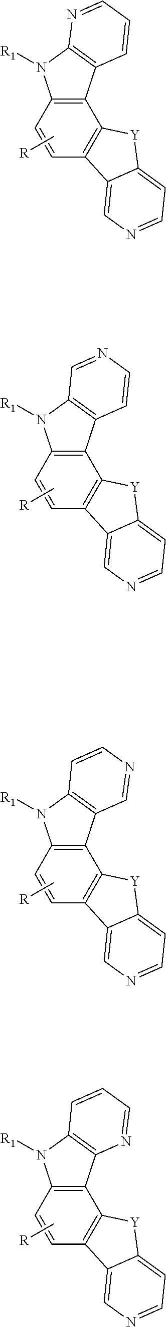 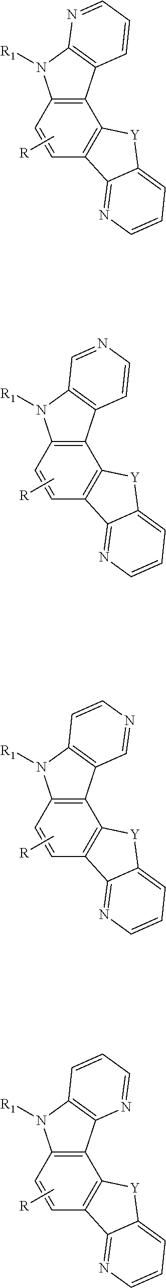 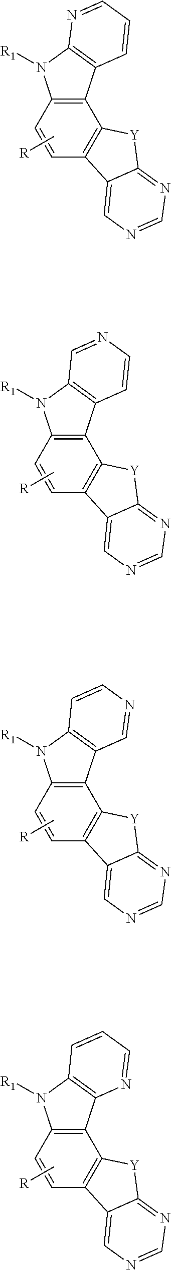 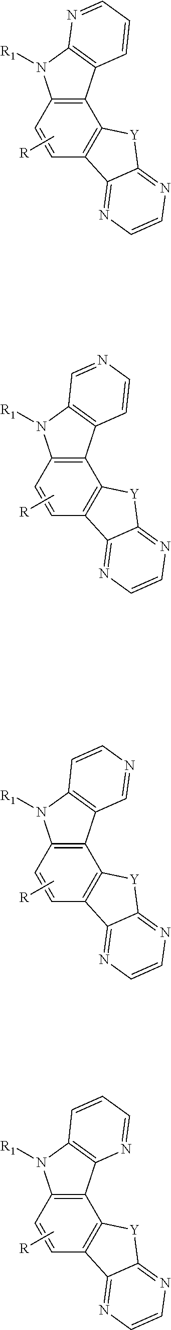 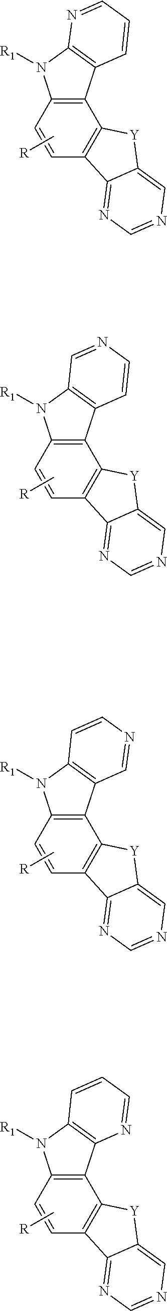 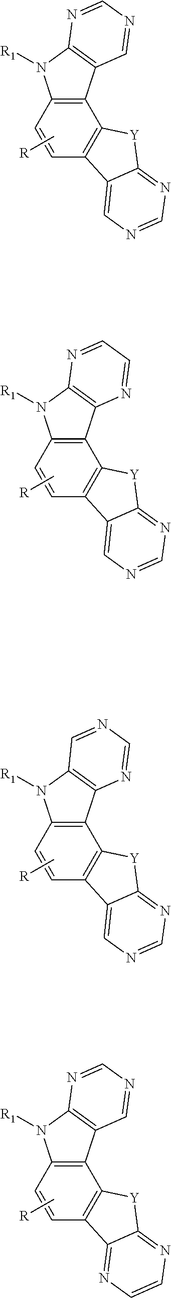 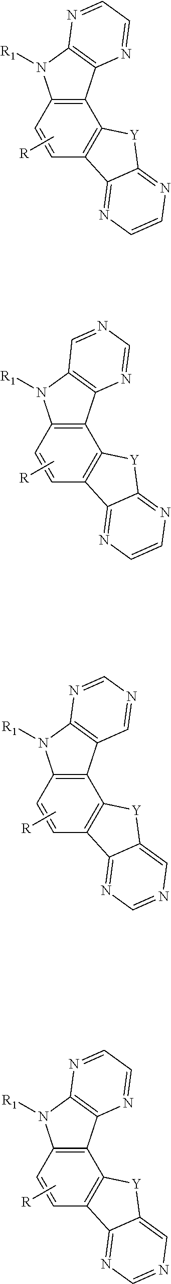 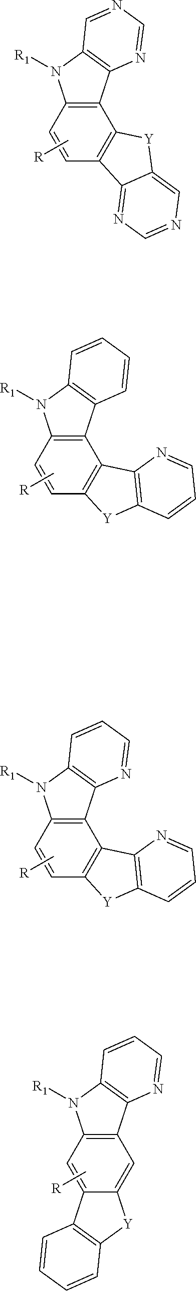 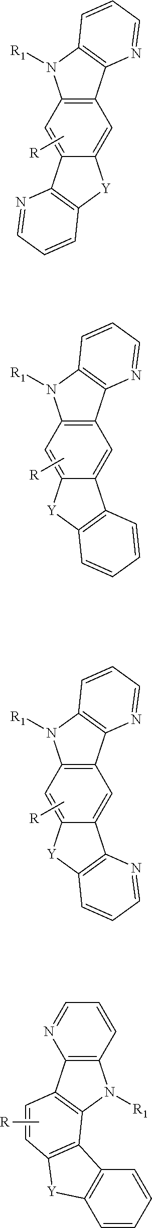
wherein,
Y represents C(R₂)(R₃) or Si(R₄)(R₅); and R and R₁ through R₅ are the same as defined in claim 1.

4. An organic electroluminescent compound according to claim 1, which is selected from the following compounds: 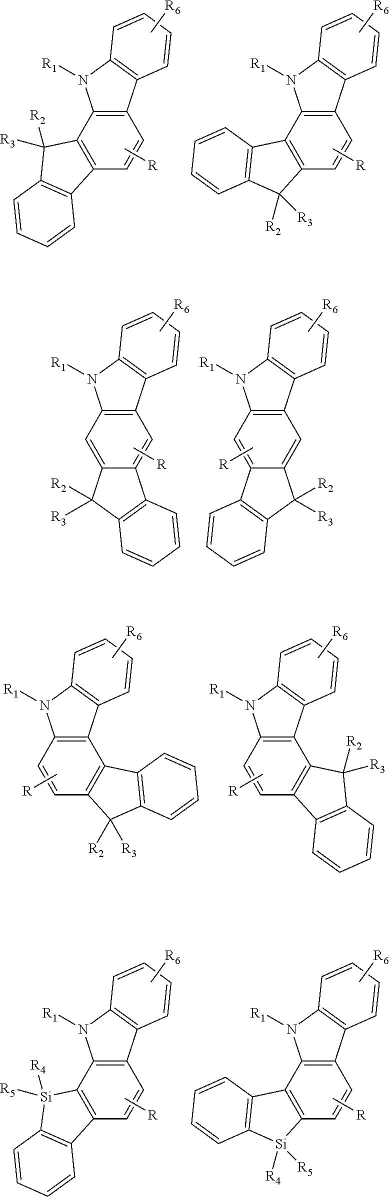 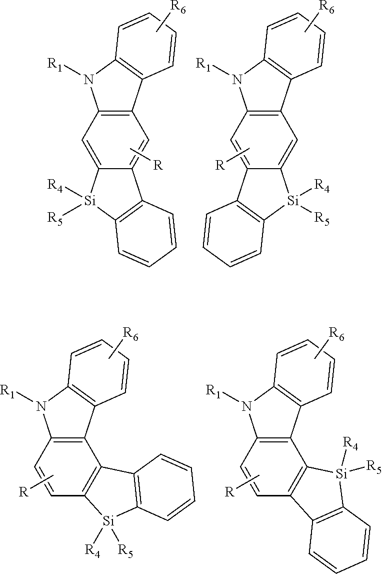
wherein,
R and R₁ through R₆ are the same as defined in claim 1.

5. An organic electroluminescent device comprising the organic electroluminescent compound according to any of claims 1 to 4.

6. The organic electroluminescent device according to claim 5, which comprises a first electrode; a second electrode; and one or more organic layer(s) interposed between the first electrode and the second electrode, wherein the organic layer comprises one or more organic electroluminescent compound(s) according to any of claims 1 to 4 and one or more phosphorescent dopant(s).

7. The organic electroluminescent device according to claim 6, wherein the organic layer further comprises one or more amine compound(s) selected from a group consisting of arylamine compounds and styrylarylamine compounds.

8. The organic electroluminescent device according to claim 6, wherein the organic layer further comprises one or more metal (s) selected from a group consisting of organic metals of Group 1, Group 2, 4th period and 5th period transition metals, lanthanide metals and d-transition elements or complex compound(s).

9. The organic electroluminescent device according to claim 6, wherein the organic layer comprises an electroluminescent layer and a charge generating layer.

10. The organic electroluminescent device according to claim 6, which is a white light-emitting organic electroluminescent device wherein the organic layer further comprises one or more organic electroluminescent layer(s) emitting blue, red or green light.
